(19) United States
(12) Patent Application Publication
(10) Pub. No.: US 2022/0016101 A1
(43) Pub. Date: Jan. 20, 2022

(54) METHODS OF TREATING DEPRESSION, ANXIETY AND SEXUAL DYSFUNCTION USING THE COMPOUND PIMAVANSERIN

(71) Applicant: ACADIA Pharmaceuticals Inc., San Diego, CA (US)

(72) Inventors: Ethan S. Burstein, San Diego, CA (US); Srdjan R. Stankovic, Flemington, NJ (US); Bryan Dirks, Princeton, NJ (US); Maurizio Fava, Boston, MA (US); James Randall Owen, Princeton, NJ (US); Daryl DeKarske, San Diego, CA (US)

(73) Assignee: ACADIA Pharmaceuticals Inc., San Diego, CA (US)

(21) Appl. No.: 17/290,479

(22) PCT Filed: Oct. 30, 2019
(86) PCT No.: PCT/US2019/058927

§ 371 Date: Apr. 30, 2021

Related U.S. Application Data
(60) Provisional application No. 62/905,708, filed on Sep. 25, 2019.
(60) Provisional application No. 62/856,312, filed on Jun. 3, 2019.
(60) Provisional application No. 62/837,915, filed on Apr. 24, 2019.
(60) Provisional application No. 62/812,366, filed on Mar. 1, 2019.
(60) Provisional application No. 62/752,990, filed on Oct. 30, 2018.

(51) Int. Cl. A61K 31/4468; A61K 9/00; A61P 15/10; A61P 15/12; A61P 25/24; A61P 25/16; A61P 25/22; A61K 45/06

(52) U.S. Cl. CPC A61K 31/4468; A61K 9/0053; A61P 15/10; A61K 45/06; A61P 25/24; A61P 25/16; A61P 25/22; A61P 15/12

(57) ABSTRACT

The disclosure provides, in part, a method of treating anxious distress with major depressive disorder in a patient in need thereof, comprising administering to the patient N-(4-fluorophenylmethyl)-N-(1-methylpiperidin-4-yl)-N′-(4-(2-methylpropyloxy)phenylmethyl)carbamide or a pharmaceutically acceptable salt thereof. Also provided herein is a method of treating depression in a patient in need thereof, wherein the patient is also suffering from Parkinson's disease, comprising orally administering to the patient 34 mg of N-(4-fluorophenylmethyl)-N-1-methylpiperidin-4-yl)-N′-(4-(2-methylpropyloxy)phenylmethyl)carbamide once daily, as well as methods for the treatment of a SSRI or SNRI-induced sexual dysfunction in a human subject.

### CROSS REFERENCE TO RELATED APPLICATIONS

This application claims priority to and the benefit of U.S. Provisional Patent Application No. 62/752,990, filed Oct. 30, 2018, 62/812,366 filed Mar. 1, 2019, 62/837,915 filed Apr. 24, 2019, 62/856,312 filed Jun. 3, 2019, and 62/905,708 filed Sep. 25, 2019.

### BACKGROUND

Depression may refer to a syndrome or a specific mental disorder. Specific mood disorders that cause clinical depression include major depressive disorder (MDD), persistent depressive disorder (PDD), and disruptive mood dysregulation disorder (DMDD).

Depression can occur for a variety of reasons; however, the exact causes of depression have not been known. MDD, the most common form of depression, is characterized by a symptom of an overwhelming feeling of sadness or a loss of interest and pleasure in most usual activities. (Kessler R C, Chiu W T, Demler O, Merikangas K R, Walters E E. Arch Gen Psychiatry. 2005; 62(6):617-627.) Other symptoms associated with MDD include decrease or increase in appetite, insomnia or hypersomnia, psycho motor agitation or retardation, constant fatigue, feelings of worthlessness or excessive and inappropriate guilt, recurrent thoughts of death and suicidal ideation with or without specific plans for committing suicide, and cognitive difficulties. Depression is ranked as the second leading cause of disability worldwide by the World Health Organization (Murray C J, Lopez A D. N. Engl. J. Med. 2013, 369(5), 448). MDD is a serious, often recurrent medical condition associated with an average lifetime incidence of DSM-4 major depressive episodes of 14.6% and a lifetime risk of suicide attempt of 15.9% (Chen Y W and Dilsaver S C. Biol Psychiatry. 1996, 39(10), 896; Bromet et al. BMC Med. 2011, 9:90).

Treatment of depression may include psychotherapy, pharmacotherapy, or a combination. Pharmacotherapy of depression usually includes prescription of antidepressant drugs, including, for example, selective serotonin reuptake inhibitors (SSRIs) and serotonin-norepinephrine reuptake inhibitors (SNRIs). Antipsychotics can be used as adjunct treatment of depression but are limited due to potential treatment-limiting toxicities, including movement disorders, metabolic effects, weight gain, motor disorders, and sedation. Thus, while some medications have demonstrated modest efficacy, improvements are needed in both their efficacy and their safety and tolerability profiles.

Sexual dysfunction commonly occurs in both treated and untreated subjects with MDD. About 27-65% of female and 26-57% of male patients may experience either a worsening of, or the emergence of new sexual dysfunction, with antidepressant treatment (Baldwin et al., 2013, Depress. Res. Treat., 2013:256841). Antidepressant drugs such as SSRIs and SNRIs vary in potential to cause sexual dysfunction. Baldwin et al (2013) report that most antidepressants contribute to sexual dysfunction and that among approved SSRI/SNRI medications the odds ratio (drug to placebo) for sexual dysfunction may range from 3.27 to 1 among patients treated with fluvoxamine, an SSRI, to 24.42 to 1 among subjects treated with venlafaxine, an SNRI.

N-(4-Fluorophenylmethyl)-N-(1-methylpiperidin-4-yl)-N′-(4-(2-methylpropyloxy)phenylmethyl)carbamide), also known as pimavanserin, is a selective serotonin 5-HT_{2A} inverse agonist and has been approved by U.S. Food and Drug Administration (FDA) in treating hallucinations and delusions associated with Parkinson's disease.

### SUMMARY

The disclosure provides, in part, a method of treating anxious distress with major depressive disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof. Also provided herein are methods of treatment of depression and related symptoms in a patient in need thereof, where the patient is also suffering from Parkinson's disease, and the method includes orally administering to the patient N-(4-fluorophenylmethyl)-N-(1-methylpiperidin-4-yl)-N′-(4-(2-methylpropyloxy)phenylmethyl)carbamide as monotherapy or in combination with antidepressants. Methods provided herein include methods for treating SSRI or SNRI induced sexual dysfunction in a subject with SSRI or SNRI induced sexual dysfunction, in which the methods comprise administering N-(4-fluorophenylmethyl)-N-(1-methylpiperidin-4-yl)-N′-(4-(2-methylpropyloxy)phenylmethyl)carbamide, or a pharmaceutically acceptable salt thereof, to the subject.

In one aspect, the present disclosure provides a method of treating major depressive disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof. In some embodiments, the patient also suffers from anxious distress.

In certain embodiments, after 5 weeks of daily administration, the patient has an improved score relative to the baseline in one or more of the Hamilton Depression Scale individual items selected from the group consisting of psychic anxiety, somatic anxiety, gastrointestinal symptoms, general somatic symptoms, work and interest, and hypochondriasis.

Also provided herein is a method of treating treatment-resistant major depressive disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

In some embodiments, the patient has a DSM-5 defined diagnosis of major depressive disorder. In other embodiments, the patient is being treated with an SSRI or SNRI. In certain embodiments, the patient has an inadequate response to the SSRI or SNRI alone. In some embodiments, the patient has a 50% or greater improvement in a Hamilton Depression Scale 17-item (HAMD-17) score relative to the baseline.

In another aspect, provided herein is a method of treating melancholic features with major depressive disorder in a patient in need thereof, comprising orally administering daily to the patient a compound selected from the group consisting of N-(4-fluorophenylmethyl)-N-(1-methylpiperidin-4-yl)-N′-(4-(2-methylpropyloxy)phenylmethyl)carbamide and pharmaceutically acceptable salts thereof.

Methods provided herein include a method of treating generalized anxiety disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

Another contemplated method provided herein is a method of treating obsessive compulsive disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof, for example, wherein the patient is not or has not been responsive or has inadequate response to treatment with an SSRI and/or a SNRI alone. For example, such methods may include administering N-(4-fluorophenylmethyl)-N-(1-methylpiperidin-4-yl)-N′-(4-(2-methylpropyloxy)phenylmethyl)carbamide or a pharmaceutically acceptable salt thereof and an SSRI (and/or a SNRI) to the patient.

The present disclosure also provides a method of treating post-traumatic stress disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof for example, wherein the patient is not or has not been responsive or has inadequate response to treatment with an SSRI and/or a SNRI alone. For example, such methods may include administering N-(4-fluorophenylmethyl)-N-(1-methylpiperidin-4-yl)-N′-(4-(2-methylpropyloxy)phenylmethyl)carbamide or a pharmaceutically acceptable salt thereof and an SSRI (and/or a SNRI) to the patient

In another aspect, the present disclosure provides method of treating a somatic symptom disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

Also provided herein is a method of treating an illness anxiety disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

In some embodiments, orally administering comprises administering about 5 mg to about 40 mg (e.g., 34 mg), based on the free base form, of N-(4-fluorophenylmethyl)-N-(1-methylpiperidin-4-yl)-N′-(4-(2-methylpropyloxy)phenylmethyl)carbamide daily.

Methods provided herein includes a method of treating anxious distress in a patient diagnosed with depression, comprising orally administering about 34 mg, once daily to the patient an effective amount of N-(4-fluorophenylmethyl)-N-(1-methylpiperidin-4-yl)-N′-(4-(2-methylpropyloxy)phenylmethyl)carbamide.

In one aspect, the present disclosure provides a method of treating depression in a patient in need thereof, where the patient is also suffering from Parkinson's disease, comprising: orally administering to the patient 34 mg of N-(4-fluorophenylmethyl)-N-(1-methylpiperidin-4-yl)-N′-(4-(2-methylpropyloxy)phenylmethyl)carbamide once daily. N-(4-fluorophenylmethyl)-N-(1-methylpiperidin-4-yl)-N′-(4-(2-methylpropyloxy)phenylmethyl)carbamide may be administered, for example, in the form of a pharmaceutically acceptable salt such as a tartrate salt.

Methods provided herein, may provide to a patient, in some embodiments, after 8 weeks of administration of a disclosed compound as monotherapy or in combination with antidepressants such as SSRIs, an improvement in depression symptoms such as measured by the Hamilton Depression Scale. For example, in certain embodiments, contemplated methods of treating may result in the patient having a HAMD-17 score of less than or equal to 7, for example, after 1 or more weeks (e.g. 8 weeks) of administration of the compound.

In another aspect, the present disclosure provides a method of treating depression associated with Parkinson's disease in an adult patient in need thereof including administering 34 mg of N-(4-fluorophenylmethyl)-N-(1-methylpiperidin-4-yl)-N′-(4-(2-methylpropyloxy)phenylmethyl)carbamide.

Also provided herein are methods for treating sexual dysfunction in a human subject with major depressive disorder comprising administering pimavanserin, or a pharmaceutically acceptable salt thereof, to the human subject.

The present disclosure also provides a method for the treatment of major depressive disorder comprising the administration of a therapeutically effective amount of pimavanserin or a pharmaceutically acceptable salt thereof to a patient in need thereof, wherein said patient has previously received a SSRI or a SRNI or is concurrently also being administered a SSRI or a SRNI for the treatment of the major depressive disorder and the SSRI or the SRNI has caused sexual dysfunction.

In certain embodiments of methods provided herein the pimavanserin or pharmaceutically acceptable salt thereof is administered orally to the human subject. In some embodiments a tartrate salt of pimavanserin is administered to the human subject.

The pimavanserin or pharmaceutically acceptable salt thereof can, for example, be administered once, twice or three times per day. The pimavanserin or pharmaceutically acceptable salt thereof can, for example, be administered in a daily dose between 0.5 mg to 120 mg, or between 8 mg to 42 mg.

In some embodiments, the human subject with MDD is taking a SSRI or SNRI to treat MDD.

In some embodiments of the methods provided herein, the human subject is taking an SSRI and/or SNRI antidepressant such as, for example, citalopram, escitalopram (LEXAPRO), fluoxetine (PROZAC), fluvoxamine, paroxetine (PAXIL), sertraline, vilazodone, vortioxetine, desvenlafaxine, duloxetine, levomilnacipran, sibutramine, tramadol, milnacipran or venlafaxine.

In certain embodiments, the pimavanserin or pharmaceutically acceptable salt thereof is administered daily to the human subject for at least 1, 2, 3, 4, 5, 6, 7, 8, 9 or 10 or more weeks.

In some embodiments the methods provided herein further comprise assessing sexual dysfunction in the human subject. Sexual dysfunction can be assessed, for instance, using the Massachusetts General Hospital Sexual Functioning Index (MGH-SFI). In certain embodiments, the human subject's MGH-SFI score in a functional domain after the administration of the pimavanserin or pharmaceutically acceptable salt thereof is reduced as compared to the human subject's MGH-SFI score in the functional domain. For instance, the reduction in MGH-SFI score in the functional domain can, for example, 1, 2, 3, 4 or 5 points using a 6-point scale.

In some embodiments, the functional domain in MGH-SFI is selected from the group consisting of “interest in sex,” “sexual arousal,” “ability to achieve orgasm,” “ability to maintain erection” [males only], and “sexual satisfaction”. The reduction in MGH-SFI score can, for example, occur in 1, 2, 3 or 4 domains of the MGH-SFI. For example, the reduction in MGH-SFI score may occur after daily administration of the pimavanserin or pharmaceutically acceptable salt thereof for at least 2 days, 3 days, 4 days, 5 days, 6 days, 7 days, 8 days, 9 days, 10 days, 11 days, 12 days, 13 days or 14 or more days. A subject may have a score of 3, 4, 5 or 6 in a 6-point scale in one, two, three or four domains of the MGH-SFI prior to administration of pimavanserin, or a pharmaceutically acceptable salt thereof.

Also provided herein are methods of reducing sexual dysfunction in a human subject on antidepressant therapy comprising administering pimavanserin, or a pharmaceutically acceptable salt thereof, to the human subject. Sexual dysfunctions can, for example, be a loss of interest in sex, loss of sexual arousal, diminished ability or inability to achieve orgasm, loss of ability to maintain an erection in male subjects, loss of sexual satisfaction, reduced frequency of sexual activity, or reduced satisfaction from sexual activity.

### BRIEF DESCRIPTION OF THE DRAWINGS

FIG. 1 shows LSMean change from baseline to Week 5 for the HAMD-17 total score.

FIG. 2 shows LSMean change from baseline to Week 5 for the Sheehan Disability Score (SDS).

FIG. 3 shows the response and remission rate with pimavanserin or placebo during Stage 1.

FIG. 4 shows LSMean change from baseline to Week 10 for HAMD-17 total score.

FIG. 5 shows LSMean change from baseline to Week 10 for the SDS.

FIG. 6 shows HAMD-17 total score change in trial Stage 1 among subjects with anxious distress at baseline.

FIG. 7 shows HAMD-17 “anxiety somatization” item score change in trial Stage 1.

FIG. 8 shows HAMD-17 “anxiety somatization” symptoms score change among the subgroup of subjects with higher anxious distress in trial Stage 1.

FIG. 9 shows time to early improvement in Stage 1 with pimavanserin or placebo.

FIG. 10 shows effect size and 95% CI for HAMD-17 items at Stage 1 Week 5 of trial.

FIG. 11 shows the mean change in HAMD-17 individual item scores from baseline to Week 5 with pimavanserin or placebo.

FIG. 12 shows HAMD-17 melancholic symptom subscale score change in trial Stage 1.

FIG. 13 shows functional remission rate by visit (week) with placebo or pimavanserin in Stage 1.

FIG. 14 shows SDS individual items “work/school,” “social life,” “family life/home responsibilities” score change in Stage 1.

FIG. 15 shows SDS item “days unproductive” changes from baseline by visit in Stage 1.

FIG. 16 shows a summary of observed treatment effect sizes for pimavanserin arm compared to placebo arm (black boxes) and 95% confidence intervals (CI) on individual items of Massachusetts General Hospital Sexual Functioning Index (MGH-SFI) in the overall study group, subgroup of <=50 and subgroup of >50, respectively. A positive values on the x-axis indicates that the pimavanserin arm performs better than the placebo arm on the MGH-SFI items. A negative values on the x-axis indicates that the placebo arm performs better than the pimavanserin arm. The effect size of value 0 (dotted line in the middle) represents that there is no difference between the pimavanserin arm and the placebo arm.

FIG. 17 shows a summary of observed treatment effect sizes for pimavanserin arm compared to placebo arm (black boxes) and 95% confidence intervals (CI) on individual items of Massachusetts General Hospital Sexual Functioning Index (MGH-SFI) in the overall study group, subgroup of female and subgroup of male, respectively. A positive values on the x-axis indicates that the pimavanserin arm performs better than the placebo arm on the MGH-SFI items. A negative values on the x-axis indicates that the placebo arm performs better than the pimavanserin arm. The effect size of value 0 (dotted line in the middle) represents that there is no difference between the pimavanserin arm and the placebo arm.

FIG. 18 shows LS Mean (SE) change from baseline to Week 5 for individual items of the MGH-SFI (overall study group).

FIG. 19 shows LS Mean (SE) change in MGH-SFI item scores when stratified by age (>50 vs≤50 Years).

FIG. 20 shows LS Mean (SE) change in MGH-SFI item scores when stratified by sex.

FIG. 21 shows LS Mean (SE) change from baseline to week 5 for MGH-SFI Mean Score A) among patients with or without remission during Stage 1 only; and B) among patients with or without response during Stage 1 only.

FIG. 22 shows LS Mean (SE) change from baseline to week 5 for HAMD-17 item 14 (Sexual Interest).

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

In the present specification, the term “therapeutically effective amount” means the amount of the subject compound that will elicit the biological or medical response of a tissue, system, animal or human that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect, such as an amount which results in the treatment of depression.

“Hamilton Depression Scale 17-item (HAMD-17)” is a multiple item questionnaire used to provide an indication of depression, and is known to those skilled in the art. The questionnaire is designed for adults to rate the severity of depression by probing mood, feelings of guilt, suicide ideation, insomnia, work and interests, agitation or retardation, anxiety, weight loss, and somatic symptoms. Each item on the questionnaire is scored on a 3 or 5 point scale, depending on the item, and the total score is compared to the corresponding descriptor. A score of 0-7 is considered to be normal. Scores of 14 or higher indicate moderate, severe, or very severe depression. Assessment time is estimated at 20 minutes. (See Guy W. Clinical Global Impressions. In: ECDEU Assessment Manual for Psychopharmacology—Revised (DHEW publication number ADM 76-338). Rockville, Md.: US Department of Health, Education, and Welfare. 1976; 218-222; Hamilton M. J. Neurol. Neurosurg. Psychiat. 1960(23):56; Kind P. The EuroQol instrument: An index of health related quality of life. In: Spiker B, ed. Quality of Life and Pharmaco Economics in Clinical Trials. 2nd ed. Philadelphia, Pa.: Lippincott-Raven Publishers; 1996.)

“Hamilton Depression Scale 6-item (HAMD-6)” is a subscale derived from the 17-item Hamilton Rating Scale for Depression (HAMD-17). HAMD-6 includes the following items: depressed mood, guilt feelings, work and interests, psychomotor retardation, psychic anxiety, and general somatic symptoms (Bech et al. Acta Psychiatr Scand. 1975, 51(3):161-70).

“Montgomery-Åsberg Depression Rating Scale (MADRS)” is a clinical rating scale for depression (Montgomery and Åsberg, Br J Psychiatry. 1979; 134:382-389) and includes the following 10 items: 1) apparent sadness; 2) reported sadness; 3) inner tension; 4) reduced sleep; 5) reduced appetite; 6) concentration difficulties; 7) lassitude; 8) inability to feel; 9) pessimistic thoughts; and 10) suicidal thoughts. MADRS items are rated on a 0-6 continuum (0=no abnormality, 6=severe).

“Clinical Global Impression (CGI)” scale is a clinician-rated, 7-point scale that is designed to rate the severity of the subject's depression at the time of assessment using the Investigator's judgment and past experience with subjects who have the same disorder (i.e., depression with anxious distress). “Clinical Global Impression-Improvement (CGI-I)” is a clinician-rated, 7-point scale that is designed to rate the improvement in the subject's depression at the time of assessment, relative to the symptoms at Baseline. “Clinical Global Impression—Severity (CGI-S)” is an assessment of the current severity of the patient's disease.

“Sheehan Disability Score (SDS)” is used to assess functional disability and improvement in workplace function has been demonstrated with antidepressant therapy in patients with MDD (Lee et al, J Affect Disord. 2018; 227:406-415). The SDS is well validated and widely accepted for assessing functional outcomes in patients with MDD (Weiller et al, Neuropsychiatr Dis Treat. 2017; 14:103-115). In a systematic review of studies that assessed functional outcomes, the authors suggested that improvements in function (SDS) should be considered for inclusion as co-endpoints with symptomatic assessments when evaluating treatments for MDD (Sheehan et al, J Affect Disord. 2017; 215:299-313). Routine assessments of both symptoms and function could be helpful for minimizing residual effects that increase the risk for relapse or recurrence (Sheehan et al, J Affect Disord. 2017; 215:299-313).

“Functional remission” as used herein refers to remission in functional impairment. Functional remission is defined as a SDS total score of 6 or less at endpoint. (Sheehan et al. Human Psychopharmacology 2016, 31, 53-63).

“Karolinska Sleepiness Scale (KSS)” is a scale for evaluating subjective sleepiness (Kaida et al. Clinical Neurophysiology 2006, 117, 7, 1574-1581).

“The 12-Item Short-Form Health Survey (SH-12)” (Ware J E Jr et al. Med Care. 1996; 34(3):220-233) is a 12 item-questionnaire derived from the Medical Outcomes Study 36-Item Short-Form Health Survey (SF-36).

As used herein, “treatment-resistant major depressive disorder” refers to major depressive disorder where the patient had a history of an inadequate response when administered 1 or 2 other antidepressant treatments (e.g., administered an SSRI or SNRI) during e.g., a current depression episode. In some embodiments, the patient had a history of an inadequate response when administered an SSRI and SNRI.

“Inadequate response” to an antidepressant (e.g., an SSRI or SNRI) may be determined through the administration of the MGH Antidepressant Treatment Response Questionnaire (ATRQ), which is a self-rated scale used to determine treatment resistance in major depressive disorder (Chandler et al. CNS Neurosci Ther. 2010; 16(5):322-325).

“Early improvement as measured by Hamilton Depression Scale,” as used herein, refers to a reduction from baseline in HAMD-17 total score of 20% or more.

A patient with “DMS-5 defined diagnosis of major depressive disorder” has 5 or more of the symptoms as described in Diagnostic and Statistical Manual of Mental Disorders, Fifth Edition (DSM-5) during a 2-week period and at least one of the symptoms is either 1) depressed mood or 2) loss of interest or pleasure. The symptoms as described in DMS-5 include the following: 1) depressed mood; 2) loss of interest or pleasure; 3) significant weight loss; 4) insomnia or hypersomnia; 5) psychomotor agitation or retardation; 6) fatigue or loss of energy; 7) feelings of worthlessness or excessive or inappropriate guilt; 8) diminished ability to think or concentrate or indecisiveness; and 9) recurrent thoughts of death.

“Scale of Outcomes in Parkinson's Disease (SCOPA) Sleep” is a scale developed for research in Parkinson disease to evaluate nighttime sleep and daytime sleepiness and has high internal consistency for the nighttime sleep and daytime sleepiness scales (0.88 and 0.91, respectively), and test-retest reliabilities (0.94 and 0.89, respectively). Scores on the SCOPA Sleep Scale show high correlations between the nighttime sleep scale and the Pittsburgh Sleep Quality Index (0.83), and between the daytime sleepiness scale and the Epworth Sleepiness Scale (0.81). The coefficient of variation of both the nighttime sleep and the daytime sleepiness scale can be higher than that of the Pittsburgh Sleep Quality Index and the Epworth Sleepiness Scale, indicating a better ability to detect differences between individuals. (Marinus J, Visser M, van Hilten J J, Lammers G J, Stiggelbout A M. Sleep. 2003; 26(8):1049-1054.)

“EuroQol-5 dimensions-5 level (EQ-5D-5L)” is a standardized instrument used as a measure of health outcome and measures 5 dimensions (mobility, self-care, usual activities, pain/discomfort, anxiety/depression), each of which has 5 potential responses. The responses record 5 levels of severity (no problems/slight problems/moderate problems/severe problems/extreme problems) within a particular EQ-5D dimension.

“Massachusetts General Hospital Sexual Functioning Index (MGH-SFI)” is described, for instance, in Labbate and Lare, 2001, Psychother Psychosom. 70(4):221-225. This is a patient facing questionnaire that quantifies sexual dysfunction into 5 functional domains (“interest in sex,” “sexual arousal,” “ability to achieve orgasm,” “ability to maintain erection” (males only), and “sexual satisfaction”). Patients rate each item using a 6-point scale ranging from 1 (good function) to 6 (poor function). High scores indicate poor sexual functioning.

### Methods

Provided herein, in part, is a method of treating major depressive disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof. Contemplated patients suffering from major depressive disorder may also be suffering from anxious distress.

Contemplated herein is, a method of treating anxious distress with major depressive disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof. For example, in methods of treatment contemplated herein, after 5 weeks of daily administration of a N-(4-fluorophenylmethyl)-N-(1-methylpiperidin-4-yl)-N′-(4-(2-methylpropyloxy)phenylmethyl)carbamide or a pharmaceutically acceptable salt thereof, the patient may have an improved score relative to the baseline in one or more of the Hamilton Depression Scale individual items selected from the group consisting of psychic anxiety, somatic anxiety, gastrointestinal symptoms, general somatic symptoms, work and interest, and hypochondriasis. Such a disclosed method may improve one or more of: feelings of restlessness, tension, loss of control, and/or improve concentration. Contemplated patients may also be concurrently or continuing treatment with one or more other depression treatments, e.g., may be concurrently or continuing treatment with an SSRI or SNRI.

The present disclosure provides, in a particular embodiment, a method of treating treatment-resistant major depressive disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

Contemplated patients that may be treating using disclosed methods may have a DSM-5 defined diagnosis of major depressive disorder and/or may have a history of MDD diagnosis at least 1 year prior to the screening.

In other embodiments, a patient has anxious distress during a majority of days of a major depressive disorder episode. In some embodiments, the anxiety distress is selected from any one of the group consisting of separation anxiety disorder, selective mutism, specific phobia, social anxiety disorder, panic disorder, panic attack specifier, agoraphobia, generalized anxiety disorder, substance/medication-induced anxiety disorder, anxiety disorder due to another medical condition, post-traumatic stress disorder, and obsessive compulsive disorder.

For example, the anxious distress is generalized anxiety disorder and the patient is being treated with paroxetine, escitalopram, duloxetine, or venlafaxine. For example, the anxiety distress is post-traumatic stress disorder and the patient is being treated with sertraline or paroxetine. For example, the anxiety distress is obsessive compulsive disorder and the patient is being treated with fluoxetine, fluvoxamine, paroxetine or sertraline. For example, the anxiety distress is panic disorder, and the patient is being treated with fluoxetine, paroxetine, sertraline, or venlafaxine.

In some embodiments, a contemplated patient that may be treated by disclosed methods is a patient is being treated with an SSRI or SNRI. For example, a contemplated patient may have been treated with SSRI or SNRI for at least 8 weeks, for at least 12 weeks, or for at least 16 weeks, or at least 20 weeks during the current depression episode. In other embodiments, the patient has been treated with the same dose of the SSRI or SNRI for at least 4 weeks, at least 8 weeks, at least 12 weeks, or at least 16 weeks during the current depression episode.

A contemplated patient may have an inadequate response to the SSRI or SNRI alone. For example, a contemplated patient may have had a history of inadequate response to 1 or 2 antidepressant treatments during the current depression episode. For example, the inadequate response may be determined by MGH ATRQ (Antidepressant Treatment Response Questionnaire). For example, the SSRI or SNRI is selected from the group consisting of: citalopram, escitalopram, paroxetine, fluoxetine, sertraline, duloxetine, venlafaxine, and desvenlafaxine, and fluvoxamine. For example, the SSRI is paroxetine.

A contemplated patient may be 18 years or older. In some embodiments, a contemplated patient may have had at least one year of history of major depression disorder. In certain embodiments, a patient does not have a psychotic disorder other than MDD. In certain embodiments, the patient does not have a history or symptoms of long QT syndrome.

In certain embodiments, a contemplated patient has a MADRS (Montgomery-Åsberg Depression Rating Scale) score of at least 20, at least 22, or at least 24 before the administration (e.g., at screening, at baseline). For example, the patient may have a MADRS score of at least 20 before the administration. A contemplated patient may have a CGI-S score of at least 3, at least 4 or at least 5 before the administration.

After 5 weeks of daily administration to a patient of N-(4-fluorophenylmethyl)-N-(1-methylpiperidin-4-yl)-N′-(4-(2-methylpropyloxy)phenylmethyl)carbamide or a pharmaceutically acceptable salt thereof as described herein, a patient may have a 50% or greater improvement in a HAMD-17 score relative to the baseline. For example, after 5 weeks of daily administration, the patient may have a 30% or greater, 40% or greater, 60% or greater, 70% or greater, or 80% or greater improvement in a HAMD-17 score relative to the baseline. For example, after 5 weeks of daily administration, the patient may have a HAMD-17 score of less than 14. For example, after 5 weeks of daily administration, the patient may have a HAMD-17 score of less than 20, less than 19, less than 18, less than 17, less than 16, less than 15, less than 13, less than 12, less than 10, less than 9, or less than 8.

In some embodiments, after 5 weeks of daily administration, the patient has an improved Sheehan Disability Score (SDS) relative to the baseline.

In certain embodiments, the treating results in increased functional remission compared to a patient treated with a placebo. In some embodiments, the functional remission is a total SDS score of 6 or less.

In other embodiments, after 5 weeks of daily administration, the patient has an improved score in the work/school item of the SDS.

In certain embodiments, after 5 weeks of daily administration, the patient has an improved score in the social life item of the SDS.

In some embodiments, after 5 weeks of daily administration, the patient has an improved score in the family life/home responsibilities item of the SDS.

In certain embodiments, after 5 weeks of daily administration, the patient has a reduced number of the days unproductive item of the SDS.

In certain embodiments, after 5 weeks of daily administration, the patient has a higher improvement in a HAMD-17 score than a patient treated with an SSRI or SNRI alone. For example, the patient may have an early improvement as measured by Hamilton Depression Scale within 7 days of daily administration compared to a patient treated with a placebo. For example, the patient may have an early improvement as measured by Hamilton Depression Scale within 8 days of daily administration compared to a patient treated with a placebo. For example, the patient may have an early improvement as measured by Hamilton Depression Scale within 9 days of daily administration compared to a patient treated with a placebo. For example, the patient may have an early improvement as measured by Hamilton Depression Scale within 10 days of daily administration compared to a patient treated with a placebo.

For example, after 5 weeks of daily administration, the patient may have an improved score in the psychic anxiety item of the Hamilton Depression Scale. For example, after 5 weeks of daily administration, the patient has an improved score in the somatic anxiety item of the Hamilton Depression Scale. For example, after 5 weeks of daily administration, the patient has an improved score in the work and interest item of the Hamilton Depression Scale. For example, after 5 weeks of daily administration, the patient has an improved score in the gastrointestinal symptoms item of the Hamilton Depression Scale. For example, after 5 weeks of daily administration, the patient has an improved score in the general somatic symptoms item of the Hamilton Depression Scale. For example, the patient has an improved score in the hypochondria item of the Hamilton Depression Scale.

Provided herein, for example, is a method of increasing work and interest, in a patient having depression, for example as measured by the Hamilton Depression Scale.

Also provided herein is a method of treating melancholic features with major depressive disorder in a patient in need thereof, comprising orally administering daily to the patient a compound selected from the group consisting of N-(4-fluorophenylmethyl)-N-(1-methylpiperidin-4-yl)-N′-(4-(2-methylpropyloxy)phenylmethyl)carbamide and pharmaceutically acceptable salts thereof. In an embodiment, a contemplated patient, before administration of N-(4-fluorophenylmethyl)-N-(1-methylpiperidin-4-yl)-N′-(4-(2-methylpropyloxy)phenylmethyl)carbamide, may wake up 2 hours earlier than usual. In certain embodiments, before administration of N-(4-fluorophenylmethyl)-N-(1-methylpiperidin-4-yl)-N′-(4-(2-methylpropyloxy)phenylmethyl)carbamide, the patient has observable psychomotor retardation or agitation. In other embodiments, before the administration of N-(4-fluorophenylmethyl)-N-(1-methylpiperidin-4-yl)-N′-(4-(2-methylpropyloxy)phenylmethyl)carbamide, the patient has significant weight loss or anorexia. For example, such a disclosed method may improve, in a patient suffering from major depressive disorder, one or more of: depression that is worse in the morning, waking up 2 or more hours earlier than usual, psychomotor retardation or agitation, weight loss or anorexia, and excessive or inappropriate guilt.

In some embodiments, the patient is also being treated with an SSRI or SNRI. For example, the SSRI or SNRI may be selected from the group consisting of: citalopram, escitalopram, paroxetine, fluoxetine, sertraline, duloxetine, venlafaxine, desvenlafaxine, and fluvoxamine.

In other embodiments, the patient may have had an inadequate response to SSRI or SNRI treatment alone.

In contemplated methods, after 5 weeks of daily administration, the patient may have a 50% or greater improvement in a HAMD-6 score relative to the baseline, where the HAMD-6 consists of the following individual items: depressed mood, guilt feelings, work and interests, psychomotor retardation, psychic anxiety, and general somatic symptoms.

For example, after 5 weeks of daily administration, the patient may have a higher improvement in a HAMD-6 score than a patient treated with an SSRI or SNRI alone, wherein the HAMD-6 consists of the following individual items: depressed mood, guilt feelings, work and interests, psychomotor retardation, psychic anxiety, and general somatic symptoms.

For example, after 5 weeks of daily administration, the patient has an improved score in the depressed mood item of the Hamilton Depression Scale. For example, after 5 weeks of daily administration, the patient has an improved score in the guilt item of the Hamilton Depression Scale. For example, after 5 weeks of daily administration, the patient has an improved score in the work and interest item of the Hamilton Depression Scale. For example, after 5 weeks of daily administration, the patient has an improved score in the psychomotor retardation item of the Hamilton Depression Scale. For example. after 5 weeks of daily administration, the patient has an improved score in the gastrointestinal symptoms item of the Hamilton Depression Scale.

In some embodiments, after 5 weeks of daily administration, the patient has improved daytime sleepiness and/or improved nighttime sleep disturbance. For example, the improved sleep disturbance is measured by at least a −1.5 change from baseline of the Karolinska Sleepiness Scale.

In another aspect, provided herein is a method of treating generalized anxiety disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

Another contemplated method provided herein is a method of treating obsessive compulsive disorder and/or post-traumatic stress disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof. For example, wherein the patient is not or has not been responsive or has inadequate response to treatment with an SSRI and/or a SNRI alone. For example, such methods may include administering N-(4-fluorophenylmethyl)-N-(1-methylpiperidin-4-yl)-N′-(4-(2-methylpropyloxy)phenylmethyl)carbamide or a pharmaceutically acceptable salt thereof and an SSRI (and/or a SNRI) to the patient.

Also provided herein is a method of treating a somatic symptom disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

The methods provided in the present disclosure includes a method of treating an illness anxiety disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

In some embodiments, the patient is also being treated with an SSRI or SNRI. For example, the SSRI or SNRI may be selected from the group consisting of: citalopram, escitalopram, paroxetine, fluoxetine, sertraline, duloxetine, venlafaxine, and desvenlafaxine. Such contemplated patients may have had an inadequate response to SSRI or SNRI treatment alone.

In some embodiments, after 5 weeks of daily administration, the patient has an improved score in the psychic anxiety item of the Hamilton Depression Scale. In certain embodiments, after 5 weeks of daily administration, the patient has an improved score in the somatic anxiety item of the Hamilton Depression Scale. In other embodiments, after 5 weeks of daily administration, the patient has an improved score in the work and interest item of the Hamilton Depression Scale.

In some embodiments, N-(4-fluorophenylmethyl)-N-(1-methylpiperidin-4-yl)-N′-(4-(2-methylpropyloxy)phenylmethyl)carbamide is administered in the form of a tartrate salt.

In certain embodiments, orally administering comprises administering about 5 mg to about 40 mg, based on the free base form, of N-(4-fluorophenylmethyl)-N-(1-methylpiperidin-4-yl)-N′-(4-(2-methylpropyloxy)phenylmethyl)carbamide daily.

For example, orally administering may comprise administering 34 mg based on the free base form of N-(4-fluorophenylmethyl)-N-(1-methylpiperidin-4-yl)-N′-(4-(2-methylpropyloxy)phenylmethyl)carbamide daily. In some embodiments, the 34 mg is administered in one capsule or two tablets (2×17 mg tablets).

The contemplated patient may be concurrently being administered a CYP3A4 inhibitor, wherein orally administering comprises administering 10 mg of N-(4-fluorophenylmethyl)-N-(1-methylpiperidin-4-yl)-N′-(4-(2-methylpropyloxy)phenylmethyl)carbamide daily.

In another aspect, the present disclosure provides a method of treating anxious distress in a patient diagnosed with depression, comprising orally administering about 34 mg, once daily to the patient an effective amount of N-(4-fluorophenylmethyl)-N-(1-methylpiperidin-4-yl)-N′-(4-(2-methylpropyloxy)phenylmethyl)carbamide.

In some embodiments, the depression is major depressive disorder. In certain embodiments, the depression is treatment resistant depression.

The present disclosure provides, in part, a method of treating depression in a patient in need thereof, wherein the patient is also suffering from Parkinson's disease, comprising orally administering to the patient 34 mg of the compound N-(4-fluorophenylmethyl)-N-(1-methylpiperidin-4-yl)-N′-(4-(2-methylpropyloxy)phenylmethyl)carbamide (i.e., 34 mg as the free base), for example, once daily, e.g. comprising administering 34 mg of the compound once daily to the patient. In some embodiments, the patient is diagnosed with Parkinson's disease. In certain embodiments, the patient is undergoing treatment for Parkinson's disease. In some embodiments, the patient is suffering from Parkinson's disease psychosis. In some embodiments, after 2, 4, 6, 8 or 10 weeks of administration of the compound, contemplated patients have an improvement in depression symptoms as measured by the Hamilton Depression Scale, e.g., the Hamilton Depression Scale-17 items (HAMD-17).

In another aspect, the present disclosure provides a method of treating depression in a patient in need thereof, wherein the patient is also suffering from Parkinson's disease dementia, comprising orally administering to the patient 34 mg of the compound N-(4-fluorophenylmethyl)-N-(1-methylpiperidin-4-yl)-N′-(4-(2-methylpropyloxy)phenylmethyl)carbamide (i.e., 34 mg as the free base), for example, once daily, e.g. comprising administering 34 mg of the compound once daily to the patient. In some embodiments, the patient is diagnosed with Parkinson's disease dementia. In certain embodiments, the patient is undergoing treatment for Parkinson's disease dementia.

For example a patient contemplated for methods of treating disclosed herein, may have an initial HAMD-17 score before treatment, of more than or equal to 15 at the baseline, or less than or equal to 15. Contemplated treatments can result, in certain embodiments, in the patient having a HAMD-17 score of less than or equal to 10, less than or equal to 9, less than or equal to 8, or less than or equal to 7, for example, in some embodiments, treating results in the patient having a 50% or greater improvement in HAMD-17 score.

In certain embodiments, treating results in the patient having an improved Scale of Outcomes in PD-Sleep (SCOPA) daytime score, and/or results in the patient having an improved Clinical Global Impression-improvement (CGI-I) score from baseline, e.g., results in the patient having a CGI-I score of 1 or 2 (very much improved or much improved), after 8 weeks or more of administration. In certain embodiments, treating results in the patient having an improved quality of life as measured by EQ-5D-5L.

In certain embodiments, a contemplated patient who may be treated using contemplated methods has suffered from Parkinson's disease for one year or more. For example, a contemplated patient may have one or more (e.g. at least 3) of the indications selected from the group consisting of: rest tremor, rigidity, bradykinesia and/or akinesia, and postural and gait abnormalities. Contemplated patients may additionally meet clinical criteria for depression with Parkinson's disease as listed in the NINDS/NIMH guidelines.

A contemplated patient, in certain embodiments, may be also treated (or are being treated) with an antidepressant, for example, an SSRI or SNRI antidepressant, such as citalopram, escitalopram, fluoxetine, fluvoxamine, partoxetine, sertraline, duloxetine, levomilnacipran, milnacipran, sibutramine, tramadol or venlafaxine. In some embodiments, the subject is on a stable dose of anti-Parkinson's medication for 1 month prior to screening. Contemplated patients, in some embodiments may not have personal or family history or symptoms of long QT syndrome.

Patients that may be treating can be for example, 50 years or older.

In some embodiments, N-(4-fluorophenylmethyl)-N-(1-methylpiperidin-4-yl)-N′-(4-(2-methylpropyloxy)phenylmethyl)carbamide is administered in the form of a capsule.

In some embodiments, N-(4-fluorophenylmethyl)-N-(1-methylpiperidin-4-yl)-N′-(4-(2-methylpropyloxy)phenylmethyl)carbamide is administered in the form of a tablet.

In some embodiments, N-(4-fluorophenylmethyl)-N-(1-methylpiperidin-4-yl)-N′-(4-(2-methylpropyloxy)phenylmethyl)carbamide is administered each day for at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 3 months, 6 months or more.

The compound N-(4-fluorophenylmethyl)-N-(1-methylpiperidin-4-yl)-N′-(4-(2-methylpropyloxy)phenylmethyl)carbamide may be administered in the form of a pharmaceutically acceptable salt such as a tartrate salt.

Contemplated methods include treating depression in a patient in need thereof, where depression may be for example selected may be selected from the group consisting of major depressive disorder, persistent depressive disorder, bipolar disorder, seasonal affective disorder, psychotic depression, peripartum depression, premenstrual dysphoric disorder, situational depression, and atypical depression.

Also provided herein, in part, is a method of treating depression associated with Parkinson's Disease in an adult patient in need thereof comprising administering 34 mg of N-(4-fluorophenylmethyl)-N-(1-methylpiperidin-4-yl)-N′-(4-(2-methylpropyloxy)phenylmethyl)carbamide. The patient may, for example, be concurrently administered an SSRI or SNRI.

In another aspect, provided herein is a method for the treatment of major depressive disorder comprising the administration of a therapeutically effective amount of pimavanserin or a pharmaceutically acceptable salt thereof to a patient in need thereof, wherein said patient has previously received a SSRI or a SRNI or is concurrently also being administered a SSRI or a SRNI for the treatment of the major depressive disorder and the SSRI or the SRNI has caused sexual dysfunction.

In some embodiments, the patient is concurrently being administered the SSRI or the SRNI for the treatment of the major depressive disorder. For example, the contemplated patient may be a male patient. For example, the contemplated patient may be a female patient.

In some embodiments, the sexual dysfunction is loss of interest in sex, loss of sexual arousal, diminished ability or inability to achieve orgasm, loss of ability to maintain an erection in male subjects, loss of sexual satisfaction, reduced frequency of sexual activity, or reduced satisfaction from sexual activity.

Methods provided herein includes a method for the treatment of major depressive disorder comprising the administration of a therapeutically effective amount of pimavanserin or a pharmaceutically acceptable salt thereof to a patient in need thereof, wherein said patient has previously received a SSRI or a SRNI or is concurrently also being administered a SSRI or a SRNI for the treatment of the major depressive disorder and the SSRI or the SRNI has caused gastrointestinal or sleep related adverse events.

In some embodiments, the patient has had an inadequate response to the SSRI or the SNRI alone. In other embodiments, the patient has a DSM-5 defined diagnosis of major depressive disorder. In certain embodiments, the SSRI or SNRI is selected from the group consisting of: citalopram, escitalopram, paroxetine, fluoxetine, sertraline, duloxetine, venlafaxine, desvenlafaxine, and fluvoxamine.

In some embodiments, N-(4-fluorophenylmethyl)-N-(1-methylpiperidin-4-yl)-N′-(4-(2-methylpropyloxy)phenylmethyl)carbamide is administered in the form of a tartrate salt. In other embodiments, orally administering comprises administering about 5 mg to about 40 mg, based on the free base form, of N-(4-fluorophenylmethyl)-N-(1-methylpiperidin-4-yl)-N′-(4-(2-methylpropyloxy)phenylmethyl)carbamide daily. In certain embodiments, orally administering comprises administering 34 mg based on the free base form of N-(4-fluorophenylmethyl)-N-(1-methylpiperidin-4-yl)-N′-(4-(2-methylpropyloxy)phenylmethyl)carbamide daily. In some embodiments, the 34 mg is administered in one capsule or two tablets (2×17 mg tablets). In other embodiments, the patient is concurrently being administered a CYP3A4 inhibitor, wherein orally administering comprises administering 10 mg of N-(4-fluorophenylmethyl)-N-(1-methylpiperidin-4-yl)-N′-(4-(2-methylpropyloxy)phenylmethyl)carbamide daily.

Provided herein are methods for treating selective serotonin reuptake inhibitor (S SRI) or serotonin-norepinephrine reuptake inhibitor (SNRI) induced sexual dysfunction in a human subject with SSRI or SNRI induced sexual dysfunction, in which the methods comprise administering pimavanserin, or a pharmaceutically acceptable salt thereof, to the human subject.

Also provided herein are methods for treating sexual dysfunction in a human subject with major depressive disorder (MDD) comprising administering pimavanserin, or a pharmaceutically acceptable salt thereof, to the human subject.

In certain embodiments of methods provided herein the pimavanserin or pharmaceutically acceptable salt thereof is administered orally to the human subject. In some embodiments a tartrate salt of pimavanserin is administered to the human subject.

The pimavanserin or pharmaceutically acceptable salt thereof can, for example, be administered once, twice or three times per day. The pimavanserin or pharmaceutically acceptable salt thereof can, for example, be administered in a daily dose between 0.5 mg to 120 mg, or between 8 mg to 42 mg, e.g. 10 mg, 20 mg or 34 mg pimavanserin.

In some embodiments, the human subject with MDD is taking a SSRI or SNRI to treat MDD.

In some embodiments of the methods provided herein, the human subject is taking an SSRI and/or SNRI antidepressant such as, for example, citalopram, escitalopram (LEXAPRO), fluoxetine (PROZAC), fluvoxamine, paroxetine (PAXIL), sertraline, vilazodone, vortioxetine, desvenlafaxine, duloxetine, levomilnacipran, sibutramine, tramadol, milnacipran or venlafaxine.

In certain embodiments, the pimavanserin or pharmaceutically acceptable salt thereof is administered daily to the human subject for at least 1, 2, 3, 4, 5, 6, 7, 8, 9 or 10 or more weeks.

In some embodiments the methods provided herein further comprise assessing sexual dysfunction in the human subject. Sexual dysfunction can be assessed, for instance, using the Massachusetts General Hospital Sexual Functioning Index (MGH-SFI). In certain embodiments, the human subject's MGH-SFI score in a functional domain after the administration of the pimavanserin or pharmaceutically acceptable salt thereof is reduced as compared to the human subject's MGH-SFI score in the functional domain. For instance, the reduction in MGH-SFI score in the functional domain can, for example, 1, 2, 3, 4 or 5 points using a 6-point scale.

In some embodiments, the functional domain in MGH-SFI is selected from the group consisting of “interest in sex,” “sexual arousal,” “ability to achieve orgasm,” “ability to maintain erection” [males only], and “sexual satisfaction”. The reduction in MGH-SFI score can, for example, occur in 1, 2, 3 or 4 domains of the MGH-SFI.

In certain embodiments, the reduction in MGH-SFI score occurs after daily administration of the pimavanserin or pharmaceutically acceptable salt thereof for at least 2 days, 3 days, 4 days, 5 days, 6 days, 7 days, 8 days, 9 days, 10 days, 11 days, 12 days, 13 days or 14 or more days.

In certain embodiments of the methods provided the human subject has a score of 3, 4, 5 or 6 in a 6-point scale in one, two, three or four domains of the MGH-SFI prior to administration of pimavanserin, or a pharmaceutically acceptable salt thereof.

Also provided herein are methods of reducing sexual dysfunction in a human subject on antidepressant therapy comprising administering pimavanserin, or a pharmaceutically acceptable salt thereof, to the human subject. The in the reduced sexual dysfunction can, for example, be a loss of interest in sex, loss of sexual arousal, diminished ability or inability to achieve orgasm, loss of ability to maintain an erection in male subjects, loss of sexual satisfaction, reduced frequency of sexual activity, or reduced satisfaction from sexual activity.

Provided herein are methods for the treatment of SSRI or SNRI-induced sexual dysfunction in a subject with SSRI or SNRI induced sexual dysfunction comprising administering a selective serotonin 5-HT_{2A} inverse agonist or antagonist to the subject.

In certain embodiments, SSRI induced sexual dysfunction is treated.

In other embodiments, SNRI induced sexual dysfunction is treated.

Also provided herein are methods for the treatment of sexual dysfunction in a subject with major depressive disorder comprising administering a selective serotonin 5-HT_{2A} inverse agonist or antagonist to the subject.

In certain embodiments the subject with MDD is on an antidepressant therapy.

Methods are also provided herein for reducing a human subject's score on the Massachusetts General Hospital Sexual Functioning Index (MGH-SFI) comprising administering a selective serotonin 5-HT_{2A} inverse agonist or antagonist to the subject.

Also provided herein are methods for reducing sexual dysfunction or improving sexual function in a human subject on an antidepressant therapy.

In the methods described herein, the selective selective serotonin 5-HT_{2A} inverse agonist or antagonist administered to the subject can, for example, be pimavanserin, or a pharmaceutically acceptable salt thereof.

Unless otherwise indicated, a subject can be a mammal, a primate, a monkey or a human. In certain embodiments of the methods provided herein, the subject is a human subject.

In some embodiments, the human subject is a female human subject. In some embodiments, the human subject is a male human subject. In some embodiments, the human subject is a female or male human subject.

In some embodiments, the age of the human subject is 45 years or older, 50 years or older, or 55 years or old, or 60 years or older, or 65 years or older, or 70 years or older at baseline. In another embodiment, the age of the patient is 50 years or older. In another embodiment, the age of the patient is less than 50 years old.

Selective serotonin 5-HT_{2A} inverse agonists or antagonists

Selective serotonin 5-HT_{2A} inverse agonists or antagonists are known in the art (see, e.g., U.S. Pat. No. 6,815,458).

In some embodiments, after 5 weeks of daily administration, the patient has an improved score in the sexual interest item of Hamilton Depression Scale.

In other embodiments, after 2 weeks of daily administration, the patient has an improved score in the sexual interest item of Hamilton Depression Scale.

### Compound

N-(4-Fluorophenylmethyl)-N-(1-methylpiperidin-4-yl)-N′-(4-(2-methylpropyloxy)phenylmethyl)carbamide is also known as pimavanserin; N-[(4-fluorophenyl)methyl]-N-(1-methyl-4-piperidinyl)-N′-[[4-(2-methylpropoxy)phenyl]methyl]-urea, 1-(4-fluorobenzyl)-1-(1-methylpiperidin-4-yl)-3-[4-(2-methylpropoxy)benzyl]urea or ACP-103 and is represented by the chemical formula:

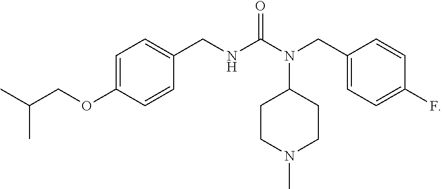 [SMILES: CC(C)COc1ccc(CNC(=O)N(Cc2ccc(F)cc2)C2CCN(C)CC2)cc1]

N-(4-Fluorophenylmethyl)-N-(1-methylpiperidin-4-yl)-N′-(4-(2-methylpropyloxy)phenylmethyl)carbamide maybe administered as a tartrate salt, which is urea, N-[(4-fluorophenyl)methyl]-N-(1-methyl-4-piperidinyl)-N′-[[4-(2-methylpropoxy)phenyl]methyl]-(2R,3R)-2,3-dihydroxybutanedioate (2:1), and represented by the chemical formula:

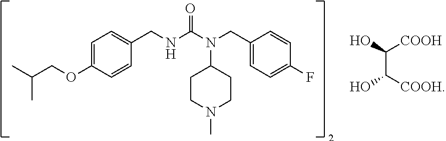

Pimavanserin (i.e., N-(4-Fluorophenylmethyl)-N-(1-methylpiperidin-4-yl)-N′-(4-(2-methylpropyloxy)phenylmethyl)carbamide) can be synthesized according to the method disclosed in Scheme I.

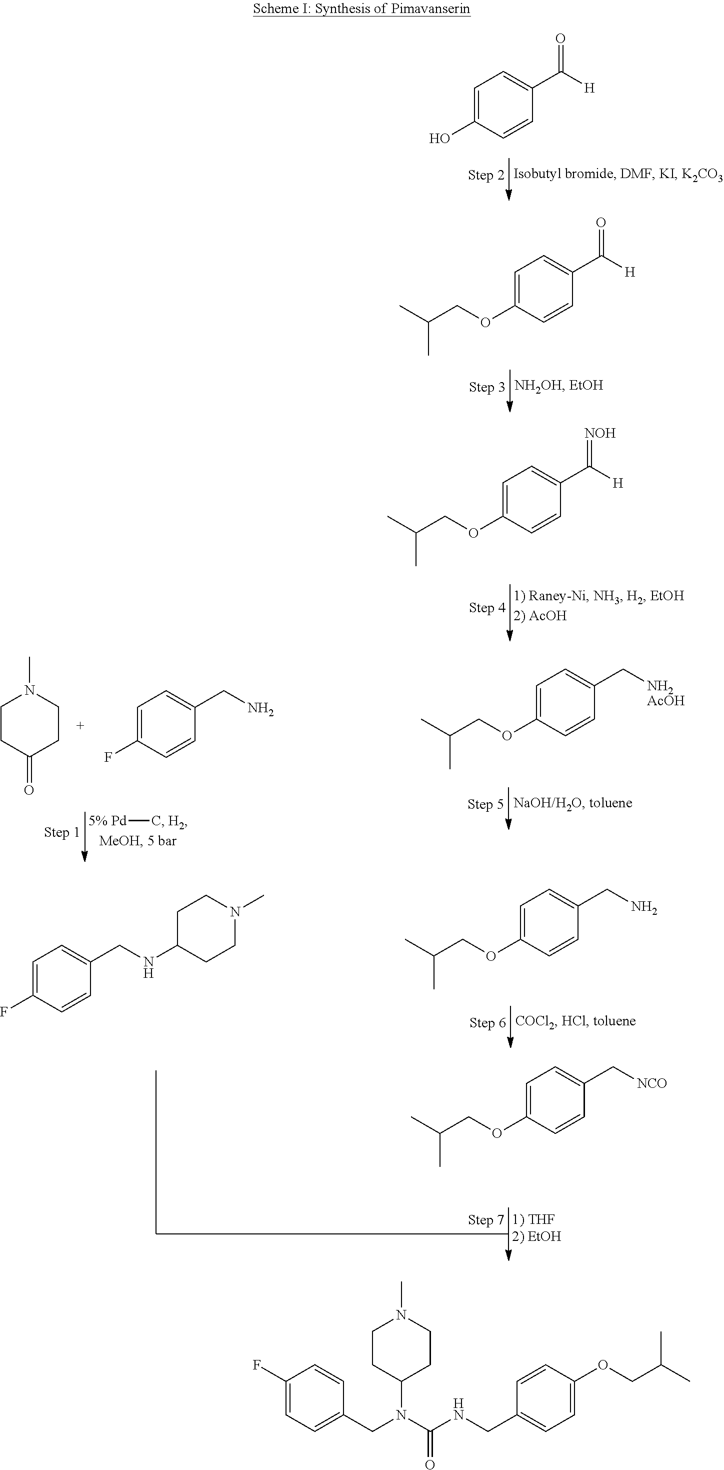

Pimavanserin can be obtained in a number of salt and crystalline forms. Exemplary pharmaceutically acceptable salts include the tartrate, hemi-tartrate, citrate, fumarate, maleate, malate, phosphate, succinate, sulphate, and edisylate (ethanedisulfonate) salts. Pimavanserin, and salts thereof including the aforementioned ions, among others, are described, for example, in International Patent Publication WO 2008/144326, U.S. Patent Publication Nos. 2006-0111399 and 2010-0305329, and International Patent Application WO2017015272, the entirety of which is incorporated herein by reference. In an embodiment provided herein, pimavanserin is the tartrate salt of pimavanserin. Several crystalline forms of the tartrate salt of pimavanserin are known. In an embodiment provided herein, pimavanserin is the crystalline form of the tartrate salt of pimavanserin Form A. In another embodiment, pimavanserin is the crystalline form of the tartrate salt of pimavanserin Form C.

In certain embodiments of the methods provided herein, pimavanserin or a pharmaceutically acceptable salt thereof is administered to the subject in an oral dosage form, such as, for example, a tablet, a capsule, or a lozenge and the like.

In some embodiments, the drug product is formulated with standard pharmaceutical excipients at a 34 mg pimavanserin (40 mg of pimavanserin tartrate) in capsules for oral administration. In certain embodiments the capsule formulation includes inactive ingredients magnesium stearate and microcrystalline cellulose. The following inactive ingredients can, for example, be present as components of the capsule shell: black iron oxide, FD&C blue #1, hypromellose, titanium dioxide, and yellow iron oxide.

In certain embodiments of the methods provided herein, pimavanserin or a pharmaceutically acceptable salt thereof is orally administered to the subject in a capsule, wherein the amount of pimavanserin or pharmaceutically acceptable salt in the capsule is between 2 mg to 80 mg, between 5 mg to 45 mg, or between 9 mg to 42 mg.

In certain embodiments of the methods provided herein, pimavanserin or a pharmaceutically acceptable salt thereof is orally administered to the subject in a tablet, wherein the amount of pimavanserin or pharmaceutically acceptable salt in the tablet is between 2 mg to 80 mg, between 5 mg to 45 mg, or between 9 mg to 42 mg.

Disclosed method include administration of N-(4-fluorophenylmethyl)-N-(1-methylpiperidin-4-yl)-N′-(4-(2-methylpropyloxy)phenylmethyl)carbamide orally by table or capsule (34 mg of compounds). Tablets may contain 20 mg of pimavanserin tartrate, which is equivalent to 17 mg of pimavanserin free base, or 11.8 mg of pimavanserin tartrate, which is equivalent to 10 mg of pimavanserin free base. In the above formulations, pimavanserin is in crystalline and/or amorphous form.

Pimavanserin (i.e., N-(4-fluorophenylmethyl)-N-(1-methylpiperidin-4-yl)-N′-(4-(2-methylpropyloxy)phenylmethyl)carbamide) or a pharmaceutically acceptable salt thereof (e.g., tartrate salt) may be administered in the form of a capsule, e.g. a capsule may contain 40 mg of pimavanserin tartrate, which is equivalent to 34 mg of pimavanserin free base, or 20 mg of pimavanserin tartrate, which is equivalent to 17 mg of pimavanserin free base, or 11.8 mg of pimavanserin tartrate, which is equivalent to 10 mg of pimavanserin free base.

In some embodiments, the drug product is formulated with standard pharmaceutical excipients at a 17 mg strength (20 mg of pimavanserin tartrate) in immediate-release tablets for once-daily oral administration.

In some embodiments, the drug product is formulated with standard pharmaceutical excipients at a 10 mg strength (11.8 mg of pimavanserin tartrate) in immediate-release tablets for once-daily or twice-daily oral administration.

Pimavanserin once daily a 10 mg tablet can, for instance, be administered to the subject when a strong CYP3A4 inhibitor (e.g., ketoconazole) is also being administered to the subject.

In some embodiments, the dose for the indication of adjunctive treatment of MDD indication is 34 mg pimavanserin taken orally as two 17 mg tablets once daily.

In some embodiments, the dose and pharmaceutical composition of pimavanserin are those disclosed in International Application No. PCT/US2018/048096, which is incorporated herein for all purposes. Pimavaserin can be in a crystalline form. Such forms have been described as Forms A, B, C, D, etc., e.g. in WO 2006/037043, WO 2007/133802 and WO 2008/144326.

In some embodiments, the pharmaceutical composition comprises granulated pimavanserin tartrate, Form C which may include a small percentage of Form A, including a pharmaceutically acceptable diluent, binder or excipient, or combination thereof.

In some embodiments, the pharmaceutical compositions are provided as a two-piece hard shell capsules made of gelatin (fish, mammalian, or vegetable sourced) or other combinations. The two-piece hard shell capsules may contain the pimavanserin granules with a filler/diluent and/or lubricants. In some embodiments, the capsules are size 3 or 4 capsules. In some embodiments, the capsules are size 4 capsules. In some embodiment, the capsules are two-piece capsules of gelatin or hydroxypropyl methylcellulose (HPMC). Some commercial examples are VCAPS®, VCAPS® PLUS, CONI-SNAP® capsules marketed by Capsugel.

In some embodiments, the doses referred to herein refers to pimavanserin free base. In some embodiments, the doses referred to herein refers to pimavanserin tartrate Form C (e.g., 40 mg of pimavanserin tartrate, equivalent to 34 mg pimavanserin free base). In some embodiments, the doses refer to pimavanserin tartrate Form C encapsulated in capsules of size 3 or 4, such as capsules of size 4.

Provided are also embodiments wherein pimavanserin (granulated), microcrystalline cellulose, for example Avicel P H302 or an equivalent microcrystalline cellulose, and/or magnesium stearate, for example vegetable grade are encapsulated in a capsule of size 4, for example a two-piece capsule.

One embodiment of the compositions described herein includes pimavanserin tartrate granulation without binder, dried, and thereafter blended with less than 60% w/w microcrystalline cellulose such as Avicel P H302 or equivalent microcrystalline cellulose, and about 1% w/w magnesium stearate.

In some embodiments the compositions described herein comprise granulated pimavanserin and microcrystalline cellulose is at least 20% w/w microcrystalline cellulose, such as 30% w/w microcrystalline cellulose, such as 40% w/w microcrystalline cellulose, such as 50% w/w microcrystalline cellulose, such as 50-89% w/w microcrystalline cellulose, such as 20-94% w/w, such as 50-94% w/w, such as 57-94% w/w, such as 57-89% w/w microcrystalline cellulose, such as 57-79% w/w microcrystalline cellulose, or 57-60% w/w microcrystalline cellulose, or 57-59.5% w/w microcrystalline cellulose, or 58.5-59.5% w/w microcrystalline cellulose, or 59% w/w microcrystalline cellulose.

In some embodiments the compositions described herein comprise granulated pimavanserin and microcrystalline cellulose and magnesium stearate, such as 0.1-3% w/w, such as 0.5-2% w/w magnesium stearate, or 0.5-1.5% w/w magnesium stearate, or 1% w/w magnesium stearate.

In some embodiments the compositions described herein comprise granulated pimavanserin (5, 10, 20 or 34 mg) and microcrystalline cellulose is at least 20% w/w microcrystalline cellulose, such as 30% w/w microcrystalline cellulose, such as 40% w/w microcrystalline cellulose, such as 50% w/w microcrystalline cellulose, such as 50-89% w/w microcrystalline cellulose, such as 20-94% w/w, such as 50-94% w/w, such as 57-94% w/w, such as 57-89% w/w microcrystalline cellulose, such as 57-79% w/w microcrystalline cellulose, or 57-60% w/w microcrystalline cellulose, or 57-59.5% w/w microcrystalline cellulose, or 58.5-59.5% w/w microcrystalline cellulose, or 59% w/w microcrystalline cellulose and magnesium stearate, such as 0.1-3% w/w, such as 0.5-2% w/w magnesium stearate, or 0.5-1.5% w/w magnesium stearate, or 1% w/w magnesium stearate.

The compositions disclosed herein comprise pimavanserin and additional compatible excipients, e.g. sugars, sucrose, mannitol, sorbitol, polysaccharides (e.g. from corn, wheat, rice, potato), as well as pregelatinized or partially pregelatinized starches (e.g. STARCH 1500®), cellulose preparations such as microcrystalline cellulose (MCC) (e.g. AVICEL® PH 302, AVICEL® PH 102, VIVAPUR® 302, VIVAPUR® 102, EMCOCEL® HD 90), silicified microcrystalline cellulose (e.g. PROSOLV® 50, PROSOLV® 90, PROSOLV® HD90), lactose cellulose blends (e.g. CELLATOSE® 80, CELLATOSE® 90, PROSOLV® EASYtab SP), hydroxypropylmethyl cellulose, hydroxymethyl cellulose, polyvinylpyrrolidone, lubricants such as magnesium stearate, sodium stearyl fumarate, colloidal silicon dioxide, and talc.

Provided are also embodiments wherein 34 mg pimavanserin (granulated) (equivalent to 40 mg pimavanserin tartrate), microcrystalline cellulose, such as microcrystalline cellulose having a particle size distribution (D90) of 180-340 μm, for example Avicel P H302 or an equivalent microcrystalline cellulose, and/or magnesium stearate, for example vegetable grade are encapsulated in a capsule of size 4, for example a two-piece capsule.

Provided are also embodiments wherein 10 or 20 mg pimavanserin (granulated), microcrystalline cellulose, for example Avicel PH302 or an equivalent microcrystalline cellulose, and/or magnesium stearate, for example vegetable grade are encapsulated in a capsule of size 4, for example a two-piece capsule.

Provided are also embodiments wherein 34 mg pimavanserin (granulated), 59 mg microcrystalline cellulose, for example Avicel PH302 or an equivalent microcrystalline cellulose, and/or 1 mg magnesium stearate, for example vegetable grade are encapsulated in a capsule of size 4, for example a two-piece capsule. No other excipients were added.

Also provided is a pharmaceutical composition, comprising a capsule of pimavanserin and one or more pharmaceutically acceptable excipient(s) as provided herein, wherein the composition is formulated such that at least 80% of pimavanserin is released in 30 minutes upon administration to a subject.

Also provided is a pharmaceutical composition, comprising a capsule of pimavanserin and one or more pharmaceutically acceptable excipient(s) as provided herein, wherein the composition is formulated such that at least 80% of the pimavanserin is released from the composition within 30 minutes upon in vitro dissolution testing according to USP<711> (apparatus 1 (basket apparatus)).

In some embodiments, a pharmaceutically acceptable salt of pimavanserin is administered to the patient. In some specific embodiments, a tartrate salt of pimavanserin is administered to the patient.

In some embodiments, the pharmaceutically acceptable salt of pimavanserin comprises an anion selected from the group consisting of phosphate, sulphate, nitrate, diphosphate, besylate, bicarbonate, carbonate, clavulanate, edisylate, isothionate, borate, halide including e.g., chloride and bromide, nitrate, acetate, succinate, lactate, lactobionate, laurate, mandelate, malate, citrate, fumarate, maleate, oleate, oxalate, ascorbate, nicotinate, benzoate, mesylate, salicylate, stearate, tannate, tosylate, valerate, methanesulfonate, ethanesulfonate, benzenesulfonate, p-toluensulfonate, 2-ethane disulfonate, and naphthalenesulfonate.

The exact route of administration, dose, or frequency of administration would be readily determined by those skilled in the art and can be dependent on the age, weight, general physical condition, or other clinical symptoms specific to the patient to be treated.

In some embodiments, the tartrate salt of pimavanserin is administered daily. In some embodiments, the tartrate salt of pimavanserin is administered once daily. In some embodiments, the tartrate salt of pimavanserin is formulated for oral administration as a unit dose.

In an embodiment, pimavanserin is administered orally.

In an embodiment, pimavanserin is orally administered in a daily dose from about 0.5 mg to about 90 mg, or from about 8 mg to about 42 mg, or about 10 mg to about 60 mg.

In an embodiment, pimavanserin is orally administered in a daily dose from about 0.5 mg to about 120 mg, or from about 8 mg to about 42 mg, or about 10 mg to about 60 mg.

In certain embodiments, the above ranges are for pimavanserin free base. In certain embodiments, the above ranges are for a pharmaceutically acceptable salt, e.g., a tartrate salt of pimavanserin or any of the salts listed above.

In another embodiment, pimavanserin tartrate is orally administered in a daily dose of about 40 mg. In some embodiments, the daily dose is 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 80 mg, 88 mg or 102 mg. In an embodiment the daily dose of pimavanserin tartrate is administered once, twice or three times per day, for example a 40 mg dose of pimavanserin tartrate is administered once a day, or 20 mg pimavanserin tartrate is administered twice a day.

In another embodiment, pimavanserin is orally administered in a daily dose of about 34 mg. In some embodiments, the daily dose is 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 68 mg, 80 mg, 88 mg or 102 mg. In an embodiment the daily dose of pimavanserin is administered once, twice or three times per day, for example a 34 mg dose of pimavanserin is administered once a day, a 20 mg dose of pimavanserin once or twice a day, or 17 mg pimavanserin is administered twice a day, or a 10 mg dose pimavanserin is administered twice a day.

### Antidepressants

In some embodiments, the SSRI is selected from the group consisting of citalopram, escitalopram (LEXAPRO), fluoxetine (PROZAC), fluvoxamine, paroxetine (PAXIL), sertraline, vilazodone and vortioxetine.

In some embodiments, the SNRI is selected from the group consisting of desvenlafaxine, duloxetine, levomilnacipran, sibutramine, tramadol, milnacipran and venlafaxine.

In certain embodiments in the methods provided herein, wherein the human subject is on antidepressant therapy, the antidepressant given to the subject can, for example, be doxepin, clomipramine, amoxapine, amitriptyline, maprotiline, desipramine, nortriptyline, doxepin, trimipramine, imipramine, protriptyline, pipofezine or noxiptiline.

In some embodiments the antidepressant is a tricyclic antidepressant. Tricyclic antidepressants are known in the art. In certain embodiments the antidepressant is an antipsychotic.

In certain embodiments of the methods provided herein wherein the subject is on antidepressant therapy, the antidepressant can be bupropion or nefazodone.

### Sexual Dysfunction

Sexual dysfunction can, for example, be determined according any method known in the art.

Assessment of sexual dysfunctioning can, for example, be a subject's self-assessment or self-reporting on his or her sexual function.

In certain embodiments, a survey or questionnaire is conducted to determine sexual dysfunction in a subject. Exemplary survey or questionnaire include, for example, Changes in Sexual Functioning Questionnaire (CSFQ-F-C) and Arizona Sexual Experiences Scale (ASEX).

In certain embodiments, sexual dysfunction is assessed using the Massachusetts General Hospital Sexual Functioning Index (“MGH-SFI”), described, for instance, in Labbate and Lare, 2001, Psychother Psychosom. 70(4):221-225.

In the methods provided herein for treating sexual dysfunction, the sexual dysfunction can, for example, be loss of interest in sex, loss of sexual arousal, diminished ability or inability to achieve orgasm, loss of ability to maintain an erection in male subjects, loss of sexual satisfaction, reduced frequency of sexual activity, or reduced satisfaction from sexual activity.

Those skilled in the art will recognize that treating sexual dysfunction can constitute improving sexual function. For instance, a sexual dysfunction such as, for example, loss of interest sex when treated can be an improvement in sexual function (increased interest in sex); loss of sexual arousal (dysfunction) when treated can be an improvement in sexual function by gain of sexual arousal, and so forth.

Also provided herein are methods for augmenting relief of depression comprising administering to a human subject on antidepressant therapy a selective serotonin 5-HT_{2A} inverse agonist or antagonist, e.g., pimavanserin or a pharmaceutically acceptable salt thereof.

### EXAMPLES

### Example 1: Study of Adjunctive Pimavanserin in Patients with Major Depressive Disorder and an Inadequate Response to Therapy

A multicenter, randomized, double-blind, placebo-controlled study of the efficacy and safety of pimavanserin in patients with DSM-5—defined MDD and an inadequate response to an SSRI or SNRI was conducted. Key endpoints were change from baseline to the end of each stage for the HAMD-17 total score and SDS score.

The study consisted of an 8- to 21-day screening period, a 10-week double-blind treatment period, and a safety follow up period of up to 30 days. Screening assessments consisted of the SAFER Interview (Desseilles et al, Harv Rev Psychiatry. 2013; 21(5):269-74), which included the MADRS, CGI-S, and MGH ATRQ.

Patients were randomly assigned via an interactive voice response system to pimavanserin 34 mg (provided as two 17 mg NUPLAZID® tablets) or placebo (provided by two tablets) once daily by mouth or placebo. All patients continued on their SSRI or SNRI at a stable dose for the duration of the study.

The study utilized a 2-stage Sequential Parallel-Comparison Design (SPCD) (Fava et al, Psychother Psychosom. 2003; 72:115-127) whereby, following screening, eligible patients were randomized in Stage 1 in a 3:1 ratio to placebo or pimavanserin added to their current SSRI or SNRI therapy for 5 weeks. At the end of 5 weeks, placebo non-responders (HAMD-17 total score >14 and <50% reduction in score from baseline) were re-randomized to placebo or pimavanserin (1:1 ratio) added to current therapy for an additional 5 weeks. All patients assigned to pimavanserin in Stage 1 continued treatment with pimavanserin in Stage 2, whereas responders to placebo in Stage 1 remained on placebo in Stage 2.

### Subjects Selection

Male or female patients ≥18 years of age were eligible if they had a primary diagnosis of MDD and major depressive episode (MDE) as defined by the DSM-5 and confirmed by the Structured Clinical Interview for DSM-5, Clinical Trials Version (SCID-5-CT). Eligible patients had a history of MDD for ≥1 year prior to screening, a MADRS total score ≥20, and a CGI-S score ≥4 (moderately ill or worse) at both screening and baseline. Eligible patients also had a history of an inadequate response to 1 or 2 antidepressant treatments during the current depression episode. Inadequate treatment response was determined through the administration of the MGH ATRQ administered during the SAFER interview. Eligible patients received treatment for their current episode with exactly one of the following drugs at approved doses: citalopram, desvenlafaxine, duloxetine, escitalopram, fluoxetine, paroxetine, sertraline, venlafaxine, or venlafaxine XR.

### Study Assessments

Clinic visits occurred weekly from Weeks 1 through 10. The MADRS was administered at screening and baseline. The HAMD-17, SDS, CGI, and KSS were administered at baseline and weekly during the study. Other endpoints such as the SF-12 were administered at baseline and Weeks 5 and 10. Treatment response was defined as a reduction of 50% from baseline for the HAMD-17 total score, and remission was defined as a HAMD-17 total score

### Results

Patients with MDD

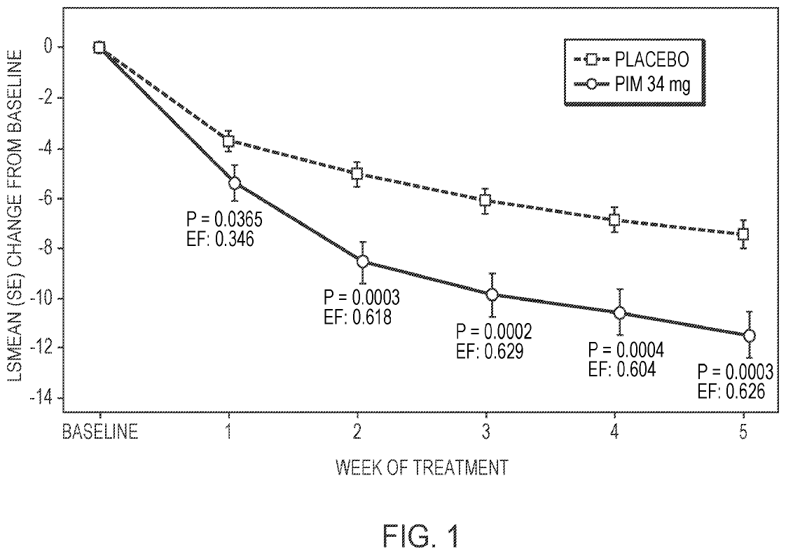

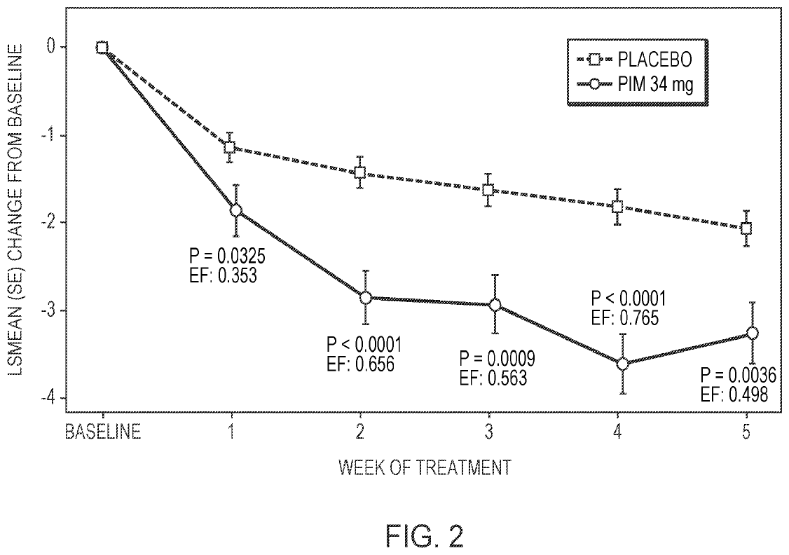

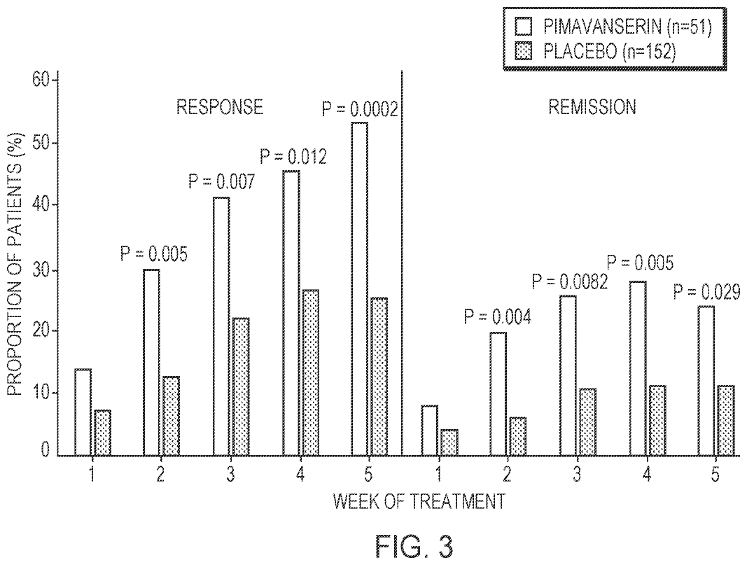

A significantly greater improvement was observed with pimavanserin than placebo for the HAMD-17 total (LSmean difference [standard error]-1.7 (0.85), p=0.039) and the SDS (LSmean difference −0.8 (0.29), p=0.004). At Week 5 of Stage 1, LSmean (standard error) change from baseline for the HAMD-17 was −11.5 (0.94) for pimavanserin and −7.5 (0.55) for placebo (LSmean difference −4.0 [1.09], p=0.0003; effect size 0.626) (FIG. 1), and for SDS, LSmean change was −3.3 (0.35) for pimavanserin and −2.1 (0.20) for placebo (LSmean difference −1.2 [0.40] p=0.0036, effect size 0.498) (FIG. 2). The NNT for response and remission in Stage 1 was 3.6 and 8.1, respectively. During Stage 1, response and remission rates were significantly (p<0.05) greater with pimavanserin vs. placebo from Week 2 through Week 5 (FIG. 3). In Stage 2, the difference between pimavanserin and placebo was not significant for the HAMD-17 (delta=0.5; p=0.6940; Cohen's d=−0.107).

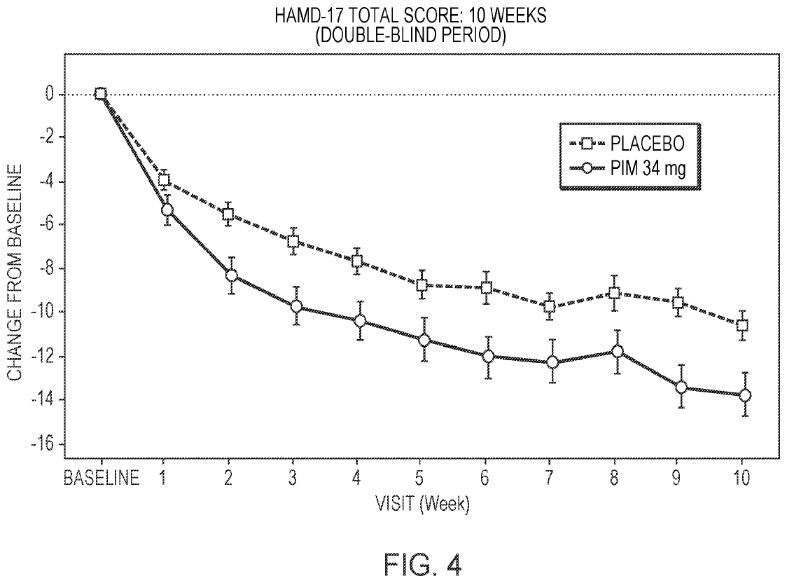

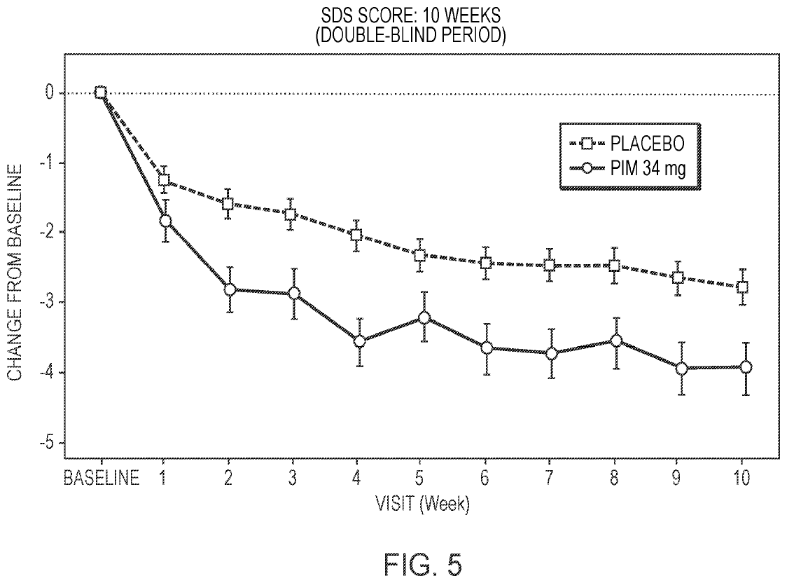

In a prespecified analysis of patients receiving continuous treatment with pimavanserin or placebo for 10 weeks in Stages 1 and 2, the treatment difference in LSmean change from baseline to Week 10 was significant for both the HAMD-17 (p=0.0076, effect size 0.497) and the SDS (p=0.0094, effect size 0.469) in favor of pimavanserin (FIG. 4 and FIG. 5).

Patients with MDD and Anxious Distress

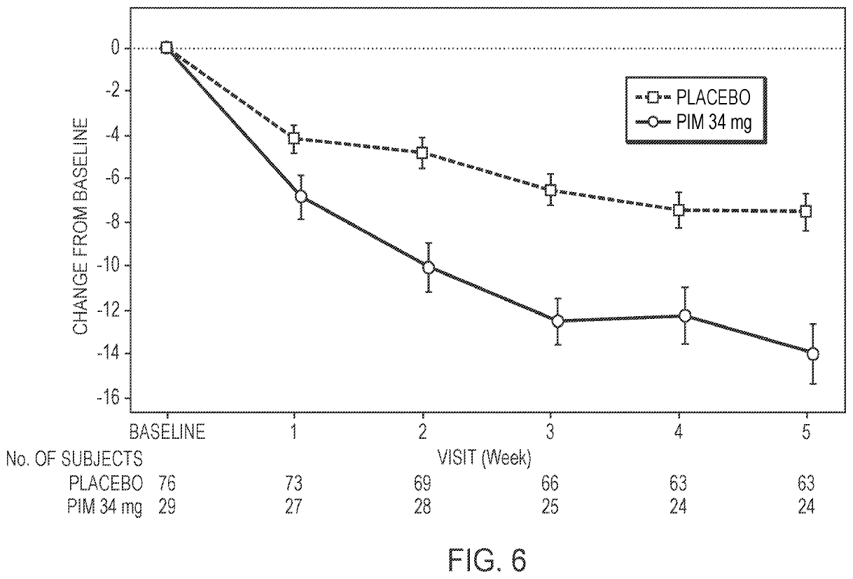

Robust improvement on HAMD-17 total scores among subjects with anxious distress at baseline (subjects with baseline HAMD-17 anxiety/somatization subscale score ≥7) was observed with pimavanserin in Stage 1 (p=0.0001, effect size 0.940) (Table 1, FIG. 6). A higher HAMD-17 anxiety/somatization subscale score indicates more severe depression with anxiety symptoms. A positive effect size favors pimavanserin.

TABLE 1
HAMD-17 total score analysis for subgroup with
baseline anxiety/somatization subscale score ≥7

| | Stage 1 | | Stage 2 | |
|---|---|---|---|---|
| | Placebo | PIM | Placebo | PIM |
| | (n = 76) | (n = 29) | (n = 18) | (n = 19) |
| Baseline | | | | |
| n | 76 | 29 | 18 | 19 |
| Mean (SE) | 24.4 (0.44) | 25.6 (0.65) | 22.1 (1.04) | 21.4 (1.13) |
| Change from Baseline | | | | |
| to Week 5 | | | | |
| n | 63 | 24 | 15 | 19 |
| LSM (SE) | −7.5 (0.85) | −14.1 (1.39) | −4.3 (1.34) | −3.4 (1.25) |
| 95% CI of LSM | (−9.2, −5.8) | (−16.9, −11.4) | (−7.0, −1.6) | (−5.9, −0.8) |
| Diff in LSM (SE) | | −6.6 (1.64) | | 0.9 (1.84) |
| 95% CI of Diff | | (−9.8, −3.3) | | (−2.8, 4.7) |
| p-value | | 0.0001 | | 0.6129 |
| Effect Size (Cohen's d) | | 0.940 | | −0.173 |
| Overall Treatment Comparison | | | | |
| Weighted Diff in LSM (SE) | | | | −2.8 (1.23) |
| 95% CI of Weighted Diff | | | | (−5.2, −0.4) |
| in LSM | | | | |
| p-value | | | | 0.0219 |

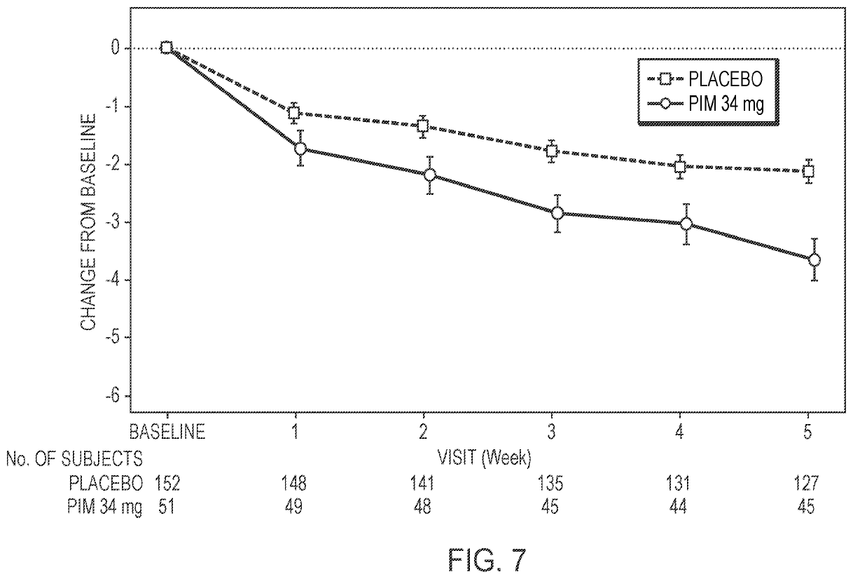

Improvement on HAMD-17 “anxiety somatization” item was observed with pimavanserin in Stage 1 (p=0.0003, effect size 0.634) and Overall comparisons (p=0.0166) (Table 2, FIG. 7). A higher HAMD-17 anxiety/somatization subscale score indicates more severe depression with anxiety symptoms. A positive effect size favors pimavanserin.

TABLE 2
HAMD-17 anxiety/somatization subscale score

| | Stage 1 | | Stage 2 | |
|---|---|---|---|---|
| | Placebo | PIM | Placebo | PIM |
| | (n = 152) | (n = 51) | (n = 29) | (n = 29) |
| Baseline | | | | |
| n | 152 | 51 | 29 | 29 |
| Mean (SE) | 6.6 (0.18) | 6.9 (0.36) | 6.0 (0.43) | 6.5 (0.49) |
| Change from Baseline | | | | |
| to Week 5 | | | | |
| n | 127 | 45 | 24 | 29 |
| LSM (SE) | −2.1 (0.21) | −3.7 (0.36) | −0.6 (0.35) | −0.6 (0.33) |
| 95% CI of LSM | (−2.5, −1.7) | (−4.4, −3.0) | (−1.3, 0.1) | (−1.3, 0.0) |
| Diff in LSM (SE) | | −1.5 (0.41) | | 0.0 (0.48) |
| 95% CI of Diff | | (−2.4, −0.7) | | (−1.0, 1.0) |
| p-value | | 0.0003 | | 0.9799 |
| Effect Size (Cohen's d) | | 0.634 | | −0.007 |
| Overall Treatment Comparison | | | | |
| Weighted Diff in LSM (SE) | | | | −0.8 (0.32) |
| 95% CI of Weighted Diff | | | | (−1.4, −0.1) |
| in LSM | | | | |
| p-value | | | | 0.0166 |

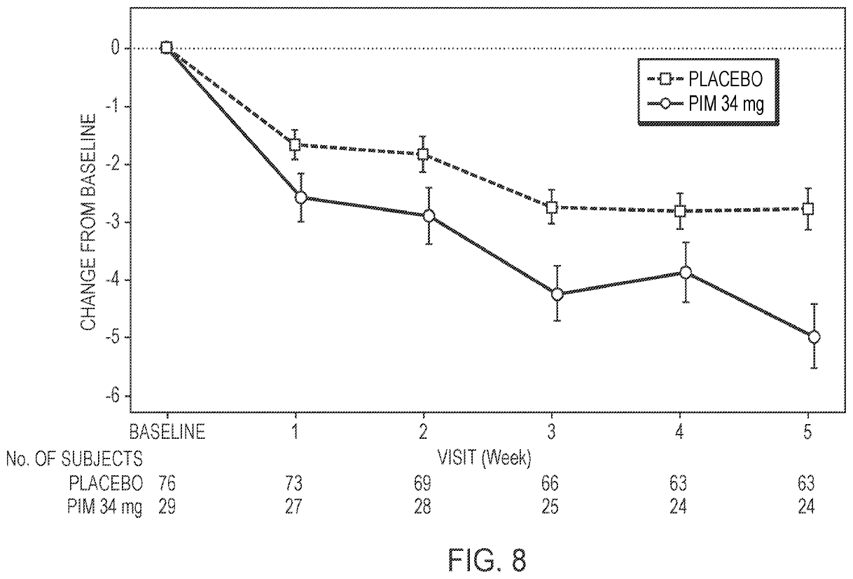

Robust improvement on “anxiety somatization” symptoms among the subgroup of subjects with higher anxious distress scores at baseline (subjects with baseline HAMD-17 anxiety/somatization subscale score ≥7) was observed with pimavanserin in Stage 1 (p=0.0013, effect size 0.781) (Table 3, FIG. 8).

TABLE 3
HAMD-17 anxiety/somatization subscale score for subgroup
with baseline anxiety/somatization subscale score ≥7

| | Stage 1 | | Stage 2 | |
|---|---|---|---|---|
| | Placebo | PIM | Placebo | PIM |
| | (n = 76) | (n = 29) | (n = 18) | (n = 19) |
| Baseline | | | | |
| n | 76 | 29 | 18 | 19 |
| Mean (SE) | 8.5 (0.15) | 8.8 (0.28) | 7.1 (0.54) | 7.5 (0.59) |
| Change from Baseline | | | | |
| to Week 5 | | | | |
| n | 63 | 24 | 15 | 19 |
| LSM (SE) | −2.8 (0.35) | −5.0 (0.56) | −1.5 (0.49) | −1.3 (0.44) |
| 95% CI of LSM | (−3.5, −2.1) | (−6.1, −3.8) | (−2.5, −0.5) | (−2.3, −0.4) |
| Diff in LSM (SE) | | −2.2 (0.66) | | 0.1 (0.66) |
| 95% CI of Diff | | (−3.5, −0.9) | | (−1.2, 1.5) |
| p-value | | 0.0013 | | 0.8465 |
| Effect Size (Cohen's d) | | 0.781 | | −0.067 |
| Overall Treatment Comparison | | | | |
| Weighted Diff in LSM (SE) | | | | −1.0 (0.47) |
| 95% CI of Weighted Diff | | | | (−2.0, −0.1) |
| in LSM | | | | |
| p-value | | | | 0.0271 |

### Early Improvement

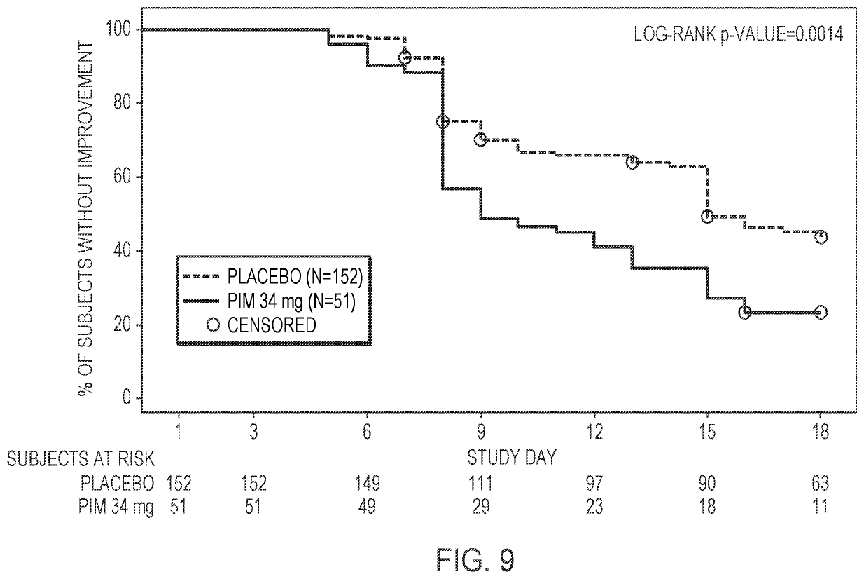

As shown in FIG. 9, early improvement at week 1 was observed with pimavanserin compared to placebo. Early improvement is defined as a reduction from baseline in HAMD-17 total score of 20% or more.

### HAMD-17 Individual Items

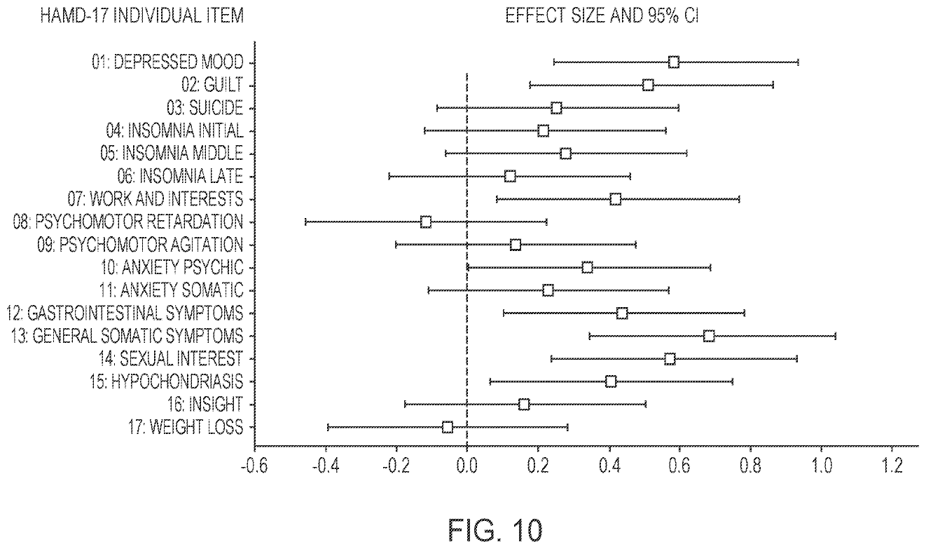

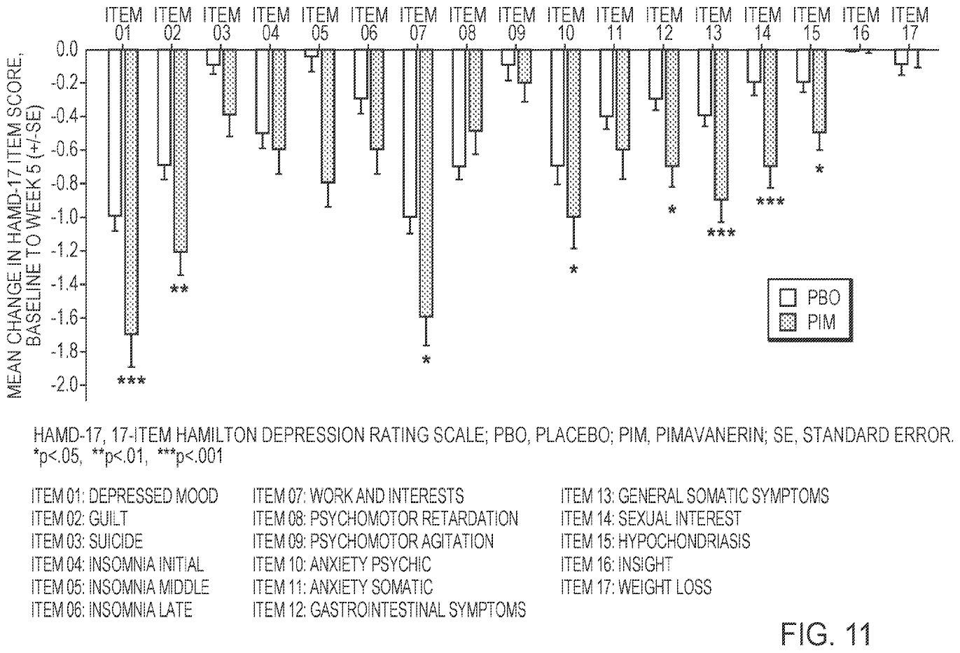

FIG. 10 shows effect size and 95% CI for HAMD-17 items at Stage 1 Week 5. FIG. 11 shows the mean change in HAMD-17 individual item scores from baseline to Week 5 with pimavanserin or placebo.

Patients with Melancholic Symptoms with MDD

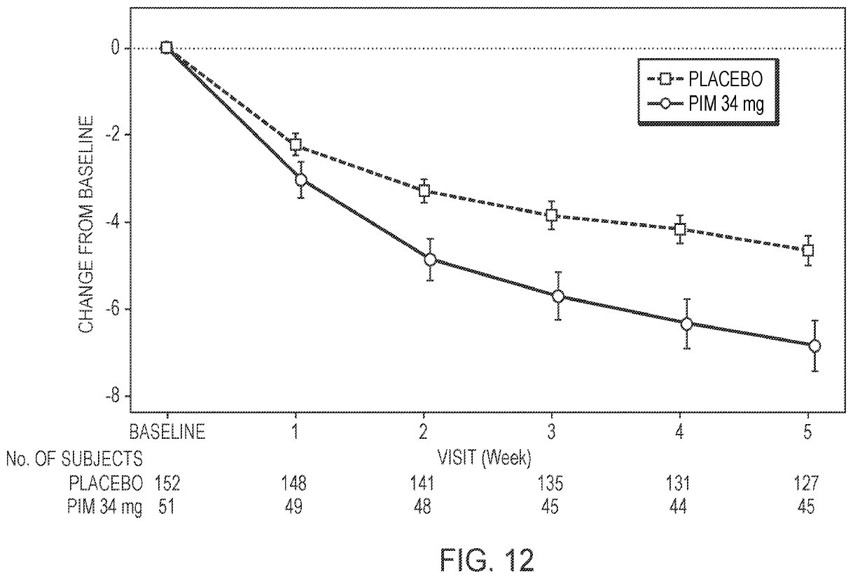

Improvement on HAMD-17 melancholic symptom subscale score (depressed mood, guilt feelings, work and interests, psychomotor retardation, psychic anxiety, and general somatic symptoms items of HAMD-17 (i.e., HAMD-6 score)) was observed with pimavanserin in Stage 1 (p=0.0012, effect size 0.558) (Table 4, FIG. 12). A higher HAMD-17 melancholic symptom subscale score indicates more severe depression with anxiety symptoms. The abbreviations are: CI=confidence interval; LSM=least squares mean; MMRM=mixed-effects model for repeated measures; OC=observed cases; SD=standard deviation; SE=standard error. In Stage 1, Baseline=Study Week 0, Weeks 1-5=Study Weeks 1-5; and in Stage 2, Baseline=Study Week 5, Weeks 1-5=Study Weeks 6-10. LSM is from the stage-specific MMRM analysis with the change from baseline as the outcome; treatment group, visit, treatment-by-visit interaction, baseline melancholic symptom subscale score, and baseline melancholic symptom subscale score-by-visit interaction as the factors. An unstructured covariance matrix is used to model the within-subject errors. The denominator degrees of freedom are estimated using the Kenward-Roger approximation. 2-Sided p-value is for treatment difference from the stage-specific MMRM analysis.

TABLE 4
HAMD-17 Melancholic Symptom Subscale Score (based on HAMD-6)

| | Stage 1 | | Stage 2 | |
|---|---|---|---|---|
| | Placebo | Pimavanserin | Placebo | Pimavanserin |
| Visit | (N = 152) | (N = 51) | (N = 29) | (N = 29) |
| Baseline | | | | |
| n | 152 | 51 | 29 | 29 |
| Mean (SE) | 12.6 (0.15) | 12.5 (0.29) | 11.4 (0.47) | 11.0 (0.44) |
| SD | 1.86 | 2.04 | 2.56 | 2.35 |
| Median | 13.0 | 12.0 | 12.0 | 11.0 |
| Min, Max | 7, 19 | 8, 16 | 6, 17 | 7, 16 |
| Change from Baseline | | | | |
| to Week 5 | | | | |
| n | 127 | 45 | 24 | 29 |
| Mean (SE) | −4.5 (0.35) | −6.8 (0.62) | −1.8 (0.62) | −1.0 (0.46) |
| SD | 3.95 | 4.18 | 3.03 | 2.49 |
| Median | −4.0 | −8.0 | −1.5 | −1.0 |
| Min, Max | −15, 3 | −16, 1 | −10, 3 | −7, 3 |
| MMRM LSM (SE) | −4.7 (0.34) | −6.9 (0.58) | −1.8 (0.55) | −1.0 (0.52) |
| 95% CI of LSM | (−5.3, −4.0) | (−8.0, −5.7) | (−2.9, −0.7) | (−2.0, 0.1) |
| Diff (PIM 34 mg-Placebo) | | −2.2 (0.67) | | 0.8 (0.76) |
| in MMRM LSM (SE) | | | | |
| 95% CI of Diff | | (−3.5, −0.9) | | (−0.7, 2.4) |
| MMRM p-value | | 0.0012 | | 0.2706 |
| Effect Size (Cohen's d) | | 0.558 | | −0.304 |
| Overall Treatment Comparison | | | | |
| at Week 5 (Linear Combination | | | | |
| Test) | | | | |
| Weighted Diff in MMRM LSM (SE) | | | | −0.7 (0.51) |
| 95% CI of Weighted Diff | | | | (−1.7, 0.3) |
| p-value | | | | 0.1786 |

### Functional Remission Rate

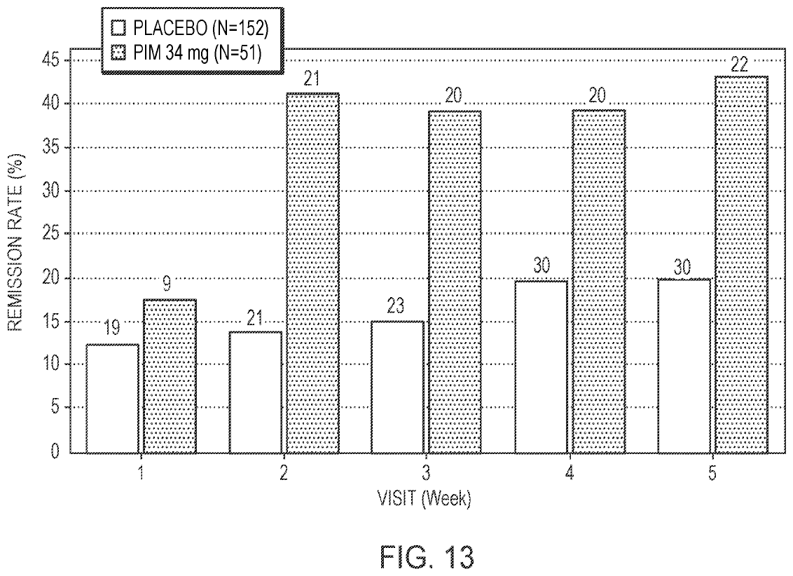

Significant increase in functional remission was observed with pimavanserin compared to placebo in Stage 1 (Table 5, FIG. 13). In Stage 1, weeks 1-5 is the study weeks 1-5 and in Stage 2, weeks 1-5 is the study weeks 6-10. 95% CI refers to Newcombe 95% confidence interval. The p-value is from stage-specific Pearson's Chi-square test. Functional remission is defined as SDS total score <6.

TABLE 5
Functional Remission Rate by Visit

| | | Stage 1 | | Stage 2 | |
|---|---|---|---|---|---|
| | | Placebo | Pimavanserin | Placebo | Pimavanserin |
| Visit | Category | (N = 152) | (N = 51) | (N = 29) | (N = 29) |
| Week 1 | Remission | | | | |
| | Remitter, n(%) | 19 (12.5) | 9 (17.6) | 3 (10.3) | 2 (6.9) |
| | Diff (PIM 34 mg-Placebo) | | 5.1 | | −3.4 |
| | in Remission Rate (%) | | | | |
| | 95% CI[2] | | (−5.0, 18.5) | | (−20.2, 13.1) |
| | p-value[3] | | 0.3563 | | 0.6399 |
| Week 2 | Remission | | | | |
| | Remitter, n(%) | 21 (13.8) | 21 (41.2) | 3 (10.3) | 3 (10.3) |
| | Diff (PIM 34 mg-Placebo) | | 27.4 | | 0.0 |
| | in Remission Rate (%) | | | | |
| | 95% CI[2] | | (13.4, 41.8) | | (−17.4, 17.4) |
| | p-value[3] | | <.0001 | | 1.0000 |
| Week 3 | Remission | | | | |
| | Remitter, n(%) | 23 (15.1) | 20 (39.2) | 3 (10.3) | 3 (10.3) |
| | Diff (PIM 34 mg-Placebo) | | 24.1 | | 0.0 |
| | in Remission Rate (%) | | | | |
| | 95% CI[2] | | (10.2, 38.6) | | (−17.4, 17.4) |
| | p-value[3] | | 0.0003 | | 1.0000 |
| Week 4 | Remission | | | | |
| | Remitter, n(%) | 30 (19.7) | 20 (39.2) | 2 (6.9) | 4 (13.8) |
| | Diff (PIM 34 mg-Placebo) | | 19.5 | | 6.9 |
| | in Remission Rate (%) | | | | |
| | 95% CI[2] | | (5.4, 34.3) | | (−10.3, 24.4) |
| | p-value[3] | | 0.0052 | | 0.3885 |
| Week 5 | Remission | | | | |
| | Remitter, n(%) | 30 (19.7) | 22 (43.1) | 2 (6.9) | 6 (20.7) |
| | Diff (PIM 34 mg-Placebo) | | 23.4 | | 13.8 |
| | in Remission Rate (%) | | | | |
| | 95% CI[2] | | (8.9, 38.1) | | (−4.8, 32.2) |
| | p-value[3] | | 0.0009 | | 0.1277 |
| Overall Treatment | Remission | | | | |
| Comparison at | Weighted Treatment Diff | | | | 18.6 |
| Week 5 (Linear | in Remission Rate (%) | | | | |
| Combination Test) | 95% CI of Weighted Diff | | | | (7.1, 30.1) |
| | p-value | | | | 0.0015 |

### SDS Individual Items

“work/school,” “social life,” “family life/home responsibilities”

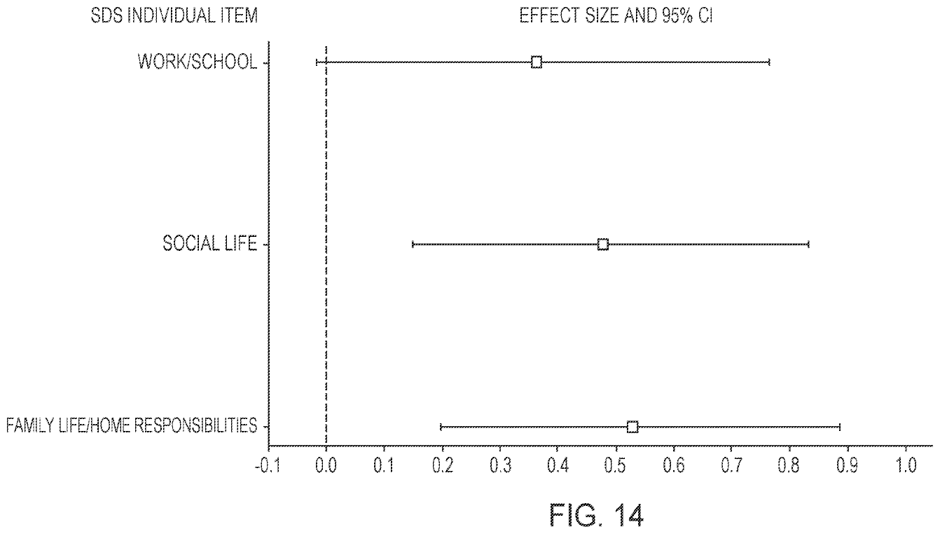

FIG. 14 shows effect size and 95% CI for SDS individual items (“work/school,” “social life,” “family life/home responsibilities”) at Stage 1 Week 5. A positive effect size favors pimavanserin over placebo. As shown in the figure, a positive effect size was observed for all the 3 individual items.

As shown in FIG. 14, pimavanserin enhances the functional improvement on the three individual items by having a positive treatment effect size (positive values on the x-axis; on the right side of the value of 0 line (dotted line in the middle) as well as a positive 95% confidence interval (CI) of Effect Size (the lower limit of the interval is positive and on the right of the dotted line).

### “Days Unproductive”

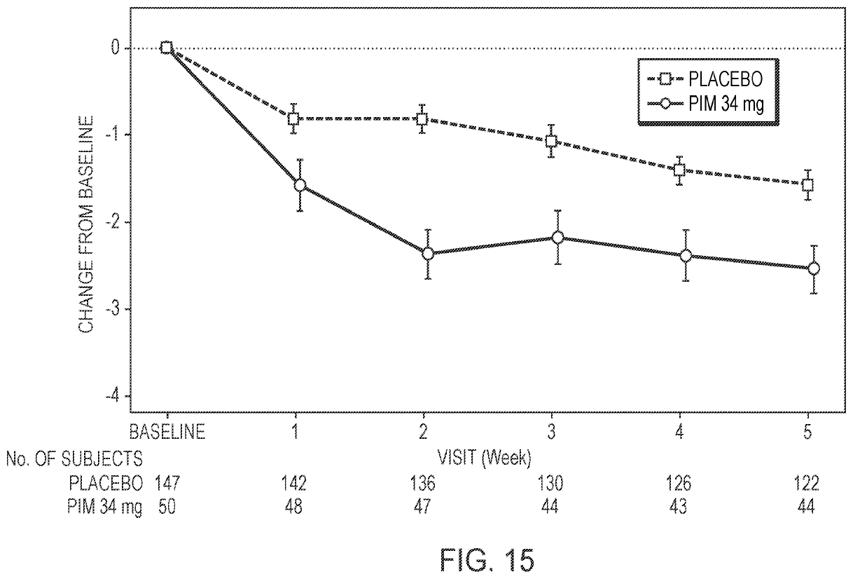

Significant reduction in unproductive days in school or work was observed with pimavanserin compared to placebo in Stage 1 (Table 6, FIG. 15).

Days Unproductive is an item of SDS and a patient's response to: “on how many days in the last week did you feel so impaired by your symptoms, that even though you went to school or work, your productivity was reduced?” As shown in Table 6, Week 5 Difference (Pimavanserin-Placebo) LSM (SE) is −0.970 (0.3222) unproductive days per week for pimavanserin compared to placebo (or about one less unproductive day/week).

CI=confidence interval; LSM=least squares mean; MMRM=mixed-effects model for repeated measures; OC=observed cases; SD=standard deviation; SE=standard error.

Higher SDS mean score indicates greater functional impairment. A positive effect size favors pimavanserin. In Stage 1, weeks 1-5 is the study weeks 1-5 and in Stage 2, weeks 1-5 is the study weeks 6-10. LSM from the stage-specific MMRM analysis with the change from baseline as the outcome; treatment group, visit, treatment-by-visit interaction, baseline SDS Day Unproductive, and baseline SDS Days Unproductive-by-visit interaction as the factors. 2-sided p-value for treatment difference from the stage-specific MMRM analysis.

TABLE 6
SDS Days Unproductive and Change from Baseline by Visit

| | Stage 1 | | Stage 2 | |
|---|---|---|---|---|
| | Placebo | Pimavanserin | Placebo | Pimavanserin |
| | (N = 152) | (N = 51) | (N = 29) | (N = 29) |
| Visit | | | | |
| Baseline | | | | |
| n | 147 | 50 | 29 | 29 |
| Mean (SE) | 3.605 (0.1833) | 3.220 (0.3160) | 2.724 (0.3643) | 2.931 (0.4180) |
| SD | 2.2224 | 2.2342 | 1.9621 | 2.2509 |
| Median | 4.000 | 3.000 | 2.000 | 2.000 |
| Min, Max | 0.00, 9.00 | 0.00, 7.00 | 0.00, 7.00 | 0.00, 7.00 |
| Visit[1] | | | | |
| Week 5 | | | | |
| n | 126 | 45 | 24 | 29 |
| Mean (SE) | 1.921 (0.1843) | 0.956 (0.2356) | 2.708 (0.4833) | 2.276 (0.4295) |
| SD | 2.0692 | 1.5805 | 2.3679 | 2.3130 |
| Median | 2.000 | 0.000 | 2.500 | 2.000 |
| Min, Max | 0.00, 7.00 | 0.00, 7.00 | 0.00, 7.00 | 0.00, 7.00 |
| Change from Baseline | | | | |
| to Week 5 | | | | |
| n | 122 | 44 | 24 | 29 |
| Mean (SE) | −1.631 (0.2400) | −2.364 (0.3253) | −0.333 (0.2530) | −0.655 (0.4689) |
| SD | 2.6511 | 2.1576 | 1.2394 | 2.5252 |
| Median | −1.000 | −2.000 | 0.000 | −1.000 |
| Min, Max | −7.00, 5.00 | −7.00, 1.00 | −3.00, 2.00 | −6.00, 5.00 |
| MMRM LSM (SE)[2] | −1.576 (0.1641) | −2.546 (0.2771) | −0.332 (0.3698) | −0.647 (0.3496) |
| 95% CI of LSM | (−1.900, −1.252) | (−3.093, −2.000) | (−1.072, 0.408) | (−1.348, 0.054) |
| Diff (PIM 34 mg-Placebo) | | −0.970 (0.3222) | | −0.315 (0.5089) |
| in MMRM LSM (SE) | | | | |
| 95% CI of Diff | | (−1.606, −0.334) | | (−1.335, 0.705) |
| MMRM p-value[3] | | 0.0030 | | 0.5385 |
| Effect Size (Cohen's d) | | 0.518 | | 0.167 |

### Tolerability

For the KSS measure of daytime sleepiness at Week 5 of Stage 1, LSmean difference from baseline for pimavanserin vs. placebo was −1.1 (0.30) (p=0.0003, effect size 0.627), indicating less somnolence among pimavanserin vs. placebo-treated subjects.

### Discussion

The results of this Phase 2 study showed statistically significant and clinically relevant and robust improvements in the HAMD-17 total score and SDS with pimavanserin as adjunctive therapy in patients with MDD who had an inadequate response to antidepressant therapy. Specifically, pimavanserin demonstrated an early and sustained separation from placebo at Week 1 during Stage 1 for both the HAMD-17 and the SDS. Importantly, the effect size was in the range of 0.6 and 0.5, respectively, for measures of depressive symptoms and function at Week 5 of Stage 1. In addition, significant improvements with pimavanserin were observed for a number of secondary endpoints including daytime sleepiness (KSS) and mental health-related quality of life (SF-12). Sleep disturbance is widely recognized by HCPs as an early indicator for worsening psychiatric symptoms and a trigger for urgency to treat.

Mean change from baseline to Week 5 for the HAMD-17 and SDS was significant, with substantial effect sizes of 0.626 and 0.498, respectively, and effect sizes were 0.497 and 0.469, respectively, for the HAMD-17 and SDS in the prespecified analysis of patients receiving continuous pimavanserin therapy for 10 weeks. In contrast, studies with AAPs as adjunctive therapy in patients with MDD report effect sizes of 0.27 to 0.43 symptoms (Mulder et al, Bipolar Disord. 2018; 20(suppl 2):17-24; Spielman et al. PLoS Med. 2013; 10(3):e1001403). Significant improvement in function on the SDS suggest potentially broader beneficial effects of pimavanserin as adjunctive treatment. Few studies with AAPs in MDD have included assessment of functional improvement.

Pimavanserin was well tolerated in this study. The AE profile was consistent with previous studies of pimavanserin for Parkinson's disease psychosis and Alzheimer's disease psychosis and discontinuations for AEs were lower with pimavanserin than with placebo (1.2% vs. 3.2%).

Importantly, pimavanserin was associated with low rates of daytime sleepiness, weight gain, metabolic laboratory tests, and sexual dysfunction. In contrast, use of AAPs may be limited by weight gain, metabolic disturbances (glucose intolerance, diabetes, lipid disorders, hyperprolactinemia), and daytime sleepiness (Mohamed et al. JAMA. 2017; 318(2):132-145; Solmi et al. Ther Clin Risk Manag. 2017; 13:757-777; Yoon et al. J Clin Psychopharmacol. 2017; 37(1):46-53; Mulder et al. Bipolar Disord. 2018; 20(suppl 2):17-24). In addition, conventional antidepressants are well known to cause sexual dysfunction in 50% or more of patients with MDD (Clayton et al. Expert Opin Drug Saf. 2014; 13(10):1361-1374).

### Example 2: Study of Safety and Efficacy of Pimavanserin Treatment in Adults with Parkinson's Disease and Depression

An 8-week, open-label, single arm study evaluating the safety and efficacy of 34 mg pimavanserin treatment of depression in adults with Parkinson's disease is conducted. Subjects can receive treatment as either monotherapy or adjunctively if inadequately controlled with SSRI/SNRI monotherapy. A primary endpoint of the study is change from baseline to Week 8 in the Hamilton Depression Scale 17-item (HAMD-17) total score. Secondary endpoints include Clinical Global Impression (CGI) scales (improvement and severity), Scale of Outcomes in PD-Sleep (SCOPA), and quality of life as measured by EuroQol-5 dimensions-5 levels (EQ-5D-5L).

### Methods

Pimavanserin 34 mg (provided as two 17 mg NUPLAZID® tablets) were administered orally as a single dose once daily.

Subjects were assessed for eligibility and prohibited medications were discontinued. After all screening assessments were completed, eligible subjects returned to the clinic for Baseline evaluation. After Baseline assessments were completed, subjects were enrolled and received the first dose of pimavanserin. Study medication were provided to the subjects to take home with instructions to take the medication approximately the same time each day, and return all used containers and unused study drug as scheduled.

At approximately 2, 4, and 6 weeks after start of dosing of study drug subjects returned to the clinic for assessments. Safety measures, HAMD-17, C-SSRS (Columbia Suicide Severity Rating Scale), CGI-S(Clinical Global Impression-Severity), and CGI-I (Clinical Global Impression-Improvement) were evaluated at all visits. Subjects completed the SCOPA, MMSE (Mini Mental State Examination), EQ-5D-5L, and UPDRS (Unified Parkinson's Disease Rating Scale) Part III at Baseline and Week 4.

At the end of the 8 weeks of study drug treatment, subjects returned to the clinic for their final clinical evaluation. All assessments were completed and unused study drug and containers were collected.

Approximately 2 weeks after the last dose of study drug, subjects had a safety follow-up telephone call.

### Subjects Selection

Subjects of age 50 years and older were selected for this study. Other eligibility criteria include the following:
The subject has a clinical diagnosis of idiopathic Parkinson's disease with a minimum duration of 1 year, defined as the presence of at least three of the following cardinal features, in the absence of alternative explanations or atypical features: 1) rest tremor 2) rigidity 3) bradykinesia and/or akinesia and 4) postural and gait abnormalities.
The subject meets clinical criteria for depression with Parkinson's disease as listed in the NINDS/NIHM Guidelines.
If currently taking an anti-depressant, the subject is being treated with only one SSRI or SNRI antidepressant as a dose within the US FDA-approved dose range. Patients who are currently taking a second antidepressant or antidepressant augmentation agent at a sub-therapeutic dose or for an inadequate duration at Screening, and can be discontinued from this agent before the Baseline visit (in the opinion of the Investigator) may be eligible for the study.
The subject is on a stable dose of anti-Parkinson's medication for 1 month prior to Screening.

Subjects who had a known personal or family history or symptoms of long QT syndrome were excluded from the study.

The HAMD-17 total score was analyzed using mixed model for repeated measures (MMRM). The model included effects for visit, Baseline HAMD-17 total score, and the Baseline HAMD-17 total score-by-visit interaction. An unstructured covariance matrix was used to model the within-subject errors and the Kenward-Roger approximation was used to adjust the denominator degrees of freedom. The treatment effect for the primary endpoint was estimated as the least-squares mean change from Baseline to Week 8, and was tested at a significance level of 0.05. In addition, the treatment effect was also estimated at each of the other time points (Weeks 2, 4, and 6) using the same MMRM model described above, and was considered secondary analyses.

Continuous secondary efficacy endpoints (CGI-I, CGI-S, SCOPA, and EQ-5D-5L) were analyzed using similar MMRM models as for the primary efficacy endpoint, except that the Baseline value of the endpoint being analyzed was included in the model as a covariate instead of the Baseline HAMD-17 total score. For CGI-I the response is the CGI-I score (as opposed to the change from Baseline), and the Baseline CGI-S score was used as the covariate.

For each of the continuous efficacy endpoints, in addition to MMRM, a paired t-test using a last observation carried forward imputation method was performed at each visit.

For the HAMD-17 responder endpoints (≥50% reduction from Baseline in HAMD-17 total score), the proportion of responders was summarized by visit, including 95% confidence intervals. Observed cases (subjects with missing values at a given visit are excluded) as well as missing values imputed as non-responders was presented.

### Safety Analyses

UPDRS Part III and MMSE was analyzed using similar MMRM models as for the primary efficacy endpoint, except that the Baseline value of the endpoint being analyzed (either UPDRS Part III or MMSE) was included as a covariate, and in the Baseline score-by-visit interaction term, instead of the HAMD-17 total score.

Adverse events were classified into standard terminology using the Medical Dictionary for Regulatory Activities (MedDRA). Treatment-emergent adverse events (TEAEs), TEAEs leading to discontinuation, TEAEs related to study drug, TEAEs by maximum severity, serious adverse events (SAEs), and SAEs related to study drug were summarized.

Descriptive statistics for ECG, vital signs and weight, C-SSRS, MMSE, UPDRS Part III, and clinical laboratory parameters, including changes from Baseline, were tabulated by time point. Additionally, categorical analyses were conducted on the incidence of subjects with prolonged QTc intervals and changes in QTc intervals in accordance with International Council for Harmonisation (ICH) guidelines.

### Results

Interim results based on the first 31 patients have been evaluated. 55.9% of patients are male and average age is 68.1 years, with 16 patients on adjunctive therapy and 15 on monotherapy. At baseline, patients had a mean (SE) HAMD-17 of 19.8 (0.6). Change from Baseline to Week 8 (least squares mean [LSM] [SE]) in the HAMD-17 was −10.7 (1.0) (95% CI; −12.7, −8.7; p<0.001), with significant improvement seen as early as Week 2 (−8.4 [1.0]; 95% CI; −10.5, −6.4; p<0.001). Significant improvement was seen in both treatment therapy categories. 45.2% of patients had ≥50% improvement in the HAMD-17 at Week 8, with 35.5% of patients reaching remission (HAMD-17≤7). 54.8% of patients had a score of 1 or 2 (very much improved or much improved) based on CGI-I. Significant improvement was seen in change from Baseline to Week 8 SCOPA-Global Sleep Quality, -Nighttime Sleep, and -Daytime Sleepiness: −1.0 (0.4) (95% CI; −1.7, −0.3; p=0.010), −2.1 (0.7) (95% CI; −3.6, −0.6; p=0.008), −2.1 (0.4) (95% CI; −3.0, −1.2; p<0.001) respectively. Safety results were consistent with prior studies, with 13 patients reporting adverse events the most common being falls, UTI, diarrhea, and nausea.

### Example 3: Receptor Profiles of Pimavanserin and Compounds with Antidepressant Activity

5-HT_{2A} receptors represent important targets for depression. A variety of studies have shown antidepressant activity from compounds with potent antagonist or inverse agonist activity at 5-HT_{2A} receptors, and to varying degrees 5-HT2c receptors, but low affinity to serotonin transporter, norepinephrine transporter, and dopamine transporter, either alone or when coadministered with SSRIs (Table 7). Pimavanserin, with its potent activity as a 5-HT_{2A} antagonist/inverse agonist and lesser activity as a 5-HT2c antagonist/inverse agonist, has a similar receptor profile to many compounds with antidepressant activity.

Current research continues to investigate novel molecular and cellular mechanisms of augmentation of antidepressant therapies. 5-HT_{2A} receptors represent important targets for depression. A variety of studies have shown antidepressant activity from compounds with potent antagonist or inverse agonist activity at 5-HT_{2A} receptors, and, to varying degrees, 5-HT2c receptors, but low affinity to serotonin transporter, norepinephrine transporter, and dopamine transporter. Antidepressant activity from these compounds has been found when taken alone or when coadministered with SSRIs, however, with some compounds (e.g., ritanserin, volinanserin, pruvanserin) their ability to alleviate SSRI antidepressant side effects such as sexual dysfunction is not known. Other compounds including mirtazeapine, mianserin, and trazodone have been used to treat SSRI induced sexual dysfunction. Mirtazeapine, mianserin, and trazodone, however, act on, e.g., 5-HT2C, 5-HT3, alpha2-adrenoreceptors and/or histamine H1 receptors. Hence, it not clear that their mechanism of action is by way of 5-HT_{2A} receptors. Moreover, such compounds can have their own undesired side effects (e.g., priapism).

TABLE 7
Receptor Profiles of Pimavanserin and Compounds with Antidepressant Activity

| Target | PIM | RIT | VOL | PRUV | PIP | KET | MIRT | MIAN | TRAZ |
|---|---|---|---|---|---|---|---|---|---|
| SERT | Low | Low | Low | Low | NA | Low | Low | Low | 350 |
| NET | Low | Low | Low | Low | NA | Low | Low | 70 | Low |
| DAT | Low | Low | Low | Low | NA | Low | Low | Low | Low |
| 5-HT_{2A} | 0.4 | 0.1 | 0.2 | 0.7 | 5 | 2 | 70 | 3 | 35 |
| 5-HT_{2C} | 16 | 3 | 125 | Low | 120 | 125 | 40 | 3 | 200 |
| Other | None | None | None | None | D4 (5) | α1 (15-20) | α2 (15-20) | α2 (4-20) | 5-HT_{1A} (40- |
| noteworthy | | | | | α2 (35) | | H1 (1) | H1 (1) | 100) |
| targets | | | | | | | 5-HT3 (10) | 5-HT3 (6) | α1 (10-40) |
| | | | | | | | | | α2 (100-500) |
| | | | | | | | | | H1 (30) |

Source: Data on file, except for pipamperone (Schotte et al., 1996, Psychopharmacology, 124(1-2): 57-73); trazodone and mianserin (PDSP Kᵢ database, see Roth et al., 2000, Neuroscientist., 6: 252-262; Nelson and Thomas, 1989, Biochem Pharmacol, 38(10): 1693-1695; Cusack et al., 1994. Psychopharmacology. 114 (4): 559-65; Owens et al., 1997. J. Pharmacol. Exp. Ther. 283 (3): 1305-22); and mirtazapine (Brayfield, in Martindale: The Complete Drug Reference. 38th ed. (Brayfiled, ed.) London, UK: Pharmaceutical Press. 2014; Wikström et al., 2002, J Med Chem., 45(15): 3280-3285; and Anttila and Leinonen, 2001, CNS Drug Reviews, 7(3): 249-264).
Abbreviations: α1 = alpha1 adrenergic receptor; α2 = alpha2 adrenergic receptor; DAT = dopamine transporter; H1 = histamine 1 receptor; KET = ketanserin; MIAN = mianserin; MIRT = mirtazapine; NA = not available; NET = norepinephrine transporter; PIM = pimavanserin; PIP = pipamperone; PRUV = pruvanserin; RIT = ritanserin; SERT = serotonin transporter; TRAZ = trazodone; VOL = volinanserin.
Note:
Values represent the affinity (Kᵢ) in nM of the indicated ligands and transporters/receptors. For “Other Noteworthy Targets,” Kᵢ values are provided in parentheses. “Low” denotes a Kᵢ > 1000 nM.

Because SSRIs and serotonin-norepinephrine reuptake inhibitors (SNRIs) elevate synaptic levels of 5-HT, they indirectly activate all 14 5-HT receptor subtypes including 5-HT_{1A}, 5-HT_{2A}, 5-HT_{2C} and 5-HT3 receptors. Efficacy of SSRIs is thought to be primarily mediated through activation of 5-HT_{1A} serotonin receptors (Samuels et al., 2015, Nat Neurosci., 18(11):1606-1616). Nonclinical studies have shown that activation of 5-HT_{2A} receptors has many depressogenic effects that are blocked by selective 5-HT_{2A} antagonists/inverse agonists (Rajkumar et al., 2009, Eur. J. Pharmacol., 608(1-3):32-41; Hemrick-Luecke and Evans, 2002, Neuropharmacology, 42(2):162-169; Vaidya et al., 1997, J. Neurosci. 17(8):2785-2795). In essence, 5-HT_{1A} and 5-HT_{2A} receptors appear to have functionally opposing roles with respect to resolution or exacerbation of depression.

### Example 4 Clinical Experience with Pimavanserin in MDD (Study ACP-103-042)

### Study Design

Study ACP-103-042 was a Phase 2, multicenter, randomized, double-blind, placebo-controlled, 2-stage SPCD study to evaluate the efficacy and safety of adjunctive pimavanserin in adult patients with inadequate treatment response to SSRI or SNRI antidepressant. Patients were required to be receiving a stable regimen of treatment with an SSRI/SNRI antidepressant as treatment for MDD to which they had exhibited an inadequate response. Each subject entering Stage 1 was randomly assigned (1:3) to receive either 34 mg pimavanserin (52 subjects) or placebo (155 subjects) once daily (QD). At the end of Stage 1 (Week 5), subjects initially randomized to placebo and who had met the criteria for a non-responder (i.e., HAMD-17 total score at Week 5>14 and a reduction from baseline in HAMD-17 total score of <50%) were randomly assigned (1:1) to pimavanserin 34 mg/day (29 subjects) or placebo (29 subjects). The determination of the subject's status and eligibility for randomization in Stage 2 was made in a double-blind manner. Subjects who did not meet criteria for randomization into Stage 2 continued with the assigned treatment from Stage 1 for an additional 5-week period (until the end of the study double-blind treatment).

Sexual functioning was assessed using the Massachusetts General Hospital Sexual Functioning Index or MGH-SFI (Labbate and Lare, 2001, Psychother Psychosom. 70(4):221-225). This is a patient facing questionnaire that quantifies sexual dysfunction into 5 functional domains (“interest in sex,” “sexual arousal,” “ability to achieve orgasm,” “ability to maintain erection” [malesonly], and “sexual satisfaction”). Patients rate each item using a 6-point scale ranging from 1 (good function) to 6 (poor function). The time frame for this scale is the past month. This scale was administered at baseline and Week 5 in both stages of the study. High scores indicate poor sexual functioning. In ACP-103-042 the mean scores for MGH-SFI at Baseline was calculated over all 5 domains for males and the 4 domains for females. For MGH-SFI (follow-up version) at Study Weeks 5 and 10, the mean was calculated in the same way as MGH-SFI at Baseline.

### Baseline Demographics

TABLE 8
Age and Sex Distribution in ACP-103-042

| | Stage 1 | | | | | | Stage 2 | | | | | |
|---|---|---|---|---|---|---|---|---|---|---|---|---|
| | Placebo | | PIM | | Total | | Placebo | | PIM | | Total | |
| | (n = 155) | | (n = 52) | | (n = 207) | | (n = 29) | | (n = 29) | | (n = 58) | |
| Sex, n(%) | 155 | | 52 | | 207 | | 29 | | 29 | | 58 | |
| Female | 108 | (69.7) | 43 | (82.7) | 151 | (72.9) | 22 | (75.9) | 20 | (69.0) | 42 | (72.4) |
| Male | 47 | (30.3) | 9 | (17.3) | 56 | (27.1) | 7 | (24.1) | 9 | (31.0) | 16 | (27.6) |
| Age (years) | | | | | | | | | | | | |
| n | 155 | | 52 | | 207 | | 29 | | 29 | | 58 | |
| Mean (SE) | 45.4 | (1.24) | 48.6 | (1.85) | 46.2 | (1.04) | 44.1 | (2.50) | 45.3 | (3.14) | 44.7 | (1.99) |
| Age Categories, | 155 | | 52 | | 207 | | 29 | | 29 | | 58 | |
| n(%) | | | | | | | | | | | | |
| 18 to 40 years | 55 | (35.5) | 14 | (26.9) | 69 | (33.3) | 9 | (31.0) | 11 | (37.9) | 20 | (34.5) |
| >40 years | 100 | (64.5) | 38 | (73.1) | 138 | (66.7) | 20 | (69.0) | 18 | (62.1) | 38 | (65.5) |

### Preliminary Efficacy Results

In the trial, pimavanserin met the primary endpoint of the weighted average results of Stage 1 and Stage 2, by overall significantly reducing Hamilton Rating Scale for Depression (17-Item) (HAMD-17) total score compared to placebo (p=0.0390). In Stage 1, positive improvement on HAMD-17 were achieved starting at week 1 (p=0.0365) and sustained through week 5 (p=0.0003) of the treatment period. Re-randomization of placebo non-responders from Stage 1 to Stage 2 did not yield further separation. On the key secondary endpoint, pimavanserin demonstrated overall statistically significant reductions compared to placebo in the Sheehan Disability Scale (SDS) score (p=0.0040). Overall positive results were also observed for other secondary endpoints including Clinical Global Impression-Improvement (CGI-I), Clinical Global Impression-Severity (CGI-S), Karolinska Sleepiness Scale (KSS) (p=0.02), Barratt Impulsiveness Scale (BIS-11), SF-12 Mental Component Summary (MCS), Massachusetts General Hospital Sexual Functioning Index MGH-SFI (p<0.001) as well as response rates, defined as a 50% or greater reduction on the HAMD-17 scale.

TABLE 9
Summary of Top-line Efficacy Results from ACP-103-042

| | Stage 1 | | | | Stage 2 | | | | |
|---|---|---|---|---|---|---|---|---|---|
| | Placebo | | Pimavanserin | | Placebo | | Pimavanserin | | |
| | (n = 152) | | (n = 51) | | (n = 29) | | (n = 29) | | Overall |
| Primary Endpoint | | | | | | | | | |
| HAMD-17 Item Score: LSM | −7.5 | (0.55) | −11.5 | (0.94) | −3.3 | (0.94) | −2.8 | (0.89) | |
| (SE) | | | | | | | | | |
| p-value | | | p = 0.0003 | | | | NS | | p = 0.0390 |
| Effect size (Cohen's d) | | | 0.626 | | | | | | |
| Secondary Endpoint | | | | | | | | | |
| SDS Score: LSM (SE) | −2.061 | (0.2040) | −3.254 | (0.3487) | −0.422 | (0.3025) | −0.901 | (0.2866) | |
| p-value | | | p = 0.0036 | | | | NS | | p = 0.004 |
| Effect size (Cohen's d) | | | 0.498 | | | | 0.311 | | |
| Other Secondary Endpoints¹ | | | | | | | | | |
| Response Rate, % (n) | 25.0% | (38) | 52.9% | (27) | 6.9% | (2) | 6.9% | (2) | |
| p-value | | | p = 0.0002 | | | | NS | | p = 0.0065 |
| Remission Rate, % (n) | 11.2% | (17) | 23.5% | (12) | 3.4% | (1) | 3.4% | (1) | |

| | | | |
|---|---|---|---|
| p-value | p = 0.0292 | NS | NS |
| CGI-I Score | p = 0.0010 | NS | p = 0.0289 |
| CGI-S Score | p = 0.0001 | NS | p = 0.0084 |
| KSS | p = 0.0003 | NS | p = 0.0205 |
| BIS-11 | NS | p = 0.0071 | p = 0.0075 |
| SF-12 MCS Score | p < 0.0001 | NS | p < 0.0001 |
| MGH-SFI | p = 0.0002 | NS | p = 0.0003 |

¹Additional secondary endpoints display nominal p-values

In the study Stage 1, the treatment effect size for pimavanserin compared to placebo on the Massachusetts General Hospital Sexual Functioning Index or MGH-SFI is 0.614 and this is considered to be large. In the study Stage 2, the treatment effect size for pimavanserin compared to placebo on the MGH-SFI is 0.412 and this is considered to be medium.

### MGH-SFI Scores: Overall and Subgroup by Age (<=50 and >50)

The treatment effect size for pimavanserin compared to placebo of the overall study group and subgroups by age (<=50 and >50) on the individual items of MGH-SFI were evaluated using stage-specific ANCOVA analysis. Tables 10-27 summarize the detailed data of MGH-SFI individual items of the overall study group (Tables 10-15), the subgroup of age of <=50 (Tables 16-21) and the subgroup of age of >50 (Table 21-26).

In Tables 9-26, ANCOVA=analysis of covariance; OC=observed cases; LSM=least squares mean; CI=confidence interval; SD=standard deviation; SE=standard error. [1] Stage 1: Baseline=Study Week 0, Week 5=Study Week 5; Stage 2: Baseline=Study Week 5, Week 5=Study Weeks 10. [2] LSM from the stage-specific ANCOVA analysis with the change from baseline as the outcome, treatment group as a factor, and the corresponding Baseline value as a covariate. [3] 2-sided p-value for treatment difference from the stage-specific ANCOVA analysis.

An treatment effect size (Cohen's d) is calculated using the formula: Effect Size=Least Square mean difference between PIM and PBO/SD. The corresponding 95% confidence interval for an effect size is calculated based on the theoretical probability distribution of the effect size. A positive treatment effect size (positive values on the x-axis) indicates that the pimavanserin arm performs better than the placebo arm in enhancing the sexual functioning measured by the individual items on the MGH-SFI. A negative treatment effect size (negative values on the x-axis) indicates that the placebo arm performs better than the pimavanserin arm. The effect size of value 0 represents that there is no difference between the pimavanserin arm and the placebo arm.

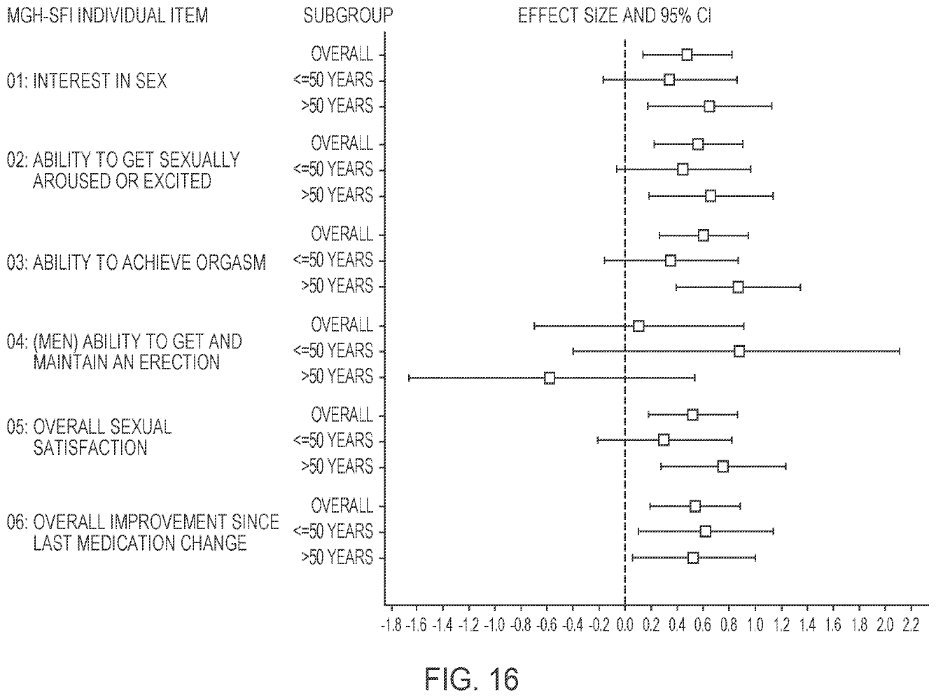

FIG. 16 summarizes the data in Tables 10-27. As shown in FIG. 16, in stage 1, pimavanserin significantly enhances the sexual functioning of the overall study group on the six individual items except for individual item 04 (Ability to Get and Maintain an Erection) by having a positive treatment effect size (positive values on the x-axis; on the right side of the value of 0 line (dotted line in the middle) as well as a positive 95% confidence interval (CI) of Effect Size (the lower limit of the interval is positive and on the right of the dotted line). For example, the overall study group has a positive treatment effect size in individual items Interest in Sex (0.483, p=0.0061), Ability to Get Sexually Aroused or Excited (0.560, p=0.0015), Ability to Achieve Orgasm (0.609, p=0.0006), Overall Sexual Satisfaction (0.524, p=0.0030) and Overall Improvement Since Last Medication Change (0.539, p=0.0023). As shown in FIG. 16, similar enhancing effects on these five individual items (Interest in Sex, Ability to Get Sexually Aroused or Excited, Ability to Achieve Orgasm, Overall Sexual Satisfaction and Overall Improvement Since Last Medication Change) are also observed in the subgroups of age of <=50 and age greater than 50.

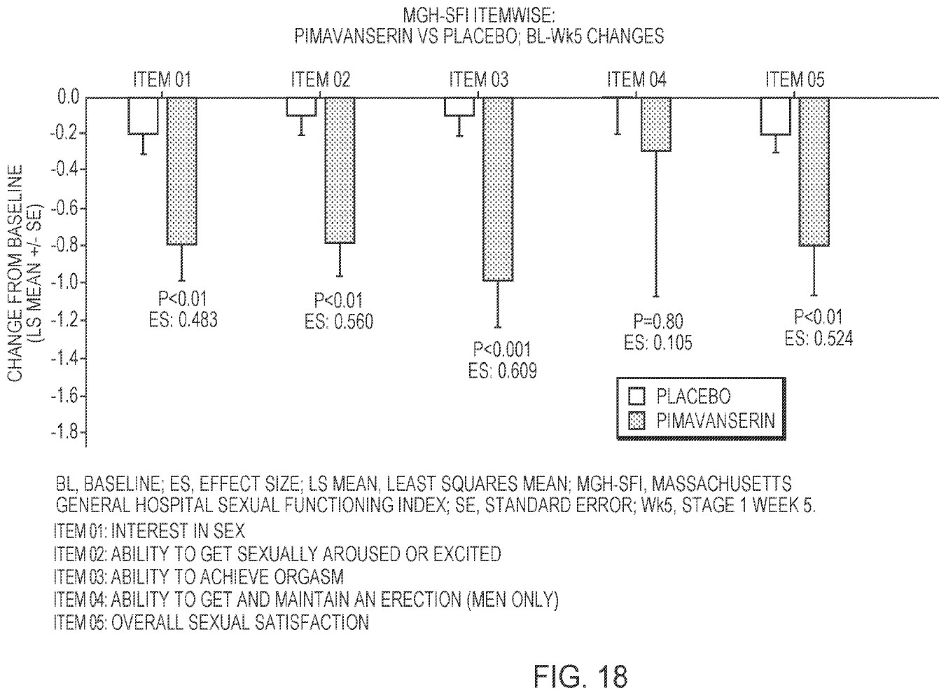

As shown in FIG. 18, the mean change from baseline to Week 5 for MGH-SFI Items 1, 2, 3, and 5 was significantly greater with pimavanserin vs. placebo.

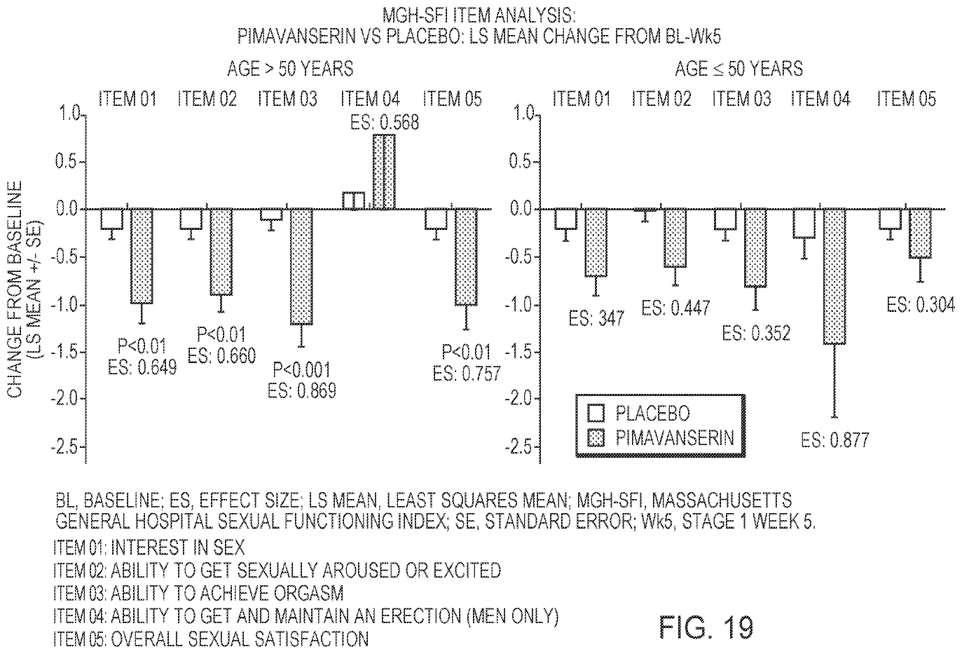

As shown in FIG. 19, the mean change for MGH-SFI Items stratified by age >50 or ≤50 years was greater with pimavanserin vs. placebo, but differences were only significant for the age >50 years subgroup.

TABLE 10
Analysis Data for Overall Study Group: Interest in Sex

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 152) | (N = 51) | (N = 29) | (N = 29) |
| 01: Interest in Sex | Week 5 | | | | |
| | n | 126 | 45 | 24 | 28 |
| | Mean (SE) | 4.3 (0.14) | 3.8 (0.27) | 4.1 (0.36) | 4.1 (0.28) |
| | SD | 1.52 | 1.79 | 1.75 | 1.50 |
| | Median | 5.0 | 4.0 | 4.5 | 4.0 |
| | Min, Max | 1, 6 | 1, 6 | 1, 6 | 1, 6 |
| | Change from Baseline | | | | |
| | to Week 5 | | | | |
| | n | 126 | 45 | 24 | 28 |
| | Mean (SE) | −0.2 (0.11) | −0.9 (0.23) | −0.3 (0.15) | −0.4 (0.16) |
| | SD | 1.29 | 1.56 | 0.74 | 0.84 |
| | Median | 0.0 | −1.0 | 0.0 | 0.0 |
| | Min, Max | −4, 3 | −5, 4 | −2, 1 | −3, 1 |
| | ANCOVA LSM (SE)[2] | −0.2 (0.11) | −0.8 (0.19) | −0.2 (0.16) | −0.4 (0.15) |
| | 95% CI of LSM | (−0.4, 0.0) | (−1.2, −0.5) | (−0.6, 0.1) | (−0.7, −0.1) |
| | Diff (PIM 34 mg-Placebo) in | | −0.6 (0.22) | | −0.2 (0.22) |
| | ANCOVA LSM (SE) | | | | |
| | 95% CI of Diff | | (−1.0, −0.2) | | (−0.6, 0.3) |
| | ANCOVA p-value[3] | | 0.0061 | | 0.4196 |
| | Effect Size (Cohen's d) | | 0.483 | | 0.227 |
| | 95% CI of Effect Size | | (0.137, 0.826) | | (−0.322, 0.772) |
| | Overall Treatment Comparison | | | | |
| | at Week 5 (Linear Combination | | | | |
| | Test) | | | | |
| | Weighted Diff in ANCOVA | | | | −0.4 (0.16) |
| | LSM (SE) | | | | |
| | 95% CI of Weighted Diff | | | | (−0.7, −0.1) |
| | p-value | | | | 0.0117 |

TABLE 11
Analysis Data for Overall Study Group: Ability to Get Sexually Aroused or Excited

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 152) | (N = 51) | (N = 29) | (N = 29) |
| 02: Ability to Get | Week 5 | | | | |
| Sexually Aroused | n | 126 | 45 | 24 | 28 |
| or Excited | Mean (SE) | 4.3 (0.13) | 3.7 (0.26) | 4.1 (0.35) | 4.2 (0.27) |
| | SD | 1.50 | 1.72 | 1.73 | 1.45 |
| | Median | 5.0 | 4.0 | 5.0 | 4.5 |
| | Min, Max | 1, 6 | 1, 6 | 1, 6 | 1, 6 |
| | Change from Baseline | | | | |
| | to Week 5 | | | | |
| | n | 126 | 45 | 24 | 28 |
| | Mean (SE) | −0.1 (0.11) | −0.8 (0.17) | −0.4 (0.16) | −0.4 (0.16) |
| | SD | 1.21 | 1.13 | 0.77 | 0.87 |
| | Median | 0.0 | −1.0 | 0.0 | 0.0 |
| | Min, Max | −4, 4 | −4, 1 | −3, 0 | −2, 1 |
| | ANCOVA LSM (SE)[2] | −0.1 (0.10) | −0.8 (0.17) | −0.4 (0.17) | −0.4 (0.16) |
| | 95% CI of LSM | (−0.3, 0.1) | (−1.1, −0.4) | (−0.7, 0.0) | (−0.7, 0.0) |
| | Diff (PIM 34 mg-Placebo) | | −0.6 (0.19) | | 0.0 (0.23) |
| | in ANCOVA LSM (SE) | | | | |
| | 95% CI of Diff | | (−1.0, −0.2) | | (−0.4, 0.5) |
| | ANCOVA p-value[3] | | 0.0015 | | 0.9295 |
| | Effect Size (Cohen's d) | | 0.560 | | −0.025 |
| | 95% CI of Effect Size | | (0.213, 0.904) | | (−0.570, 0.521) |
| | Overall Treatment Comparison | | | | |
| | at Week 5 (Linear Combination | | | | |
| | Test) | | | | |
| | Weighted Diff in | | | | −0.3 (0.15) |
| | ANCOVA LSM (SE) | | | | |
| | 95% CI of Weighted Diff | | | | (−0.6, 0.0) |
| | p-value | | | | 0.0458 |

TABLE 12
Analysis Data for Overall Study Group: Ability to Achieve Orgasm

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 152) | (N = 51) | (N = 29) | (N = 29) |
| 03: Ability to | Week 5 | | | | |
| Achieve Orgasm | n | 126 | 45 | 24 | 28 |
| | Mean (SE) | 4.3 (0.15) | 3.8 (0.26) | 4.4 (0.33) | 4.3 (0.32) |
| | SD | 1.66 | 1.72 | 1.64 | 1.71 |
| | Median | 5.0 | 4.0 | 5.0 | 5.0 |
| | Min, Max | 1, 6 | 1, 6 | 2, 6 | 1, 6 |
| | Change from Baseline | | | | |
| | to Week 5 | | | | |
| | n | 126 | 45 | 24 | 28 |
| | Mean (SE) | −0.1 (0.11) | −1.0 (0.24) | −0.2 (0.14) | −0.4 (0.18) |
| | SD | 1.28 | 1.64 | 0.70 | 0.96 |
| | Median | 0.0 | 0.0 | 0.0 | 0.0 |
| | Min, Max | −4, 4 | −4, 1 | −2, 1 | −3, 1 |
| | ANCOVA LSM (SE)[2] | −0.2 (0.11) | −0.9 (0.19) | −0.2 (0.17) | −0.4 (0.16) |
| | 95% CI of LSM | (−0.4, 0.1) | (−1.3, −0.6) | (−0.5, 0.2) | (−0.8, −0.1) |
| | Diff (PIM 34 mg-Placebo) | | −0.8 (0.22) | | −0.3 (0.24) |
| | in ANCOVA LSM (SE) | | | | |
| | 95% CI of Diff | | (−1.2, −0.3) | | (−0.7, 0.2) |
| | ANCOVA p-value[3] | | 0.0006 | | 0.2862 |
| | Effect Size (Cohen's d) | | 0.609 | | 0.300 |
| | 95% CI of Effect Size | | (0.258, 0.951) | | (−0.250, 0.847) |
| | Overall Treatment Comparison | | | | |
| | at Week 5 (Linear Combination | | | | |
| | Test) | | | | |
| | Weighted Diff in | | | | −0.5 (0.16) |
| | ANCOVA LSM (SE) | | | | |
| | 95% CI of Weighted Diff | | | | (−0.8, −0.2) |
| | p-value | | | | 0.0015 |

TABLE 13
Analysis Data for Overall Study Group: (Men) Ability to Get and Maintain an Erection

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 152) | (N = 51) | (N = 29) | (N = 29) |
| 04: (Men) Ability | Week 5 | | | | |
| to Get and | n | 36 | 7 | 5 | 9 |
| Maintain an | Mean (SE) | 3.7 (0.27) | 3.9 (0.70) | 3.0 (0.63) | 3.1 (0.54) |
| Erection | SD | 1.63 | 1.86 | 1.41 | 1.62 |
| | Median | 4.0 | 3.0 | 2.0 | 4.0 |
| | Min, Max | 1, 6 | 1, 6 | 2, 5 | 1, 5 |
| | Change from Baseline | | | | |
| | to Week 5 | | | | |
| | n | 36 | 7 | 5 | 9 |
| | Mean (SE) | 0.0 (0.20) | −0.3 (0.78) | 0.0 (0.32) | −1.0 (0.41) |
| | SD | 1.18 | 2.06 | 0.71 | 1.22 |
| | Median | 0.0 | 0.0 | 0.0 | −1.0 |
| | Min, Max | −3, 3 | −4, 2 | −1, 1 | −3, 1 |
| | ANCOVA LSM (SE)[2] | 0.0 (0.21) | −0.2 (0.48) | −0.2 (0.50) | −0.9 (0.37) |
| | 95% CI of LSM | (−0.5, 0.4) | (−1.1, 0.8) | (−1.3, 0.9) | (−1.7, −0.1) |
| | Diff (PIM 34 mg-Placebo) | | −0.1 (0.52) | | −0.8 (0.64) |
| | in ANCOVA LSM (SE) | | | | |
| | 95% CI of Diff | | (−1.2, 0.9) | | (−2.2, 0.7) |
| | ANCOVA p-value [3] | | 0.8018 | | 0.2655 |
| | Effect Size (Cohen's d) | | 0.105 | | 0.701 |
| | 95% CI of Effect Size | | (−0.706, 0.914) | | (−0.486, 1.767) |
| | Overall Treatment Comparison | | | | |
| | at Week 5 (Linear Combination | | | | |
| | Test) | | | | |
| | Weighted Diff in ANCOVA | | | | −0.4 (0.41) |
| | LSM (SE) | | | | |
| | 95% CI of Weighted Diff | | | | (−1.3, 0.4) |
| | p-value | | | | 0.2844 |

TABLE 14
Analysis Data for Overall Study Group: Overall Sexual Satisfaction

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 152) | (N = 51) | (N = 29) | (N = 29) |
| 05: Overall Sexual | Week 5 | | | | |
| Satisfaction | n | 126 | 45 | 24 | 28 |
| | Mean (SE) | 4.4 (0.14) | 3.9 (0.27) | 4.3 (0.38) | 4.1 (0.33) |
| | SD | 1.58 | 1.79 | 1.85 | 1.77 |
| | Median | 5.0 | 4.0 | 5.0 | 4.5 |
| | Min, Max | 1, 6 | 1, 6 | 1, 6 | 1, 6 |
| | Change from Baseline | | | | |
| | to Week 5 | | | | |
| | n | 126 | 45 | 24 | 28 |
| | Mean (SE) | −0.2 (0.10) | −0.8 (0.25) | −0.2 (0.25) | −0.7 (0.25) |
| | SD | 1.17 | 1.65 | 1.20 | 1.33 |
| | Median | 0.0 | 0.0 | 0.0 | −0.5 |
| | Min, Max | −4, 4 | −5, 3 | −3, 4 | −4, 1 |
| | ANCOVA LSM (SE)[2] | −0.2 (0.11) | −0.8 (0.18) | −0.2 (0.26) | −0.7 (0.24) |
| | 95% CI of LSM | (−0.4, 0.1) | (−1.2, −0.4) | (−0.7, 0.3) | (−1.2, −0.2) |
| | Diff (PIM 34 mg-Placebo) | | −0.6 (0.21) | | −0.5 (0.36) |
| | in ANCOVA LSM (SE) | | | | |
| | 95% CI of Diff | | (−1.1, −0.2) | | (−1.2, 0.2) |
| | ANCOVA p-value[3] | | 0.0030 | | 0.1568 |
| | Effect Size (Cohen's d) | | 0.524 | | 0.404 |
| | 95% CI of Effect Size | | (0.178, 0.868) | | (−0.153, 0.949) |
| | Overall Treatment Comparison | | | | |
| | at Week 5 (Linear Combination | | | | |
| | Test) | | | | |
| | Weighted Diff in | | | | −0.6 (0.21) |
| | ANCOVA LSM (SE) | | | | |
| | 95% CI of Weighted Diff | | | | (−1.0, −0.2) |
| | p-value | | | | 0.0055 |

TABLE 15
Analysis Data for Overall Study Group: Overall Improvement Since Last Medication Change

| | | Stage 1 | | Stage 2 | |
|---|---|---|---|---|---|
| MGH-SFI Individual | | Placebo | Pimavanserin | Placebo | Pimavanserin |
| Item | Visit[1] | (N = 152) | (N = 51) | (N = 29) | (N = 29) |
| 06: Overall Improvement | Week 5 | | | | |
| Since Last Medication | n | 124 | 45 | 22 | 28 |
| Change | Mean (SE) | 3.7 (0.08) | 3.2 (0.17) | 3.9 (0.20) | 3.6 (0.25) |
| | SD | 0.88 | 1.16 | 0.92 | 1.34 |
| | Median | 4.0 | 4.0 | 4.0 | 4.0 |
| | Min, Max | 1, 6 | 1, 5 | 2, 6 | 1, 6 |
| | ANCOVA LSM (SE)[2] | 3.7 (0.09) | 3.2 (0.14) | 3.9 (0.25) | 3.6 (0.22) |
| | 95% CI of LSM | (3.5, 3.9) | (2.9, 3.5) | (3.4, 4.4) | (3.2, 4.1) |
| | Diff (PIM 34 mg-Placebo) | | −0.5 (0.17) | | −0.3 (0.33) |
| | in ANCOVA LSM (SE) | | | | |
| | 95% CI of Diff | | (−0.8, −0.2) | | (−0.9, 0.4) |
| | ANCOVA p-value[3] | | 0.0023 | | 0.4303 |
| | Effect Size (Cohen's d) | | 0.539 | | 0.227 |
| | 95% CI of Effect Size | | (0.192, 0.884) | | (−0.335, 0.786) |
| | Overall Treatment Comparison | | | | |
| | at Week 5 (Linear Combination | | | | |
| | Test) | | | | |
| | Weighted Diff in | | | | −0.4 (0.19) |
| | ANCOVA LSM (SE) | | | | |
| | 95% CI of Weighted Diff | | | | (−0.8, 0.0) |
| | p-value | | | | 0.0362 |

TABLE 16
Analysis Data for Subgroup of <=50 Years: Interest in Sex

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 86) | (N = 20) | (N = 20) | (N = 15) |
| 01: Interest in Sex | Week 5 | | | | |
| | n | 69 | 19 | 15 | 15 |
| | Mean (SE) | 3.9 (0.19) | 3.5 (0.45) | 4.1 (0.47) | 3.9 (0.36) |
| | SD | 1.57 | 1.98 | 1.81 | 1.41 |
| | Median | 4.0 | 3.0 | 5.0 | 4.0 |
| | Min, Max | 1, 6 | 1, 6 | 1, 6 | 2, 6 |
| | Change from Baseline | | | | |
| | to Week 5 | | | | |
| | n | 69 | 19 | 15 | 15 |
| | Mean (SE) | −0.2 (0.16) | −0.7 (0.45) | −0.2 (0.22) | −0.3 (0.21) |
| | SD | 1.33 | 1.97 | 0.86 | 0.80 |
| | Median | 0.0 | 0.0 | 0.0 | 0.0 |
| | Min, Max | −4, 3 | −5, 4 | −2, 1 | −2, 1 |
| | ANCOVA LSM (SE)[2] | −0.2 (0.16) | −0.7 (0.31) | −0.2 (0.22) | −0.3 (0.22) |
| | 95% CI of LSM | (−0.5, 0.1) | (−1.3, 0.0) | (−0.6, 0.2) | (−0.7, 0.2) |
| | Diff (PIM 34 mg-Placebo) | | −0.5 (0.35) | | −0.1 (0.31) |
| | in ANCOVA LSM (SE) | | | | |
| | 95% CI of Diff | | (−1.2, 0.2) | | (−0.7, 0.6) |
| | ANCOVA p-value[3] | | 0.1848 | | 0.8270 |
| | Effect Size (Cohen's d) | | 0.347 | | 0.081 |
| | 95% CI of Effect Size | | (−0.165, 0.856) | | (−0.636, 0.796) |
| | Overall Treatment Comparison | | | | |
| | at Week 5 (Linear Combination | | | | |
| | Test) | | | | |
| | Weighted Diff in | | | | −0.3 (0.23) |
| | ANCOVA LSM (SE) | | | | |
| | 95% CI of Weighted Diff | | | | (−0.7, 0.2) |
| | p-value | | | | 0.2513 |

TABLE 17
Analysis Data for Subgroup of <=50 Years Old: Ability to Get Sexually Aroused or Excited

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 86) | (N = 20) | (N = 20) | (N = 15) |
| 02: Ability to Get | Week 5 | | | | |
| Sexually Aroused | n | 69 | 19 | 15 | 15 |
| or Excited | Mean (SE) | 4.1 (0.18) | 3.4 (0.42) | 4.3 (0.46) | 4.1 (0.33) |
| | SD | 1.53 | 1.84 | 1.79 | 1.28 |
| | Median | 4.0 | 3.0 | 5.0 | 4.0 |
| | Min, Max | 1, 6 | 1, 6 | 1, 6 | 2, 6 |
| | Change from Baseline | | | | |
| | to Week 5 | | | | |
| | n | 69 | 19 | 15 | 15 |
| | Mean (SE) | −0.1 (0.15) | −0.5 (0.27) | −0.3 (0.21) | −0.3 (0.25) |
| | SD | 1.28 | 1.17 | 0.82 | 0.98 |
| | Median | 0.0 | 0.0 | 0.0 | −1.0 |
| | Min, Max | −4, 4 | −4, 1 | −3, 0 | −2, 1 |
| | ANCOVA LSM (SE)[2] | 0.0 (0.14) | −0.6 (0.27) | −0.3 (0.24) | −0.3 (0.24) |
| | 95% CI of LSM | (−0.3, 0.2) | (−1.1, 0.0) | (−0.8, 0.2) | (−0.8, 0.1) |
| | Diff (PIM 34 mg-Placebo) | | −0.5 (0.30) | | 0.0 (0.33) |
| | in ANCOVA LSM (SE) | | | | |
| | 95% CI of Diff | | (−1.1, 0.1) | | (−0.7, 0.7) |
| | ANCOVA p-value[3] | | 0.0887 | | 0.9643 |
| | Effect Size (Cohen's d) | | 0.447 | | 0.017 |
| | 95% CI of Effect Size | | (−0.067, 0.957) | | (−0.699, 0.732) |
| | Overall Treatment Comparison | | | | |
| | at Week 5 (Linear Combination | | | | |
| | Test) | | | | |
| | Weighted Diff in ANCOVA | | | | −0.3 (0.22) |
| | LSM (SE) | | | | |
| | 95% CI of Weighted Diff | | | | (−0.7, 0.2) |
| | p-value | | | | 0.2339 |

TABLE 18
Analysis Data for Subgroup of <=50 Years Old: Ability to Achieve Orgasm

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 86) | (N = 20) | (N = 20) | (N = 15) |
| 03: Ability to | Week 5 | | | | |
| Achieve Orgasm | n | 69 | 19 | 15 | 15 |
| | Mean (SE) | 4.0 (0.20) | 3.8 (0.42) | 4.6 (0.42) | 4.1 (0.40) |
| | SD | 1.70 | 1.84 | 1.64 | 1.53 |
| | Median | 4.0 | 4.0 | 5.0 | 4.0 |
| | Min, Max | 1, 6 | 1, 6 | 2, 6 | 2, 6 |
| | Change from Baseline | | | | |
| | to Week 5 | | | | |
| | n | 69 | 19 | 15 | 15 |
| | Mean (SE) | −0.2 (0.17) | −0.8 (0.36) | 0.0 (0.17) | −0.3 (0.25) |
| | SD | 1.41 | 1.55 | 0.65 | 0.98 |
| | Median | 0.0 | 0.0 | 0.0 | 0.0 |
| | Min, Max | −4, 4 | −4, 1 | −2, 1 | −3, 1 |
| | ANCOVA LSM (SE)[2] | −0.2 (0.16) | −0.7 (0.31) | 0.0 (0.21) | −0.3 (0.21) |
| | 95% CI of LSM | (−0.5, 0.1) | (−1.3, −0.1) | (−0.4, 0.4) | (−0.8, 0.1) |
| | Diff (PIM 34 mg-Placebo) | | −0.5 (0.35) | | −0.4 (0.30) |
| | in ANCOVA LSM (SE) | | | | |
| | 95% CI of Diff | | (−1.2, 0.2) | | (−1.0, 0.2) |
| | ANCOVA p-value [3] | | 0.1808 | | 0.2303 |
| | Effect Size (Cohen's d) | | 0.352 | | 0.449 |
| | 95% CI of Effect Size | | (−0.162, 0.859) | | (−0.281, 1.170) |
| | Overall Treatment Comparison | | | | |
| | at Week 5 (Linear Combination | | | | |
| | Test) | | | | |
| | Weighted Diff in | | | | −0.4 (0.23) |
| | ANCOVA LSM (SE) | | | | |
| | 95% CI of Weighted Diff | | | | (−0.9, 0.0) |
| | p-value | | | | 0.0683 |

TABLE 19
Analysis Data for Subgroup of <=50 Years Old: (Men) Ability to Get and Maintain an Erection

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 86) | (N = 20) | (N = 20) | (N = 15) |
| 04: (Men) | Week 5 | | | | |
| Ability to Get | n | 18 | 3 | 2 | 4 |
| and Maintain | Mean (SE) | 3.1 (0.37) | 2.3 (0.67) | 2.0 (0.00) | 3.5 (0.50) |
| an Erection | SD | 1.55 | 1.15 | 0.00 | 1.00 |
| | Median | 3.0 | 3.0 | 2.0 | 4.0 |
| | Min, Max | 1, 6 | 1, 3 | 2, 2 | 2, 4 |
| | Change from Baseline | | | | |
| | to Week 5 | | | | |
| | n | 18 | 3 | 2 | 4 |
| | Mean (SE) | −0.3 (0.29) | −1.7 (1.45) | 0.5 (0.50) | −0.3 (0.48) |
| | SD | 1.23 | 2.52 | 0.71 | 0.96 |
| | Median | 0.0 | −2.0 | 0.5 | −0.5 |
| | Min, Max | −3, 2 | −4, 1 | 0, 1 | −1, 1 |
| | ANCOVA LSM (SE)[2] | −0.3 (0.29) | −1.4 (0.72) | −0.3 (0.96) | 0.2 (0.57) |
| | 95% CI of LSM | (−0.9, 0.3) | (−2.9, 0.1) | (−3.4, 2.7) | (−1.7, 2.0) |
| | Diff (PIM 34 mg-Placebo) | | −1.1 (0.78) | | 0.5 (1.33) |
| | in ANCOVA LSM (SE) | | | | |
| | 95% CI of Diff | | (−2.7, 0.6) | | (−3.8, 4.7) |
| | ANCOVA p-value[3] | | 0.1813 | | 0.7519 |
| | Effect Size (Cohen's d) | | 0.877 | | −0.526 |
| | 95% CI of Effect Size | | (−0.398, 2.110) | | (−1.990, 1.436) |
| | Overall Treatment Comparison | | | | |
| | at Week 5 (Linear Combination | | | | |
| | Test) | | | | |
| | Weighted Diff in ANCOVA | | | | −0.3 (0.77) |
| | LSM (SE) | | | | |
| | 95% CI of Weighted Diff | | | | (−1.8, 1.2) |
| | p-value | | | | 0.6844 |

TABLE 20
Analysis Data for Subgroup of <=50 Years Old: Overall Sexual Satisfaction

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 86) | (N = 20) | (N = 20) | (N = 15) |
| 05: Overall | Week 5 | | | | |
| Sexual | n | 69 | 19 | 15 | 15 |
| Satisfaction | Mean (SE) | 4.1 (0.20) | 3.6 (0.43) | 4.4 (0.49) | 4.0 (0.39) |
| | SD | 1.67 | 1.86 | 1.88 | 1.51 |
| | Median | 4.0 | 4.0 | 6.0 | 4.0 |
| | Min, Max | 1, 6 | 1, 6 | 1, 6 | 2, 6 |
| | Change from Baseline | | | | |
| | to Week 5 | | | | |
| | n | 69 | 19 | 15 | 15 |
| | Mean (SE) | −0.2 (0.16) | −0.5 (0.35) | 0.0 (0.38) | −0.6 (0.34) |
| | SD | 1.35 | 1.54 | 1.46 | 1.30 |
| | Median | 0.0 | 0.0 | 0.0 | −1.0 |
| | Min, Max | −4, 4 | −5, 2 | −3, 4 | −4, 1 |
| | ANCOVA LSM (SE)[2] | −0.2 (0.16) | −0.5 (0.30) | 0.0 (0.35) | −0.6 (0.35) |
| | 95% CI of LSM | (−0.5, 0.2) | (−1.1, 0.0) | (−0.7, 0.7) | (−1.3, 0.1) |
| | Diff (PIM 34 mg-Placebo) | | −0.4 (0.34) | | −0.5 (0.49) |
| | in ANCOVA LSM (SE) | | | | |
| | 95% CI of Diff | | (−1.1, 0.3) | | (−1.6, 0.5) |
| | ANCOVA p-value [3] | | 0.2438 | | 0.2786 |
| | Effect Size (Cohen's d) | | 0.304 | | 0.405 |
| | 95% CI of Effect Size | | (−0.207, 0.813) | | (−0.324, 1.124) |
| | Overall Treatment Comparison | | | | |
| | at Week 5 (Linear Combination | | | | |
| | Test) | | | | |
| | Weighted Diff in | | | | −0.5 (0.30) |
| | ANCOVA LSM (SE) | | | | |
| | 95% CI of Weighted Diff | | | | (−1.1, 0.1) |
| | p-value | | | | 0.1154 |

TABLE 21
Analysis Data for Subgroup of <=50 Years Old: Overall Improvement Since Last Medication Change

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 86) | (N = 20) | (N = 20) | (N = 15) |
| 06: Overall | Week 5 | | | | |
| Improvement | n | 68 | 19 | 13 | 15 |
| Since Last | Mean (SE) | 3.6 (0.12) | 3.0 (0.24) | 3.8 (0.22) | 3.8 (0.33) |
| Medication | SD | 1.01 | 1.05 | 0.80 | 1.26 |
| Change | Median | 4.0 | 3.0 | 4.0 | 4.0 |
| | Min, Max | 1, 6 | 1, 4 | 2, 5 | 2, 6 |
| | ANCOVA LSM (SE)[2] | 3.6 (0.12) | 3.0 (0.23) | 3.8 (0.30) | 3.8 (0.28) |
| | 95% CI of LSM | (3.4, 3.9) | (2.5, 3.5) | (3.2, 4.5) | (3.2, 4.4) |
| | Diff (PIM 34 mg-Placebo) | | −0.6 (0.26) | | 0.0 (0.41) |
| | in ANCOVA LSM (SE) | | | | |
| | 95% CI of Diff | | (−1.2, −0.1) | | (−0.9, 0.8) |
| | ANCOVA p-value[3] | | 0.0187 | | 0.9107 |
| | Effect Size (Cohen's d) | | 0.622 | | 0.043 |
| | 95% CI of Effect Size | | (0.103, 1.137) | | (−0.700, 0.785) |
| | Overall Treatment Comparison | | | | |
| | at Week 5 (Linear Combination | | | | |
| | Test) | | | | |
| | Weighted Diff in ANCOVA | | | | −0.3 (0.24) |
| | LSM (SE) | | | | |
| | 95% CI of Weighted Diff | | | | (−0.8, 0.1) |
| | p-value | | | | 0.1623 |

TABLE 22
Analysis Data for Subgroup of >50 Years: Interest in Sex

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 66) | (N = 31) | (N = 9) | (N = 14) |
| 01: Interest in Sex | Week 5 | | | | |
| | n | 57 | 26 | 9 | 13 |
| | Mean (SE) | 4.6 (0.18) | 3.9 (0.32) | 4.1 (0.59) | 4.4 (0.45) |
| | SD | 1.37 | 1.65 | 1.76 | 1.61 |
| | Median | 5.0 | 4.0 | 4.0 | 5.0 |
| | Min, Max | 2, 6 | 1, 6 | 2, 6 | 1, 6 |
| | Change from Baseline | | | | |
| | to Week 5 | | | | |
| | n | 57 | 26 | 9 | 13 |
| | Mean (SE) | −0.2 (0.16) | −1.0 (0.24) | −0.3 (0.17) | −0.6 (0.24) |
| | SD | 1.24 | 1.20 | 0.50 | 0.87 |
| | Median | 0.0 | −1.0 | 0.0 | 0.0 |
| | Min, Max | −4, 3 | −4, 1 | −1, 0 | −3, 0 |
| | ANCOVA LSM (SE)[2] | −0.2 (0.15) | −1.0 (0.22) | −0.3 (0.25) | −0.6 (0.21) |
| | 95% CI of LSM | (−0.6, 0.1) | (−1.4, −0.5) | (−0.8, 0.2) | (−1.1, −0.2) |
| | Diff (PIM 34 mg-Placebo) | | −0.7 (0.27) | | −0.3 (0.33) |
| | in ANCOVA LSM (SE) | | | | |
| | 95% CI of Diff | | (−1.3, −0.2) | | (−1.0, 0.3) |
| | ANCOVA p-value[3] | | 0.0075 | | 0.3049 |
| | Effect Size (Cohen's d) | | 0.649 | | 0.468 |
| | 95% CI of Effect Size | | (0.173, 1.122) | | (−0.411, 1.314) |
| | Overall Treatment Comparison | | | | |
| | at Week 5 (Linear Combination | | | | |
| | Test) | | | | |
| | Weighted Diff in ANCOVA | | | | −0.5 (0.21) |
| | LSM (SE) | | | | |
| | 95% CI of Weighted Diff | | | | (−1.0, −0.1) |
| | p-value | | | | 0.0106 |

TABLE 23
Analysis Data for Subgroup of >50 Years Old: Ability to Get Sexually Aroused or Excited

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 66) | (N = 31) | (N = 9) | (N = 14) |
| 02: Ability to Get | Week 5 | | | | |
| Sexually Aroused or | n | 57 | 26 | 9 | 13 |
| Excited | Mean (SE) | 4.6 (0.19) | 3.9 (0.32) | 3.9 (0.56) | 4.4 (0.46) |
| | SD | 1.41 | 1.63 | 1.69 | 1.66 |
| | Median | 5.0 | 4.0 | 4.0 | 5.0 |
| | Min, Max | 2, 6 | 2, 6 | 2, 6 | 1, 6 |
| | Change from Baseline | | | | |
| | to Week 5 | | | | |
| | n | 57 | 26 | 9 | 13 |
| | Mean (SE) | −0.2 (0.15) | −0.9 (0.21) | −0.4 (0.24) | −0.4 (0.21) |
| | SD | 1.12 | 1.09 | 0.73 | 0.77 |
| | Median | 0.0 | −1.0 | 0.0 | 0.0 |
| | Min, Max | −4, 3 | −4, 0 | −2, 0 | −2, 1 |
| | ANCOVA LSM (SE)[2] | −0.2 (0.14) | −0.9 (0.21) | −0.4 (0.26) | −0.4 (0.21) |
| | 95% CI of LSM | (−0.5, 0.1) | (−1.3, −0.5) | (−1.0, 0.1) | (−0.8, 0.1) |
| | Diff (PIM 34 mg-Placebo) | | −0.7 (0.25) | | 0.1 (0.34) |
| | in ANCOVA LSM (SE) | | | | |
| | 95% CI of Diff | | (−1.2, −0.2) | | (−0.6, 0.8) |
| | ANCOVA p-value [3] | | 0.0066 | | 0.8655 |
| | Effect Size (Cohen's d) | | 0.660 | | −0.075 |
| | 95% CI of Effect Size | | (0.183, 1.133) | | (−0.924, 0.777) |
| | Overall Treatment Comparison | | | | |
| | at Week 5 (Linear Combination | | | | |
| | Test) | | | | |
| | Weighted Diff in | | | | −0.3 (0.21) |
| | ANCOVA LSM (SE) | | | | |
| | 95% CI of Weighted Diff | | | | (−0.7, 0.1) |
| | p-value | | | | 0.1246 |

TABLE 24
Analysis Data for Subgroup of >50 Years Old: Ability to Achieve Orgasm

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 66) | (N = 31) | (N = 9) | (N = 14) |
| 03: Ability to | Week 5 | | | | |
| Achieve Orgasm | n | 57 | 26 | 9 | 13 |
| | Mean (SE) | 4.8 (0.20) | 3.8 (0.33) | 4.1 (0.56) | 4.5 (0.54) |
| | SD | 1.51 | 1.67 | 1.69 | 1.94 |
| | Median | 5.0 | 4.0 | 5.0 | 5.0 |
| | Min, Max | 2, 6 | 2, 6 | 2, 6 | 1, 6 |
| | Change from Baseline | | | | |
| | to Week 5 | | | | |
| | n | 57 | 26 | 9 | 13 |
| | Mean (SE) | −0.1 (0.15) | −1.2 (0.33) | −0.4 (0.24) | −0.5 (0.27) |
| | SD | 1.11 | 1.70 | 0.73 | 0.97 |
| | Median | 0.0 | 0.0 | 0.0 | 0.0 |
| | Min, Max | −4, 3 | −4, 1 | −2, 0 | −3, 0 |
| | ANCOVA LSM (SE)[2] | −0.1 (0.16) | −1.2 (0.24) | −0.4 (0.30) | −0.6 (0.25) |
| | 95% CI of LSM | (−0.4, 0.2) | (−1.7, −0.7) | (−1.0, 0.2) | (−1.1, 0.0) |
| | Diff (PIM 34 mg-Placebo) in | | −1.1 (0.29) | | −0.1 (0.39) |
| | ANCOVA LSM (SE) | | | | |
| | 95% CI of Diff | | (−1.7, −0.5) | | (−1.0, 0.7) |
| | ANCOVA p-value[3] | | 0.0004 | | 0.7049 |
| | Effect Size (Cohen's d) | | 0.869 | | 0.169 |
| | 95% CI of Effect Size | | (0.382, 1.347) | | (−0.687, 1.016) |
| | Overall Treatment Comparison | | | | |
| | at Week 5 (Linear Combination | | | | |
| | Test) | | | | |
| | Weighted Diff in ANCOVA | | | | −0.6 (0.24) |
| | LSM (SE) | | | | |
| | 95% CI of Weighted Diff | | | | (−1.1, −0.1) |
| | p-value | | | | 0.0120 |

TABLE 25
Analysis Data for Subgroup of >50 Years Old: (Men) Ability to Get and Maintain an Erection

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 66) | (N = 31) | (N = 9) | (N = 14) |
| 04: (Men) Ability to | Week 5 | | | | |
| Get and Maintain an | n | 18 | 4 | 3 | 5 |
| Election | Mean (SE) | 4.4 (0.34) | 5.0 (0.71) | 3.7 (0.88) | 2.8 (0.92) |
| | SD | 1.46 | 1.41 | 1.53 | 2.05 |
| | Median | 5.0 | 5.5 | 4.0 | 2.0 |
| | Min, Max | 2, 6 | 3, 6 | 2, 5 | 1, 5 |
| | Change from Baseline | | | | |
| | to Week 5 | | | | |
| | n | 18 | 4 | 3 | 5 |
| | Mean (SE) | 0.2 (0.26) | 0.8 (0.48) | −0.3 (0.33) | −1.6 (0.51) |
| | SD | 1.11 | 0.96 | 0.58 | 1.14 |
| | Median | 0.0 | 0.5 | 0.0 | −2.0 |
| | Min, Max | −1, 3 | 0, 2 | −1, 0 | −3, 0 |
| | ANCOVA LSM (SE)[2] | 0.2 (0.23) | 0.8 (0.49) | −0.3 (0.63) | −1.6 (0.49) |
| | 95% CI of LSM | (−0.3, 0.7) | (−0.3, 1.8) | (−1.9, 1.3) | (−2.9, −0.4) |
| | Diff (PIM 34 mg-Placebo) | | 0.6 (0.54) | | −1.3 (0.80) |
| | in ANCOVA LSM (SE) | | | | |
| | 95% CI of Diff | | (−0.6, 1.7) | | (−3.3, 0.8) |
| | ANCOVA p-value[3] | | 0.3175 | | 0.1721 |
| | Effect Size (Cohen's d) | | −0.568 | | 1.174 |
| | 95% CI of Effect Size | | (−1.658, 0.538) | | (−0.475, 2.712) |
| | Overall Treatment Comparison | | | | |
| | at Week 5 (Linear Combination | | | | |
| | Test) | | | | |
| | Weighted Diff in | | | | −0.4 (0.48) |
| | ANCOVA LSM (SE) | | | | |
| | 95% CI of Weighted Diff | | | | (−1.3, 0.6) |
| | p-value | | | | 0.4573 |

TABLE 26
Analysis Data for Subgroup of >50 Years Old: Overall Sexual Satisfaction

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 66) | (N = 31) | (N = 9) | (N = 14) |
| 05: Overall Sexual | Week 5 | | | | |
| Satisfaction | n | 57 | 26 | 9 | 13 |
| | Mean (SE) | 4.9 (0.18) | 4.1 (0.34) | 4.0 (0.62) | 4.2 (0.58) |
| | SD | 1.35 | 1.75 | 1.87 | 2.09 |
| | Median | 5.0 | 4.0 | 5.0 | 5.0 |
| | Min, Max | 2, 6 | 2, 6 | 1, 6 | 1, 6 |
| | Change from Baseline | | | | |
| | to Week 5 | | | | |
| | n | 57 | 26 | 9 | 13 |
| | Mean (SE) | −0.1 (0.12) | −1.1 (0.34) | −0.4 (0.18) | −0.8 (0.39) |
| | SD | 0.93 | 1.72 | 0.53 | 1.41 |
| | Median | 0.0 | 0.0 | 0.0 | 0.0 |
| | Min, Max | −4, 2 | −4, 3 | −1, 0 | −4, 1 |
| | ANCOVA LSM (SE)[2] | −0.2 (0.15) | −1.0 (0.23) | −0.3 (0.37) | −0.9 (0.31) |
| | 95% CI ofLSM | (−0.5, 0.2) | (−1.5, −0.6) | (−1.1, 0.4) | (−1.6, −0.3) |
| | Diff (PIM 34 mg-Placebo) | | −0.9 (0.28) | | −0.6 (0.49) |
| | in ANCOVA LSM (SE) | | | | |
| | 95% CI of Diff | | (−1.4, −0.3) | | (−1.6, 0.4) |
| | ANCOVA p-value[3] | | 0.0020 | | 0.2296 |
| | Effect Size (Cohen's d) | | 0.757 | | 0.555 |
| | 95% CI of Effect Size | | (0.275, 1.232) | | (−0.335, 1.398) |
| | Overall Treatment Comparison | | | | |
| | at Week 5 (Linear Combination | | | | |
| | Test) | | | | |
| | Weighted Diff in ANCOVA | | | | −0.7 (0.28) |
| | LSM (SE) | | | | |
| | 95% CI of Weighted Diff | | | | (−1.3, −0.2) |
| | p-value | | | | 0.0079 |

TABLE 27
Analysis Data for Subgroup of >50 Years Old: Overall Improvement Since Last Medication Change

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 66) | (N = 31) | (N = 9) | (N = 14) |
| 06: Overall | Week 5 | | | | |
| Improvement Since | n | 56 | 26 | 9 | 13 |
| Last Medication | Mean (SE) | 3.8 (0.09) | 3.3 (0.24) | 4.0 (0.37) | 3.5 (0.40) |
| Change | SD | 0.69 | 1.23 | 1.12 | 1.45 |
| | Median | 4.0 | 4.0 | 4.0 | 4.0 |
| | Min, Max | 2, 6 | 1, 5 | 2, 6 | 1, 6 |
| | ANCOVA LSM (SE)[2] | 3.8 (0.12) | 3.3 (0.18) | 4.0 (0.44) | 3.5 (0.37) |
| | 95% CI of LSM | (3.6, 4.1) | (3.0, 3.7) | (3.1, 4.9) | (2.7, 4.2) |
| | Diff (PIM 34 mg-Placebo) in | | −0.5 (0.21) | | −0.5 (0.58) |
| | ANCOVA LSM (SE) | | | | |
| | 95% CI of Diff | | (−0.9, −0.1) | | (−1.7, 0.7) |
| | ANCOVA p-value[3] | | 0.0280 | | 0.3606 |
| | Effect Size (Cohen's d) | | 0.531 | | 0.406 |
| | 95% CI of Effect Size | | (0.057, 1.002) | | (−0.458, 1.260) |
| | Overall Treatment Comparison | | | | |
| | at Week 5 (Linear Combination | | | | |
| | Test) | | | | |
| | Weighted Diff in ANCOVA | | | | −0.5 (0.31) |
| | LSM (SE) | | | | |
| | 95% CI of Weighted Diff | | | | (−1.1, 0.1) |
| | p-value | | | | 0.0984 |

### MGH-SFI Scores: Overall and Subgroup by Sex (Female and Male)

The treatment effect size for pimavanserin compared to placebo of the overall study group and subgroups by sex (female and male) on the individual items of MGH-SFI were evaluated using stage-specific ANCOVA analysis. Tables 10-15 and 28-38 summarize the detailed data of MGH-SFI individual items of the overall study group (Tables 10-15), the female subgroup (Tables 33-37) and the male subgroup (Table 38-43). An treatment effect size (Cohen's d) is calculated using the formula: Effect Size=Least Square mean difference between PIM and PBO/SD. The corresponding 95% confidence interval for an effect size is calculated based on the theoretical probability distribution of the effect size.

In Tables 10-15 and 28-38, ANCOVA=analysis of covariance; OC=observed cases; LSM=least squares mean; CI=confidence interval; SD=standard deviation; SE=standard error. [1] Stage 1: Baseline=Study Week 0, Week 5=Study Week 5; Stage 2: Baseline=Study Week 5, Week 5=Study Weeks 10. [2] LSM from the stage-specific ANCOVA analysis with the change from baseline as the outcome, treatment group as a factor, and the corresponding Baseline value as a covariate. [3] 2-sided p-value for treatment difference from the stage-specific ANCOVA analysis.

An treatment effect size (Cohen's d) is calculated using the formula: Effect Size=Least Square mean difference between PIM and PBO/SD. The corresponding 95% confidence interval for an effect size is calculated based on the theoretical probability distribution of the effect size. A positive treatment effect size (positive values on the x-axis) indicates that the pimavanserin arm performs better than the placebo arm in enhancing the sexual functioning measured by the individual items on the MGH-SFI. A negative treatment effect size (negative values on the x-axis) indicates that the placebo arm performs better than the pimavanserin arm. The effect size of value 0 represents that there is no difference between the pimavanserin arm and the placebo arm.

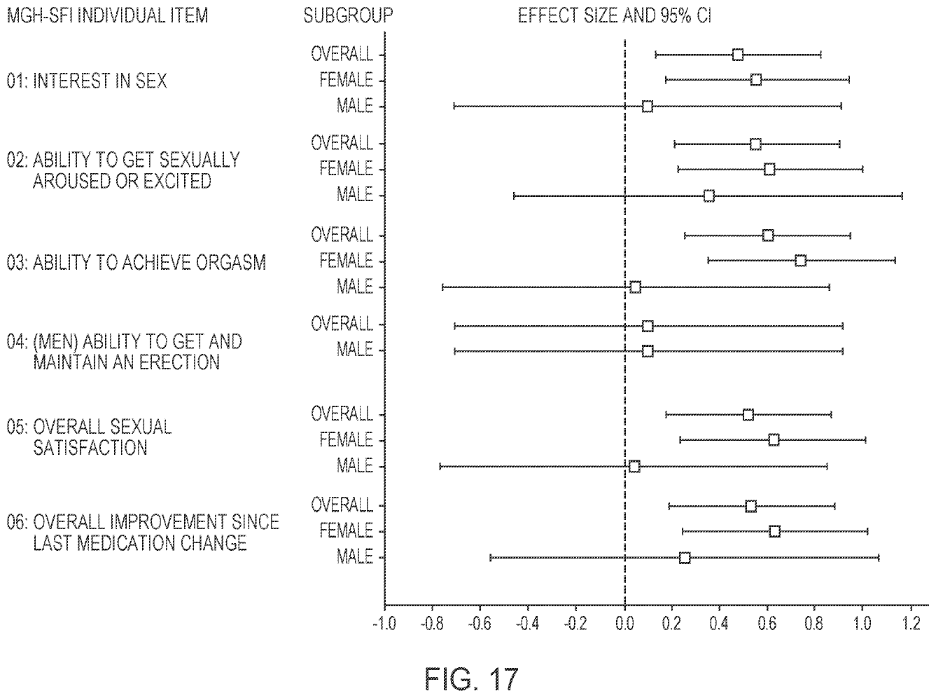

FIG. 17 summarizes the data in Tables 10-15 and 28-38. As shown in FIG. 17, in Stage 1, pimavanserin significantly enhances the sexual functioning on these five individual items (Interest in Sex, Ability to Get Sexually Aroused or Excited, Ability to Achieve Orgasm, Overall Sexual Satisfaction and Overall Improvement Since Last Medication Change) in the female subgroup. For example, the female group has a positive treatment effect size in individual items Interest in Sex (0.561, p=0.0044), Ability to Get Sexually Aroused or Excited (0.616, p=0.0019), Ability to Achieve Orgasm (0.749, p=0.0002), Overall Sexual Satisfaction (0.627, p=0.0015) and Overall Improvement Since Last Medication Change (0.634, p=0.0014).

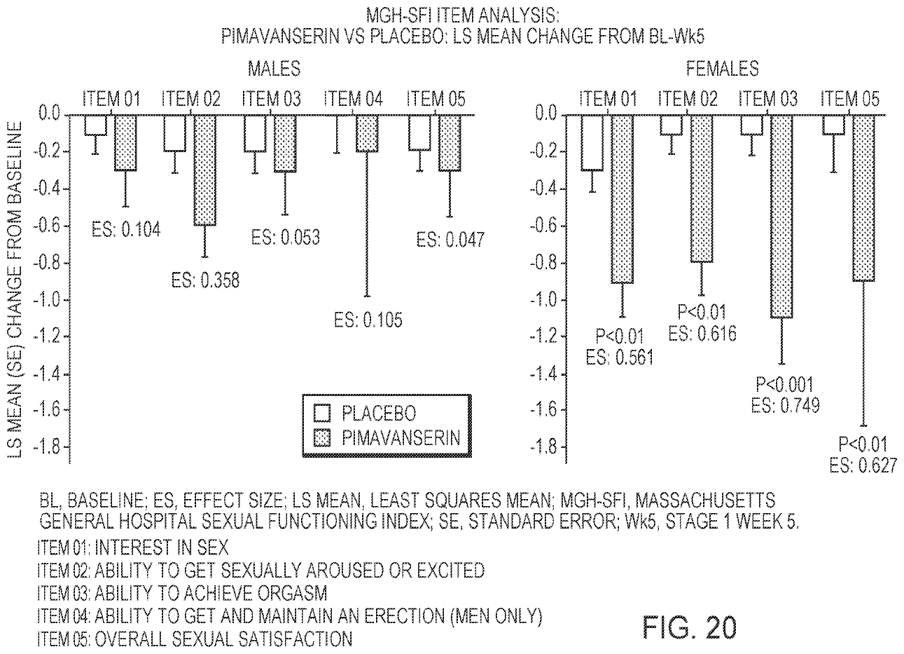

As shown in FIG. 20, the mean change from baseline to Week 5 for MGH-SFI Items were significantly greater with pimavanserin vs. placebo in female patients with effect sizes >0.5.

TABLE 28
Analysis Data for Female Subgroup: Interest in Sex

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 107) | (N = 43) | (N = 22) | (N = 20) |
| 01: Interest | Week 5 | | | | |
| in Sex | n | 90 | 38 | 19 | 19 |
| | Mean (SE) | 4.4 (0.16) | 3.7 (0.29) | 4.4 (0.40) | 4.4 (0.34) |
| | SD | 1.50 | 1.82 | 1.74 | 1.50 |
| | Median | 5.0 | 4.0 | 5.0 | 5.0 |
| | Min, Max | 2, 6 | 1, 6 | 1, 6 | 2, 6 |
| | Change from Baseline | | | | |
| | to Week 5 | | | | |
| | n | 90 | 38 | 19 | 19 |
| | Mean (SE) | −0.3 (0.13) | −0.9 (0.21) | −0.2 (0.16) | −0.3 (0.17) |
| | SD | 1.25 | 1.28 | 0.71 | 0.73 |
| | Median | 0.0 | −1.0 | 0.0 | 0.0 |
| | Min, Max | −4, 3 | −4, 2 | −2, 1 | −2, 1 |
| | ANCOVA LSM (SE)[2] | −0.3 (0.13) | −0.9 (0.19) | −0.2 (0.17) | −0.3 (0.17) |
| | 95% CI of LSM | (−0.5, 0.0) | (−1.3, −0.5) | (−0.5, 0.1) | (−0.6, 0.1) |
| | Diff (PIM 34 mg-Placebo) | | −0.7 (0.23) | | −0.1 (0.23) |
| | in ANCOVA LSM (SE) | | | | |
| | 95% CI of Diff | | (−1.1, −0.2) | | (−0.5, 0.4) |
| | ANCOVA p-value[3] | | 0.0044 | | 0.8240 |
| | Effect Size (Cohen's d) | | 0.561 | | 0.073 |
| | 95% CI of Effect Size | | (0.175, 0.946) | | (−0.564, 0.708) |
| | Overall Treatment Comparison | | | | |
| | at Week 5 (Linear Combination | | | | |
| | Test) | | | | |
| | Weighted Diff in | | | | −0.4 (0.16) |
| | ANCOVA LSM (SE) | | | | |
| | 95% CI of Weighted Diff | | | | (−0.7, 0.0) |
| | p-value | | | | 0.0285 |

TABLE 29
Analysis Data for Female Subgroup: Ability to Get Sexually Aroused or Excited

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 107) | (N = 43) | (N = 22) | (N = 20) |
| 02: Ability to Get | Week 5 | | | | |
| Sexually Aroused | n | 90 | 38 | 19 | 19 |
| or Excited | Mean (SE) | 4.5 (0.16) | 3.7 (0.28) | 4.4 (0.39) | 4.5 (0.32) |
| | SD | 1.47 | 1.73 | 1.71 | 1.39 |
| | Median | 5.0 | 3.5 | 5.0 | 5.0 |
| | Min, Max | 2, 6 | 1, 6 | 1, 6 | 2, 6 |
| | Change from Baseline | | | | |
| | to Week 5 | | | | |
| | n | 90 | 38 | 19 | 19 |
| | Mean (SE) | −0.1 (0.13) | −0.8 (0.17) | −0.3 (0.19) | −0.3 (0.20) |
| | SD | 1.19 | 1.05 | 0.82 | 0.87 |
| | Median | 0.0 | −1.0 | 0.0 | 0.0 |
| | Min, Max | −4, 4 | −4, 1 | −3, 0 | −2, 1 |
| | ANCOVA LSM (SE)[2] | −0.1 (0.12) | −0.8 (0.18) | −0.3 (0.20) | −0.3 (0.20) |
| | 95% CI of LSM | (−0.3, 0.1) | (−1.1, −0.4) | (−0.7, 0.1) | (−0.7, 0.1) |
| | Diff (PIM 34 mg-Placebo) in | | −0.7 (0.21) | | 0.1 (0.28) |
| | ANCOVA LSM (SE) | | | | |
| | 95% CI of Diff | | (−1.1, −0.3) | | (−0.5, 0.6) |
| | ANCOVA p-value [3] | | 0.0019 | | 0.8451 |
| | Effect Size (Cohen's d) | | 0.616 | | −0.064 |
| | 95% CI of Effect Size | | (0.227, 1.001) | | (−0.699, 0.573) |
| | Overall Treatment Comparison | | | | |
| | at Week 5 (Linear Combination | | | | |
| | Test) | | | | |
| | Weighted Diff in ANCOVA | | | | −0.3 (0.17) |
| | LSM (SE) | | | | |
| | 95% CI of Weighted Diff | | | | (−0.7, 0.0) |
| | p-value | | | | 0.0764 |

TABLE 30
Analysis Data for Female Subgroup: Ability to Achieve Orgasm

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 107) | (N = 43) | (N = 22) | (N = 20) |
| 03: Ability to | Week 5 | | | | |
| Achieve Orgasm | n | 90 | 38 | 19 | 19 |
| | Mean (SE) | 4.5 (0.17) | 3.8 (0.28) | 4.7 (0.35) | 4.6 (0.38) |
| | SD | 1.61 | 1.70 | 1.52 | 1.68 |
| | Median | 5.0 | 4.0 | 5.0 | 5.0 |
| | Min, Max | 2, 6 | 1, 6 | 2, 6 | 2, 6 |
| | Change from Baseline | | | | |
| | to Week 5 | | | | |
| | n | 90 | 38 | 19 | 19 |
| | Mean (SE) | −0.1 (0.13) | −1.2 (0.27) | −0.2 (0.18) | −0.3 (0.20) |
| | SD | 1.26 | 1.64 | 0.76 | 0.87 |
| | Median | 0.0 | 0.0 | 0.0 | 0.0 |
| | Min, Max | −4, 4 | −4, 1 | −2, 1 | −3, 1 |
| | ANCOVA LSM (SE)[2] | −0.1 (0.13) | −1.1 (0.21) | −0.2 (0.19) | −0.3 (0.19) |
| | 95% CI of LSM | (−0.4, 0.1) | (−1.5, −0.7) | (−0.5, 0.2) | (−0.6, 0.1) |
| | Diff (PIM 34 mg-Placebo) | | −1.0 (0.25) | | −0.1 (0.27) |
| | in ANCOVA LSM (SE) | | | | |
| | 95% CI of Diff | | (−1.4, −0.5) | | (−0.7, 0.4) |
| | ANCOVA p-value[3] | | 0.0002 | | 0.6875 |
| | Effect Size (Cohen's d) | | 0.749 | | 0.132 |
| | 95% CI of Effect Size | | (0.355, 1.136) | | (−0.506, 0.767) |
| | Overall Treatment Comparison | | | | |
| | at Week 5 (Linear Combination | | | | |
| | Test) | | | | |
| | Weighted Diff in | | | | −0.5 (0.18) |
| | ANCOVA LSM (SE) | | | | |
| | 95% CI of Weighted Diff | | | | (−0.9, −0.2) |
| | p-value | | | | 0.0036 |

TABLE 31
Analysis Data for Female Subgroup: Overall Sexual Satisfaction

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 107) | (N = 43) | (N = 22) | (N = 20) |
| 05: Overall Sexual | Week 5 | | | | |
| Satisfaction | n | 90 | 38 | 19 | 19 |
| | Mean (SE) | 4.6 (0.16) | 3.8 (0.29) | 4.5 (0.44) | 4.6 (0.38) |
| | SD | 1.56 | 1.79 | 1.90 | 1.64 |
| | Median | 5.0 | 3.5 | 6.0 | 5.0 |
| | Min, Max | 2, 6 | 1, 6 | 1, 6 | 2, 6 |
| | Change from Baseline | | | | |
| | to Week 5 | | | | |
| | n | 90 | 38 | 19 | 19 |
| | Mean (SE) | −0.1 (0.12) | −0.9 (0.26) | −0.1 (0.30) | −0.4 (0.26) |
| | SD | 1.14 | 1.57 | 1.31 | 1.12 |
| | Median | 0.0 | 0.0 | 0.0 | 0.0 |
| | Min, Max | −4, 4 | −4, 3 | −3, 4 | −4, 1 |
| | ANCOVA LSM (SE)[2] | −0.1 (0.13) | −0.9 (0.20) | −0.1 (0.28) | −0.3 (0.28) |
| | 95% CI of LSM | (−0.4, 0.1) | (−1.3, −0.5) | (−0.6, 0.5) | (−0.9, 0.2) |
| | Diff (PIM 34 mg-Placebo) in | | −0.8 (0.23) | | −0.3 (0.40) |
| | ANCOVA LSM (SE) | | | | |
| | 95% CI of Diff | | (−1.2, −0.3) | | (−1.1, 0.5) |
| | ANCOVA p-value[3] | | 0.0015 | | 0.4941 |
| | Effect Size (Cohen's d) | | 0.627 | | 0.227 |
| | 95% CI of Effect Size | | (0.239, 1.013) | | (−0.415, 0.861) |
| | Overall Treatment Comparison | | | | |
| | at Week 5 (Linear Combination | | | | |
| | Test) | | | | |
| | Weighted Diff in ANCOVA | | | | −0.5 (0.23) |
| | LSM (SE) | | | | |
| | 95% CI of Weighted Diff | | | | (−1.0, −0.1) |
| | p-value | | | | 0.0259 |

TABLE 32
Analysis Data for Female Subgroup: Overall Improvement Since Last Medication Change

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 107) | (N = 43) | (N = 22) | (N = 20) |
| 06: Overall | Week 5 | | | | |
| Improvement | n | 89 | 38 | 17 | 19 |
| Since Last | Mean (SE) | 3.8 (0.10) | 3.2 (0.17) | 3.8 (0.25) | 3.6 (0.29) |
| Medication | SD | 0.90 | 1.06 | 1.01 | 1.26 |
| Change | Median | 4.0 | 4.0 | 4.0 | 4.0 |
| | Min, Max | 1, 6 | 1, 4 | 2, 6 | 2, 6 |
| | ANCOVA LSM (SE)[2] | 3.8 (0.10) | 3.2 (0.15) | 3.8 (0.28) | 3.6 (0.26) |
| | 95% CI of LSM | (3.6, 4.0) | (2.9, 3.5) | (3.3, 4.4) | (3.1, 4.2) |
| | Diff (PIM 34 mg-Placebo) in | | −0.6 (0.18) | | −0.2 (0.38) |
| | ANCOVA LSM (SE) | | | | |
| | 95% CI of Diff | | (−1.0, −0.2) | | (−1.0, 0.6) |
| | ANCOVA p-value[3] | | 0.0014 | | 0.6200 |
| | Effect Size (Cohen's d) | | 0.634 | | 0.167 |
| | 95% CI of Effect Size | | (0.245, 1.021) | | (−0.490, 0.821) |
| | Overall Treatment Comparison | | | | |
| | at Week 5 (Linear Combination | | | | |
| | Test) | | | | |
| | Weighted Diff in ANCOVA | | | | −0.4 (0.21) |
| | LSM (SE) | | | | |
| | 95% CI of Weighted Diff | | | | (−0.8, 0.0) |
| | p-value | | | | 0.0619 |

TABLE 33
Analysis Data for Male Subgroup: Interest in Sex

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 45) | (N = 8) | (N = 7) | (N = 9) |
| 01: Interest in Sex | Week 5 | | | | |
| | n | 36 | 7 | 5 | 9 |
| | Mean (SE) | 4.0 (0.26) | 4.1 (0.63) | 3.0 (0.63) | 3.6 (0.47) |
| | SD | 1.57 | 1.68 | 1.41 | 1.42 |
| | Median | 4.0 | 5.0 | 2.0 | 4.0 |
| | Min, Max | 1, 6 | 1, 6 | 2, 5 | 1, 5 |
| | Change from Baseline | | | | |
| | to Week 5 | | | | |
| | n | 36 | 7 | 5 | 9 |
| | Mean (SE) | −0.1 (0.23) | −0.6 (1.04) | −0.4 (0.40) | −0.8 (0.32) |
| | SD | 1.38 | 2.76 | 0.89 | 0.97 |
| | Median | 0.0 | −1.0 | −1.0 | −1.0 |
| | Min, Max | −4, 3 | −5, 4 | −1, 1 | −3, 0 |
| | ANCOVA LSM (SE)[2] | −0.1 (0.23) | −0.3 (0.54) | −0.5 (0.43) | −0.7 (0.31) |
| | 95% CI of LSM | (−0.6, 0.3) | (−1.4, 0.8) | (−1.5, 0.4) | (−1.4, 0.0) |
| | Diff (PIM 34 mg-Placebo) | | −0.1 (0.59) | | −0.2 (0.54) |
| | in ANCOVA LSM (SE) | | | | |
| | 95% CI of Diff | | (−1.3, 1.0) | | (−1.4, 1.0) |
| | ANCOVA p-value[3] | | 0.8045 | | 0.7731 |
| | Effect Size (Cohen's d) | | 0.104 | | 0.174 |
| | 95% CI of Effect Size | | (−0.708, 0.912) | | (−0.934, 1.256) |
| | Overall Treatment Comparison | | | | |
| | at Week 5 (Linear Combination | | | | |
| | Test) | | | | |
| | Weighted Diff in | | | | −0.2 (0.40) |
| | ANCOVA LSM (SE) | | | | |
| | 95% CI of Weighted Diff | | | | (−0.9, 0.6) |
| | p-value | | | | 0.7013 |

TABLE 34
Analysis Data for Male Subgroup: Ability to Get Sexually Aroused or Excited

| | | Stage 1 | | Stage 2 | |
|---|---|---|---|---|---|
| MGH-SFI | | Placebo | Pimavanserin | Placebo | Pimavanserin |
| Individual Item | Visit[l] | (N = 45) | (N = 8) | (N = 7) | (N = 9) |
| 02: Ability to Get | Week 5 | | | | |
| Sexually Aroused | n | 36 | 7 | 5 | 9 |
| or Excited | Mean (SE) | 3.9 (0.25) | 3.7 (0.68) | 3.0 (0.63) | 3.6 (0.47) |
| | SD | 1.51 | 1.80 | 1.41 | 1.42 |
| | Median | 4.0 | 4.0 | 2.0 | 4.0 |
| | Min, Max | 1, 6 | 1, 6 | 2, 5 | 1, 5 |
| | Change from Baseline | | | | |
| | to Week 5 | | | | |
| | n | 36 | 7 | 5 | 9 |
| | Mean (SE) | −0.1 (0.21) | −0.7 (0.61) | −0.6 (0.24) | −0.6 (0.29) |
| | SD | 1.27 | 1.60 | 0.55 | 0.88 |
| | Median | 0.0 | 0.0 | −1.0 | −1.0 |
| | Min, Max | −4, 3 | −4, 1 | −1, 0 | −2, 1 |
| | ANCOVA LSM (SE)[2] | −0.2 (0.20) | −0.6 (0.45) | −0.6 (0.36) | −0.5 (0.27) |
| | 95% CI of LSM | (−0.6, 0.2) | (−1.5, 0.3) | (−1.4, 0.1) | (−1.1, 0.1) |
| | Diff (PIM 34 mg-Placebo) in | | −0.4 (0.49) | | 0.1 (0.45) |
| | ANCOVA LSM (SE) | | | | |
| | 95% CI of Diff | | (−1.4, 0.6) | | (−0.9, 1.1) |
| | ANCOVA p-value[3] | | 0.3935 | | 0.7910 |
| | Effect Size (Cohen's d) | | 0.358 | | −0.154 |
| | 95% CI of Effect Size | | (−0.459, 1.167) | | (−1.243, 0.947) |
| | Overall Treatment Comparison | | | | |
| | at Week 5 (Linear Combination | | | | |
| | Test) | | | | |
| | Weighted Diff in ANCOVA | | | | −0.2 (0.33) |
| | LSM (SE) | | | | |
| | 95% CI of Weighted Diff | | | | (−0.8, 0.5) |
| | p-value | | | | 0.6488 |

TABLE 35
Analysis Data for Male Subgroup: Ability to Achieve Orgasm

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 45) | (N = 8) | (N = 7) | (N = 9) |
| 03: Ability to | Week 5 | | | | |
| Achieve Orgasm | n | 36 | 7 | 5 | 9 |
| | Mean (SE) | 3.8 (0.28) | 4.1 (0.74) | 3.2 (0.73) | 3.6 (0.56) |
| | SD | 1.70 | 1.95 | 1.64 | 1.67 |
| | Median | 4.0 | 5.0 | 2.0 | 4.0 |
| | Min, Max | 1, 6 | 1, 6 | 2, 5 | 1, 6 |
| | Change from Baseline | | | | |
| | to Week 5 | | | | |
| | n | 36 | 7 | 5 | 9 |
| | Mean (SE) | −0.2 (0.22) | −0.4 (0.61) | −0.2 (0.20) | −0.8 (0.36) |
| | SD | 1.35 | 1.62 | 0.45 | 1.09 |
| | Median | 0.0 | 0.0 | 0.0 | 0.0 |
| | Min, Max | −4, 3 | −4, 1 | −1, 0 | −3, 0 |
| | ANCOVA LSM (SE)[2] | −0.2 (0.22) | −0.3 (0.50) | −0.3 (0.44) | −0.7 (0.32) |
| | 95% CI of LSM | (−0.7, 0.2) | (−1.3, 0.7) | (−1.2, 0.7) | (−1.5, 0.0) |
| | Diff (PIM 34 mg-Placebo) in | | −0.1 (0.55) | | −0.5 (0.56) |
| | ANCOVA LSM (SE) | | | | |
| | 95% CI of Diff | | (−1.2, 1.0) | | (−1.7, 0.8) |
| | ANCOVA p-value[3] | | 0.8993 | | 0.4131 |
| | Effect Size (Cohen's d) | | 0.053 | | 0.497 |
| | 95% CI of Effect Size | | (−0.757, 0.862) | | (−0.646, 1.575) |
| | Overall Treatment Comparison | | | | |
| | at Week 5 (Linear Combination | | | | |
| | Test) | | | | |
| | Weighted Diff in ANCOVA | | | | −0.3 (0.39) |
| | LSM (SE) | | | | |
| | 95% CI of Weighted Diff | | | | (−1.0, 0.5) |
| | p-value | | | | 0.4864 |

TABLE 36
Analysis Data for Male Subgroup: (Men) Ability to Get and Maintain an Erection

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 45) | (N = 8) | (N = 7) | (N = 9) |
| 04: (Men) Ability to | Week 5 | | | | |
| Get and Maintain an | n | 36 | 7 | 5 | 9 |
| Erection | Mean(SE) | 3.7 (0.27) | 3.9 (0.70) | 3.0 (0.63) | 3.1 (0.54) |
| | SD | 1.63 | 1.86 | 1.41 | 1.62 |
| | Median | 4.0 | 3.0 | 2.0 | 4.0 |
| | Min, Max | 1, 6 | 1, 6 | 2, 5 | 1, 5 |
| | Change from Baseline | | | | |
| | to Week 5 | | | | |
| | n | 36 | 7 | 5 | 9 |
| | Mean (SE) | 0.0 (0.20) | −0.3 (0.78) | 0.0 (0.32) | −1.0 (0.41) |
| | SD | 1.18 | 2.06 | 0.71 | 1.22 |
| | Median | 0.0 | 0.0 | 0.0 | −1.0 |
| | Min, Max | −3, 3 | −4, 2 | −1, 1 | −3, 1 |
| | ANCOVA LSM (SE)[2] | 0.0 (0.21) | −0.2 (0.48) | −0.2 (0.50) | −0.9 (0.37) |
| | 95% CI of LSM | (−0.5, 0.4) | (−1.1, 0.8) | (−1.3, 0.9) | (−1.7, −0.1) |
| | Diff (PIM 34 mg-Placebo) | | −0.1 (0.52) | | −0.8 (0.64) |
| | in ANCOVA LSM (SE) | | | | |
| | 95% CI of Diff | | (−1.2, 0.9) | | (−2.2, 0.7) |
| | ANCOVA p-value[3] | | 0.8018 | | 0.2655 |
| | Effect Size (Cohen's d) | | 0.105 | | 0.701 |
| | 95% CI of Effect Size | | (−0.706, 0.914) | | (−0.486, 1.767) |
| | Overall Treatment Comparison | | | | |
| | at Week 5 (Linear Combination | | | | |
| | Test) | | | | |
| | Weighted Diff in ANCOVA | | | | −0.4 (0.41) |
| | LSM (SE) | | | | |
| | 95% CI of Weighted Diff | | | | (−1.3, 0.4) |
| | p-value | | | | 0.2844 |

TABLE 37
Analysis Data for Male Subgroup: Overall Sexual Satisfaction

| | | Stage 1 | | Stage 2 | |
|---|---|---|---|---|---|
| MGH-SFI | | Placebo | Pimavanserin | Placebo | Pimavanserin |
| Individual Item | Visit[1] | (N = 45) | (N = 8) | (N = 7) | (N = 9) |
| 05: Overall Sexual | Week 5 | | | | |
| Satisfaction | n | 36 | 7 | 5 | 9 |
| | Mean (SE) | 4.1 (0.26) | 4.6 (0.69) | 3.2 (0.58) | 3.0 (0.53) |
| | SD | 1.58 | 1.81 | 1.30 | 1.58 |
| | Median | 4.0 | 5.0 | 3.0 | 3.0 |
| | Min, Max | 1, 6 | 1, 6 | 2, 5 | 1, 5 |
| | Change from Baseline | | | | |
| | to Week 5 | | | | |
| | n | 36 | 7 | 5 | 9 |
| | Mean (SE) | −0.2 (0.21) | −0.6 (0.81) | −0.6 (0.24) | −1.4 (0.50) |
| | SD | 1.28 | 2.15 | 0.55 | 1.51 |
| | Median | 0.0 | 0.0 | −1.0 | −1.0 |
| | Min, Max | −4, 2 | −5, 2 | −1, 0 | −4, 1 |
| | ANCOVA LSM (SE)[2] | −0.2 (0.22) | −0.3 (0.50) | −0.7 (0.58) | −1.4 (0.43) |
| | 95% CI of LSM | (−0.7, 0.2) | (−1.3, 0.7) | (−2.0, 0.5) | (−2.3, −0.4) |
| | Diff (PIM 34 mg-Placebo) | | −0.1 (0.55) | | −0.6 (0.73) |
| | in ANCOVA LSM (SE) | | | | |
| | 95% CI of Diff | | (−1.2, 1.0) | | (−2.3, 1.0) |
| | ANCOVA p-value[3] | | 0.9119 | | 0.3947 |
| | Effect Size (Cohen's d) | | 0.047 | | 0.511 |
| | 95% CI of Effect Size | | (−0.764, 0.855) | | (−0.629, 1.595) |
| | Overall Treatment Comparison | | | | |
| | at Week 5 (Linear Combination | | | | |
| | Test) | | | | |
| | Weighted Diff in ANCOVA | | | | −0.4 (0.46) |
| | LSM (SE) | | | | |
| | 95% CI of Weighted Diff | | | | (−1.2, 0.5) |
| | p-value | | | | 0.4377 |

TABLE 38
Analysis Data for Male Subgroup: Overall Improvement Since Last Medication Change

| | | Stage 1 | | Stage 2 | |
|---|---|---|---|---|---|
| MGH-SFI | | Placebo | Pimavanserin | Placebo | Pimavanserin |
| Individual Item | Visit[l] | (N = 45) | (N = 8) | (N = 7) | (N = 9) |
| 06: Overall | Week 5 | | | | |
| Improvement | n | 35 | 7 | 5 | 9 |
| Since Last | Mean (SE) | 3.5 (0.14) | 3.3 (0.64) | 4.2 (0.20) | 3.7 (0.53) |
| Medication | SD | 0.82 | 1.70 | 0.45 | 1.58 |
| Change | Median | 4.0 | 3.0 | 4.0 | 4.0 |
| | Min, Max | 1, 5 | 1, 5 | 4, 5 | 1, 5 |
| | ANCOVA LSM (SE)[2] | 3.5 (0.17) | 3.3 (0.38) | 4.2 (0.59) | 3.7 (0.44) |
| | 95% CI of LSM | (3.2, 3.9) | (2.5, 4.1) | (2.9, 5.5) | (2.7, 4.6) |
| | Diff (PIM 34 mg-Placebo) in | | −0.3 (0.41) | | −0.5 (0.73) |
| | ANCOVA LSM (SE) | | | | |
| | 95% CI of Diff | | (−1.1, 0.6) | | (−2.1, 1.1) |
| | ANCOVA p-value [3] | | 0.5387 | | 0.4816 |
| | Effect Size (Cohen's d) | | 0.257 | | 0.405 |
| | 95% CI of Effect Size | | (−0.558, 1.069) | | (−0.708, 1.502) |
| | Overall Treatment Comparison | | | | |
| | at Week 5 (Linear Combination | | | | |
| | Test) | | | | |
| | Weighted Diff in ANCOVA | | | | −0.4 (0.42) |
| | LSM (SE) | | | | |
| | 95% CI of Weighted Diff | | | | (−1.2, 0.4) |
| | p-value | | | | 0.3486 |

### MDD Remission and Response Status

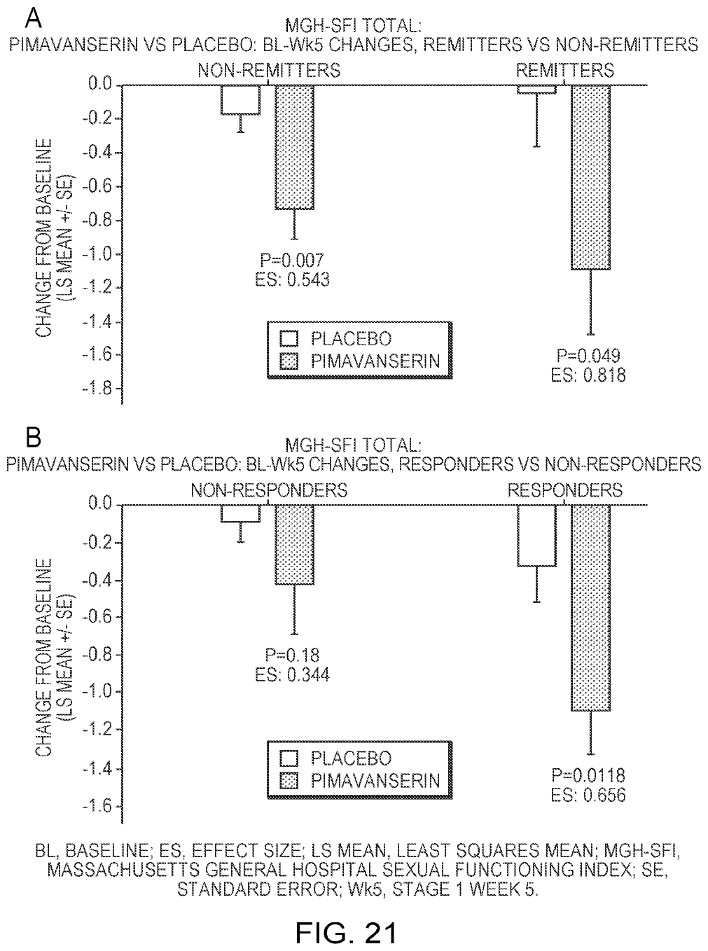

As shown in FIG. 21, pimavanserin demonstrated robust and significant effects vs. placebo on the MGH-SFI when assessed by MDD remission status. Similar findings were observed when assessed by MDD response status. FIG. 21 shows LS Mean (SE) change from baseline to week 5 for MGH-SFI Mean Score A) among patients with or without remission during Stage 1 only; and B) among patients with or without response during Stage 1 only.

### HAMD Item 14 (Sexual Interest)

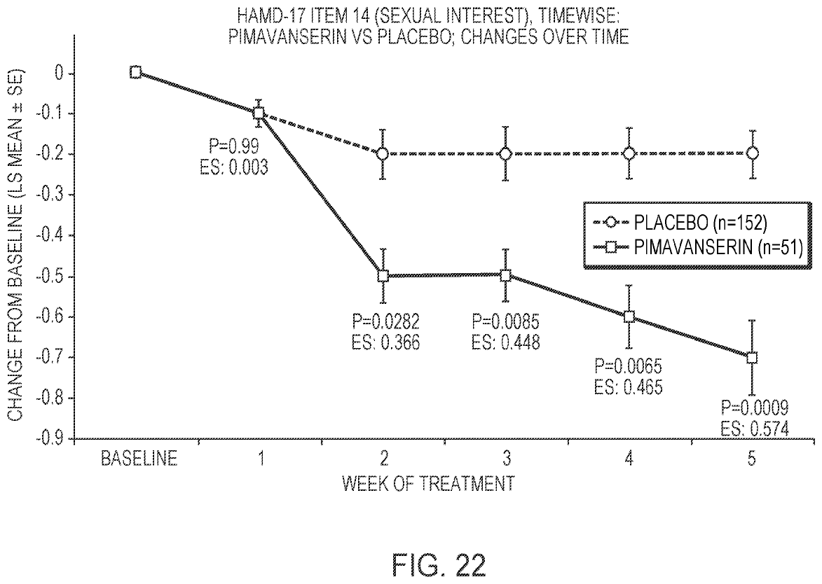

At Week 5, the LS Mean (SE) change from baseline for the HAMD-17 Item 14 (sexual interest) was −0.7 (0.12) for pimavanserin and −0.2 (0.07) for placebo (LS Mean difference: −0.5 [0.13]; 95% CI: −0.7, −0.2; P=0.0009; Cohen's d effect size: 0.574). The LS Mean difference was significant (P<0.05) from Weeks 2 through 5 As shown in FIG. 22.

### Preliminary Safety Results

In the study, pimavanserin was generally well-tolerated. One subject in each of pimavanserin and placebo groups reported serious adverse events, deemed not related by investigator to study drug. These resolved and both these subjects completed the study. Discontinuations due to adverse events were similar between pimavanserin (1.9 or 1.2%) and placebo (1.9 or 3.2%). There were no deaths reported in the study or other significant or unexpected safety findings.

Conclusions from Study ACP-103-042

In this study Pimavanserin was shown to be significantly more effective when compared to placebo on the primary endpoint HAMD-17 which measures depressive symptoms, the SDS which measures functioning and quality of life and on other secondary measures. Among these there was a significant improvement with medium to large and highly significant treatment effects on sexual functioning among those subjects treated with adjunctive pimavanserin compared to placebo as measured by the MGH-SFI. This finding supports that pimavanserin improves, e.g., (1) SSRI or SNRI-induced sexual dysfunction and (2) sexual dysfunction associated with major depressive disorder.

Although the invention has been described with reference to embodiments and examples, it should be understood that numerous and various modifications can be made without departing from the spirit of the invention. Accordingly, the invention is limited only by the following claims. All publications, patents, and patent applications cited in this specification are incorporated herein by reference as if each such publication, patent or patent application were specifically and individually indicated to be incorporated herein by reference.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A method of treating anxious distress with major depressive disorder in a patient in need thereof, comprising
orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

2. A method of treating major depressive disorder in a patient in need thereof, comprising
orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

3. The method of claim 2, wherein the patient also suffers from anxious distress.

4. The method of claim 2 or 3, wherein, after 5 weeks of daily administration, the patient has an improved score relative to the baseline in one or more of the Hamilton Depression Scale individual items selected from the group consisting of psychic anxiety, somatic anxiety, gastrointestinal symptoms, general somatic symptoms, work and interest, and hypochondriasis.

5. A method of treating treatment-resistant major depressive disorder in a patient in need thereof, comprising
orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

6. The method of any one of claims 1-5, wherein the patient has a DSM-5 defined diagnosis of major depressive disorder.

7. The method of any one of claims 1-6, wherein the patient has anxious distress during a majority of days of a major depressive disorder episode.

8. The method of any one of claims 1-7, wherein the patient is being treated with an SSRI or SNRI.

9. The method of claim 8, wherein the patient has an inadequate response to the SSRI or SNRI alone.

10. The method of claim 9, wherein the inadequate response is determined by MGH ATRQ (Antidepressant Treatment Response Questionnaire).

11. The method of any one of claims 8-10, wherein the SSRI or SNRI is selected from the group consisting of: citalopram, escitalopram, paroxetine, fluoxetine, sertraline, duloxetine, venlafaxine, desvenlafaxine, and fluvoxamine.

12. The method of claim 11, wherein the SSRI is paroxetine.

13. The method of any one of claims 1-12, wherein the anxious distress is selected from any one of the group consisting of separation anxiety disorder, selective mutism, specific phobia, social anxiety disorder, panic disorder, panic attack specifier, agoraphobia, generalized anxiety disorder, substance/medication-induced anxiety disorder, anxiety disorder due to another medical condition, post-traumatic stress disorder, and obsessive compulsive disorder.

14. The method of claim 13, wherein the anxious distress is generalized anxiety disorder and the patient is being treated with paroxetine, escitalopram, duloxetine, or venlafaxine.

15. The method of claim 13, wherein the anxious distress is post-traumatic stress disorder and the patient is being treated with sertraline or paroxetine.

16. The method of claim 13, wherein the anxious distress is obsessive compulsive disorder and the patient is being treated with fluoxetine, fluvoxamine, paroxetine, or sertraline.

17. The method of claim 13, wherein the anxious distress is panic disorder, and the patient is being treated with fluoxetine, paroxetine, sertraline, or venlafaxine.

18. The method of any one of claims 1-17, wherein the patient has a MADRS (Montgomery-Åsberg Depression Rating Scale) score of at least 20, at least 22, or at least 24 before the administration.

19. The method of claim 18, wherein the patient has a MADRS score of at least 20 before the administration.

20. The method of any one of claims 1-19, wherein the patient has a CGI-S score of at least 4 before the administration.

21. The method of any one of claims 1-20, wherein after 5 weeks of daily administration, the patient has a 50% or greater improvement in a HAMD-17 score relative to the baseline.

22. The method of any one of claims 1-21, wherein after 5 weeks of daily administration, the patient has a HAMD-17 score of less than 14.

23. The method of any one of claims 1-22, wherein after 5 weeks of daily administration, the patient has an improved Sheehan Disability Score (SDS) relative to the baseline.

24. The method of any one of claims 1-23, wherein the treating results in increased functional remission compared to a patient treated with a placebo.

25. The method of claim 24, wherein the functional remission is a total SDS score of 6 or less.

26. The method of any one of claims 1-25, wherein after 5 weeks of daily administration, the patient has an improved score in the work/school item of the SDS.

27. The method of any one of claims 1-26, wherein after 5 weeks of daily administration, the patient has an improved score in the social life item of the SDS.

28. The method of any one of claims 1-27, wherein after 5 weeks of daily administration, the patient has an improved score in the family life/home responsibilities item of the SDS.

29. The method of any one of claims 1-28, wherein after 5 weeks of daily administration, the patient has a reduced number of the days unproductive item of the SDS.

30. The method of any one of claims 1-29, wherein after 5 weeks of daily administration, the patient has a higher improvement in a HAMD-17 score than a patient treated with an SSRI or SNRI alone.

31. The method of any one of claims 1-30, wherein the patient has an early improvement as measured by Hamilton Depression Scale within 7 days of daily administration compared to a patient treated with a placebo.

32. The method of any one of claims 1-31, wherein after 5 weeks of daily administration, the patient has an improved score in the psychic anxiety item of the Hamilton Depression Scale.

33. The method of any one of claims 1-32, wherein after 5 weeks of daily administration, the patient has an improved score in the somatic anxiety item of the Hamilton Depression Scale.

34. The method of any one of claims 1-33, wherein after 5 weeks of daily administration, the patient has an improved score in the work and interest item of the Hamilton Depression Scale.

35. The method of any one of claims 1-34, wherein after 5 weeks of daily administration, the patient has an improved score in the gastrointestinal symptoms item of the Hamilton Depression Scale.

36. The method of any one of claims 1-35, wherein after 5 weeks of daily administration, the patient has an improved score in the general somatic symptoms item of the Hamilton Depression Scale.

37. The method of any one of claims 1-36, wherein the patient has an improved score in the hypochondria item of the Hamilton Depression Scale.

38. A method of treating melancholic features with major depressive disorder in a patient in need thereof, comprising
orally administering daily to the patient a compound selected from the group consisting of N-(4-fluorophenylmethyl)-N-(1-methylpiperidin-4-yl)-N′-(4-(2-methylpropyloxy)phenylmethyl)carbamide and pharmaceutically acceptable salts thereof.

39. The method of claim 38, wherein before the administration, the patient wakes up 2 hours earlier than usual.

40. The method of claim 38 or 39, wherein before the administration, the patient has observable psychomotor retardation or agitation.

41. The method of any one of claims 38-40, wherein before the administration, the patient has significant weight loss or anorexia.

42. The method of any one of claims 38-41, wherein the patient is also being treated with an SSRI or SNRI.

43. The method of claim 42, wherein the SSRI or SNRI is selected from the group consisting of: citalopram, escitalopram, paroxetine, fluoxetine, sertraline, duloxetine, venlafaxine, desvenlafaxine, and fluvoxamine.

44. The method of claim 43, wherein the SSRI is paroxetine.

45. The method of any one of claims 38-44, wherein the patient has had an inadequate response to SSRI or SNRI treatment alone.

46. The method of any one of claims 38-45, wherein after 5 weeks of daily administration, the patient has a 50% or greater improvement in a HAMD-6 score relative to the baseline, wherein the HAMD-6 consists of the following individual items: depressed mood, guilt feelings, work and interests, psychomotor retardation, psychic anxiety, and general somatic symptoms.

47. The method of any one of claims 38-46, wherein after 5 weeks of daily administration, the patient has a higher improvement in a HAMD-6 score than a patient treated with an SSRI or SNRI alone, wherein the HAMD-6 consists of the following individual items: depressed mood, guilt feelings, work and interests, psychomotor retardation, psychic anxiety, and general somatic symptoms.

48. The method of any one of claims 38-47, wherein after 5 weeks of daily administration, the patient has an improved score in the depressed mood item of the Hamilton Depression Scale.

49. The method of any one of claims 38-48, wherein after 5 weeks of daily administration, the patient has an improved score in the guilt item of the Hamilton Depression Scale.

50. The method of any one of claims 38-49, wherein after 5 weeks of daily administration, the patient has an improved score in the work and interest item of the Hamilton Depression Scale.

51. The method of any one of claims 38-50, wherein after 5 weeks of daily administration, the patient has an improved score in the psychomotor retardation item of the Hamilton Depression Scale.

52. The method of any one of claims 38-51, wherein after 5 weeks of daily administration, the patient has an improved score in the gastrointestinal symptoms item of the Hamilton Depression Scale.

53. The method of any one of claims 38-52, wherein after 5 weeks of daily administration, the patient has improved daytime sleepiness and/or improved nighttime sleep disturbance.

54. The method of claim 53, wherein the improved sleep disturbance is measured by at least a −1.5 change from baseline of the Karolinska Sleepiness Scale.

55. A method of treating generalized anxiety disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

56. A method of treating a somatic symptom disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

57. A method of treating an illness anxiety disorder in a patient in need thereof, comprising orally administering once daily to the patient an effective amount of a compound selected from N-(4-fluorophenylmethyl)-N-(1-methylpiperidin-4-yl)-N′-(4-(2-methylpropyloxy)phenylmethyl)carbamide or a pharmaceutically acceptable salt thereof.

58. The method of any one of claims 55-57, wherein the patient is also being treated with an SSRI or SNRI.

59. The method of claim 58, wherein the SSRI or SNRI is selected from the group consisting of: citalopram, escitalopram, paroxetine, fluoxetine, sertraline, duloxetine, venlafaxine, desvenlafaxine, and fluvoxamine.

60. The method of claim 58 or 59, wherein the patient has had an inadequate response to SSRI or SNRI treatment alone.

61. The method of any one of claims 55-60, wherein after 5 weeks of daily administration, the patient has an improved score in the psychic anxiety item of the Hamilton Depression Scale.

62. The method of any one of claims 55-61, wherein after 5 weeks of daily administration, the patient has an improved score in the somatic anxiety item of the Hamilton Depression Scale.

63. The method of any one of claims 55-62, wherein after 5 weeks of daily administration, the patient has an improved score in the work and interest item of the Hamilton Depression Scale.

64. The method of any one of claims 1-63, wherein N-(4-fluorophenylmethyl)-N-(1-methylpiperidin-4-yl)-N′-(4-(2-methylpropyloxy)phenylmethyl)carbamide is administered in the form of a tartrate salt.

65. The method of any one of claims 1-63, wherein orally administering comprises administering about 5 mg to about 40 mg, based on the free base form, of N-(4-fluorophenylmethyl)-N-(1-methylpiperidin-4-yl)-N′-(4-(2-methylpropyloxy)phenylmethyl)carbamide daily.

66. The method of any one of claims 1-65, wherein orally administering comprises administering 34 mg based on the free base form of N-(4-fluorophenylmethyl)-N-(1-methylpiperidin-4-yl)-N′-(4-(2-methylpropyloxy)phenylmethyl)carbamide daily.

67. The method of claim 66, wherein the 34 mg is administered in one capsule or two tablets (2×17 mg tablets).

68. The method of any one of claims 1-67, wherein the patient is concurrently being administered a CYP3A4 inhibitor, wherein orally administering comprises administering 10 mg of N-(4-fluorophenylmethyl)-N-(1-methylpiperidin-4-yl)-N′-(4-(2-methylpropyloxy)phenylmethyl)carbamide daily.

69. A method of treating anxious distress in a patient diagnosed with depression, comprising orally administering about 34 mg, once daily to the patient an effective amount of N-(4-fluorophenylmethyl)-N-(1-methylpiperidin-4-yl)-N′-(4-(2-methylpropyloxy)phenylmethyl)carbamide.

70. The method of claim 69, wherein the depression is major depressive disorder.

71. The method of claim 69 or 70, wherein the depression is treatment resistant depression.

72. A method of treating depression in a patient in need thereof, wherein the patient is also suffering from Parkinson's disease, comprising
orally administering to the patient 34 mg of N-(4-fluorophenylmethyl)-N-(1-methylpiperidin-4-yl)-N′-(4-(2-methylpropyloxy)phenylmethyl)carbamide once daily.

73. The method of claim 72, wherein after 8 weeks of administration, the patient has an improvement in depression symptoms as measured by the Hamilton Depression Scale.

74. The method of claim 73, wherein the patient has suffered from Parkinson's disease for one year or more.

75. The method of any one of claims 72-74, wherein the patient is 50 years or older.

76. The method of any one of claims 72-75, wherein treating results in the patient having a Hamilton Depression Scale-17 items (HAMD-17) score of less than or equal to 7.

77. The method of any one of claims 72-76, wherein treating results in the patient having an improved Scale of Outcomes in PD-Sleep (SCOPA) daytime score.

78. The method of any one of claims 72-77, wherein N-(4-fluorophenylmethyl)-N-(1-methylpiperidin-4-yl)-N′-(4-(2-methylpropyloxy)phenylmethyl)carbamide is administered in the form of a tartrate salt.

79. A method of treating depression associated with Parkinson's disease in an adult patient in need thereof comprising administering 34 mg of N-(4-fluorophenylmethyl)-N-(1-methylpiperidin-4-yl)-N′-(4-(2-methylpropyloxy)phenylmethyl)carbamide.

80. The method of any one of claims 72-79, wherein the patient is concurrently being administering an SSRI or SNRI.

81. A method for the treatment of major depressive disorder comprising the administration of a therapeutically effective amount of pimavanserin or a pharmaceutically acceptable salt thereof to a patient in need thereof, wherein said patient has previously received a SSRI or a SRNI or is concurrently also being administered a SSRI or a SRNI for the treatment of the major depressive disorder and the SSRI or the SRNI has caused sexual dysfunction.

82. The method of claim 81, wherein the patient is concurrently being administered the SSRI or the SRNI for the treatment of the major depressive disorder.

83. The method of claim 81 or 82, wherein the patient is a male patient.

84. The method of claim 81 or 82, wherein the patient is a female patient.

85. The method of any one of claims 81-84, wherein the sexual dysfunction is loss of interest in sex, loss of sexual arousal, diminished ability or inability to achieve orgasm, loss of ability to maintain an erection in male subjects, loss of sexual satisfaction, reduced frequency of sexual activity, or reduced satisfaction from sexual activity.

86. A method for the treatment of major depressive disorder comprising the administration of a therapeutically effective amount of pimavanserin or a pharmaceutically acceptable salt thereof to a patient in need thereof, wherein said patient has previously received a SSRI or a SRNI or is concurrently also being administered a SSRI or a SRNI for the treatment of the major depressive disorder and the SSRI or the SRNI has caused gastrointestinal or sleep related adverse events.

87. The method of any one of claims 81-86, wherein the patient has had an inadequate response to the SSRI or the SNRI alone.

88. The method of any one of claims 81-87, wherein the patient has a DSM-5 defined diagnosis of major depressive disorder.

89. The method of any one of claims 81-88, wherein the SSRI or SNRI is selected from the group consisting of: citalopram, escitalopram, paroxetine, fluoxetine, sertraline, duloxetine, venlafaxine, desvenlafaxine, and fluvoxamine.

90. The method of any one of claims 81-89, wherein N-(4-fluorophenylmethyl)-N-(1-methylpiperidin-4-yl)-N′-(4-(2-methylpropyloxy)phenylmethyl)carbamide is administered in the form of a tartrate salt.

91. The method of any one of claims 81-90, wherein orally administering comprises administering about 5 mg to about 40 mg, based on the free base form, of N-(4-fluorophenylmethyl)-N-(1-methylpiperidin-4-yl)-N′-(4-(2-methylpropyloxy)phenylmethyl)carbamide daily.

92. The method of any one of claims 81-91, wherein orally administering comprises administering 34 mg based on the free base form of N-(4-fluorophenylmethyl)-N-(1-methylpiperidin-4-yl)-N′-(4-(2-methylpropyloxy)phenylmethyl)carbamide daily.

93. The method of claim 92, wherein the 34 mg is administered in one capsule or two tablets (2×17 mg tablets).

94. The method of any one of claims 81-93, wherein the patient is concurrently being administered a CYP3A4 inhibitor, wherein orally administering comprises administering 10 mg of N-(4-fluorophenylmethyl)-N-(1-methylpiperidin-4-yl)-N′-(4-(2-methylpropyloxy)phenylmethyl)carbamide daily.

95. A method for treating selective serotonin reuptake SSRI or SNRI-induced sexual dysfunction in a human subject with SSRI or SNRI induced sexual dysfunction, the method comprising administering pimavanserin, or a pharmaceutically acceptable salt thereof, to the human subject.

96. A method for treating sexual dysfunction in a human subject with major depressive disorder comprising administering pimavanserin, or a pharmaceutically acceptable salt thereof, to the human subject.

97. The method of claim 95 or 96, wherein the pimavanserin or pharmaceutically acceptable salt thereof is administered orally to the human subject.

98. The method of any one of claims 95-97, wherein a tartrate salt of pimavanserin is administered to the human subject.

99. The method of any one of claims 95-98, wherein the pimavanserin or pharmaceutically acceptable salt thereof is administered once, twice or three times per day.

100. The method of any one of claims 95-99, wherein the pimavanserin or pharmaceutically acceptable salt thereof is administered in a daily dose between 0.5 mg to 120 mg, or between 8 mg to 42 mg.

101. The method of any one of claims 95-100, wherein the human subject is taking a SSRI or SNRI to treat MDD.

102. The method of any one of claims 95-101, wherein the human subject is on a regimen of SSRI/SNRI antidepressant as treatment for MDD.

103. The method of any one of claims 95-102, wherein the SSRI/SNRI antidepressant is citalopram, escitalopram (LEXAPRO), fluoxetine (PROZAC), fluvoxamine, paroxetine (PAXIL), sertraline, vilazodone, vortioxetine, desvenlafaxine, duloxetine, levomilnacipran, sibutramine, tramadol, milnacipran or venlafaxine.

104. The method of any one of claims 95-103, wherein the pimavanserin or pharmaceutically acceptable salt thereof is administered daily to the human subject for at least 1, 2, 3, 4, 5, 6, 7, 8, 9 or 10 or more weeks.

105. The method of any one of claims 95-104, further comprising assessing sexual dysfunction in the human subject using the Massachusetts General Hospital Sexual Functioning Index (MGH-SFI).

106. The method of any one of claims 95-105, wherein the human subject's MGH SFI score in a functional domain after the administration of the pimavanserin or pharmaceutically acceptable salt thereof is reduced as compared to the human subject's MGH-SFI score in the functional domain.

107. The method of claim 106, wherein the reduction in MGH-SFI score in the functional domain is 1, 2, 3, 4 or 5 points using a 6-point scale.

108. The method of any one of claim 106 or 107, wherein the functional domain is selected from the group consisting of “interest in sex,” “sexual arousal,” “ability to achieve orgasm,” “ability to maintain erection” [males only], and “sexual satisfaction”.

109. The method of claim 108, wherein the reduction in MGH-SFI occurs in 1, 2, 3 or 4 domains of the MGH-SFI.

110. The method of any one of claims 107-109, wherein the reduction in MGH-SFI score occurs after daily administration of the pimavanserin or pharmaceutically acceptable salt thereof for at least 2 days, 3 days, 4 days, 5 days, 6 days, 7 days, 8 days, 9 days, 10 days, 11 days, 12 days, 13 days or 14 or more days.

111. The method of any one of claims 95-110, wherein the human subject has a score of 3, 4, 5 or 6 in a 6-point scale in one, two, three or four domains of the MGH-SFI.

112. A method of reducing sexual dysfunction in a human subject on antidepressant therapy comprising administering pimavanserin, or a pharmaceutically acceptable salt thereof, to the human subject.

113. The method of claim 112, wherein the sexual dysfunction is loss of interest in sex, loss of sexual arousal, diminished ability or inability to achieve orgasm, loss of ability to maintain an erection in male subjects, loss of sexual satisfaction, reduced frequency of sexual activity, or reduced satisfaction from sexual activity.

114. The method of any one of claims 95-113, wherein after 5 weeks of daily administration, the patient has an improved score in the sexual interest item of Hamilton Depression Scale.

115. The method of any one of claims 95-113, wherein after 2 weeks of daily administration, the patient has an improved score in the sexual interest item of Hamilton Depression Scale.
